(19) United States
(12) Patent Application Publication
(10) Pub. No.: US 2023/0103789 A1
(43) Pub. Date: Apr. 6, 2023

(54) SEQUENTIAL ELECTROPORATION METHODS

(71) Applicant: MaxCyte, Inc., Gaithersburg, MD (US)

(72) Inventors: Diwash ACHARYA, Gaithersburg, MD (US); James BRADY, Gaithersburg, MD (US)

(73) Assignee: MaxCyte, Inc., Gaithersburg, MD (US)

(21) Appl. No.: 17/661,480

(22) Filed: Apr. 29, 2022

Related U.S. Application Data
(60) Provisional application No. 63/181,583, filed on Apr. 29, 2021.

(51) Int. Cl. C12N 15/87; C12M 1/42; C12N 13/00

(52) U.S. Cl. CPC C12N 15/87; C12M 35/02; C12N 13/00

(57) ABSTRACT

Aspects of the disclosure are directed to a technique for sequential electroporation that provides for a delivery of multiple electrical pulses separated in time to cells, cell particles, lipid vesicles, liposomes, or to increase efficiency of entry of one or more agents of interest into cells, cell particles, lipid vesicles, liposomes, tissues, or derivatives thereof, and to minimize damage by electrical arc or heat shock; increase loading efficiency of an agent of interest; and maintain viability of the cells, cell particles, lipid vesicles, or tissues and the ability of the cells, cell particles, lipid vesicles, liposomes, or tissues to produce a clinical effect.

### CROSS-REFERENCE TO RELATED APPLICATIONS

This application claims priority to U.S. Provisional Application Ser. No. 63/181,583, filed Apr. 29, 2021, which is incorporated by reference herein in its entirety.

### BACKGROUND

### I. Field of the Disclosure

The present disclosure relates generally to methods and apparatuses for the introduction of agents into living cells or cell particles or lipid vesicles.

### II. Background

Electroporation is the application of controlled electrical pulses for a short duration of time to transform bacteria, yeast, plant protoplasts, cultured cells, other cells, cell particles, liposomes, vesicles, tissues, or other biological vehicles. The pulse induces a transmembrane potential that causes the reversible breakdown of the cellular membrane. This action results in the permeation or “pore formation” of the cell membrane, which allows introduction of an extracellular agent, such as small molecules (such as molecular probes, drugs, dye, oligonucleotides, or peptides) or large molecules (such as proteins, DNA and RNA), into the cells, cell particles, lipid vesicles, liposomes, or tissues. This procedure is also highly efficient for the introduction of chemical or biological agents that specifically intervene in molecular pathways in tissue culture cells or primary cells, especially mammalian cells. For example, electroporation is used in the process of producing knockout mice, as well as in tumor treatment, gene therapy, and cell-based therapy.

With respect to transfection of cells, many factors contribute to the difficulty or success of transfection. For example, cellular binding and internalization of reagent-gene complexes, release of nucleic acids into the cytoplasm, the nuclear uptake and expression of a gene(s); the health, metabolic activity, rate of endocytosis, and rate of division of the cells; and the age, confluence, and passage number of cultured cells are all factors that may render cells difficult to transfect. Immature cells, including stem cells and uncommitted progenitor cells, lack these characteristics. Similarly, primary cells, which are increasingly employed as models in drug discovery, toxicology, and basic research, do not divide, have a lower internalization capacity, and often lack the ability to bind to transfection complexes.

The outcome of an electroporation process is largely controlled by the magnitude of the applied electrical field (EF) pulse and the duration of the pulse. As long as the pulse magnitude is above a certain threshold level, an increase in either the magnitude or the duration of the pulse generally results in a greater accumulation of extracellular molecules inside a cell.

Each electrical pulse applied to a cell suspension can be characterized by a certain amount of energy, which is equal to the product of voltage on the electrodes, current through the buffer, and duration of the high voltage pulse. However, only a small percentage of applied electrical energy is spent on the useful work of modifying lipid membranes and moving extracellular materials into cells. The rest of electrical energy dissipates in the form of heat that is produced in the surrounding media. Power dissipation that slightly heats the cell suspension is an inevitable consequence of applying EF, even though heating itself does not cause permeabilization of cells. The more conductive the electroporation buffer is, the more energy is wasted on heat production. Buildup of heat to temperatures greater than 20-24 degrees above ambient temperature can cause permanent damage to cells and cell components and decrease the efficiency of the electroporation process; this limits the amount of energy capable of being used for successful electroporation of cells, cell particles, lipid vesicles, liposomes, or tissues.

Temperature increases in electroporation samples are also related to an increase in the electrical conductivity of the samples, which in simple salt solutions increases by about 2% per ° C. Applied electrical field causes a current flow through the cell or particle suspension, which causes a temperature rise that translates into a conductivity increase and a greater current draw from the power source, and so on. If such positive feedback process is not interrupted (e.g., by switching the pulse off), the current increase proceeds in an avalanche-like manner and results in arcing and sample loss. This effect is mainly observed at relatively high field strengths (>2 kV/cm).

Electroporation of difficult-to-transfect cells, such as immature or primary cells, for example, requires strong electrical fields, and therefore either buffer conductivity or pulse width must be limited. However, cells are extremely sensitive to environmental biochemical changes, and the physico-chemical changes in the environment associated with application of electrical field to cells, cell particles, lipid vesicles, or tissues may modulate the physiological state, activation properties, and biological function of the cells, cell particles, lipid vesicles, liposomes, or tissues, impacting the ability of the electroporated materials to deliver clinical effect.

Therefore, the inventors believe there is a need for methods to electroporate difficult-to-transfect cells, cell particles, lipid vesicles, or tissues with agents of interest at a high efficiency without damaging the cells, cell particles, lipid vesicles, liposomes, or tissues beyond their ability to produce a clinical effect.

### SUMMARY

Described herein, in some aspects, are methods and apparatuses for the efficient electroporation of difficult-to-transfect cells, cell particles, lipid vesicles, liposomes, or tissues with agents of interest using a novel electroporation protocol comprising sequential electrical pulses. In certain aspects, sequential electroporation of cells, cell particles, lipid vesicles, liposomes, or tissues surprisingly leads to significantly higher transgene expression versus single electroporation of cells, cell particles, lipid vesicles, liposomes, or tissues. In certain aspects, the methods and apparatuses described herein are unique because they can increase the loading efficiency of an agent of interest into cells, cell particles, lipid vesicles, liposomes, or tissues while maintaining viability of the cells, cell particles, lipid vesicles, or tissues and maintaining the ability of the cells, cell particles, lipid vesicles, liposomes, or tissues to produce a clinical effect. In certain aspects, the methods and apparatuses disclosed herein can optimize efficiency and viability following sequential electroporation by varying the electroporation energy used during each round of electroporation.

Aspects of the present disclosure relate to methods of transfecting agents of interest; methods of transiently permeabilizing membranes to allow transport of agents of interest through the membranes; methods of electroporating cells, cell particles, lipid vesicles, liposomes, or tissues; methods of producing electroporated cells, cell particles, lipid vesicles, liposomes, or tissues; and methods of increasing efficiency of electroporation while maintaining clinical effect of electroporated materials. The steps and aspects discussed in this disclosure are contemplated as part of any of these methods. In some aspects, the methods contemplated herein can comprise or exclude 1, 2, 3, 4, 5, or more of the following steps: subjecting a sample comprising one or more intact cells, cell particles, or lipid vesicles to a first electrical pulse having a first field strength and a first pulse duration sufficient to load the cells, cell particles, or lipid vesicles with an agent according to a first protocol; subjecting the sample to a second electrical pulse having a second field strength and a second pulse duration sufficient to load the cells, cell particles, or lipid vesicles with an agent according to a second protocol; subjecting the sample to a time delay between a first electrical pulse and a second electrical pulse; providing in the sample a nucleic acid, polypeptide, protein, or small molecule; subjecting cells, cell particles, or lipid vesicles to conditions sufficient to electroporate the cells, cell particles, or lipid vesicles; expressing an electroporated nucleic acid, polypeptide, protein in a cell; and modifying the loading efficiency of an agent(s) into a sample(s), the clinical effect of an electroporated sample(s), and/or sample viability by modifying the electrical parameters of and/or time delay between electrical pulses to which the samples are subjected. Any one or more of these steps may be excluded from the disclosed methods.

Aspects of the present disclosure include electroporation methods comprising:

(1) subjecting a sample comprising one or more intact cells, cell particles, or lipid vesicles to a first electrical pulse having a first field strength and a first pulse duration sufficient to load the cells, cell particles, or lipid vesicles with a first agent according to a first protocol; and subjecting the sample to a second electrical pulse having a second field strength and a second pulse duration sufficient to load the cells, cell particles, or lipid vesicles with a second agent according to a second protocol; wherein the first field strength and/or the first pulse duration are different from the second field strength and/or second pulse duration;

(2) subjecting a sample comprising one or more intact cells, cell particles, or lipid vesicles to a first electrical pulse having a first field strength and a first pulse duration sufficient to load the cells, cell particles, or lipid vesicles with a first agent according to a first protocol; allowing the sample to recover for at least about 24 hours; and subjecting the sample to a second electrical pulse having a second field strength and a second pulse duration sufficient to load the cells, cell particles, or lipid vesicles with a second agent according to a second protocol;

(3) subjecting a sample comprising one or more intact cells, cell particles, or lipid vesicles to a first electrical pulse having a first field strength and a first pulse duration sufficient to load the cells, cell particles, or lipid vesicles with a first agent according to a first protocol; allowing the sample to recover for at least about 24 hours; and subjecting the sample to a second electrical pulse having a second field strength and a second pulse duration sufficient to load the cells, cell particles, or lipid vesicles with a second agent according to a second protocol; wherein the first field strength and/or the first pulse duration are different from the second field strength and/or second pulse duration;

(4) subjecting a sample comprising one or more intact cells, cell particles, or lipid vesicles to a first electrical pulse having a first field strength and a first pulse duration sufficient to load the cells, cell particles, or lipid vesicles with a first agent; and subjecting the sample to a second electrical pulse having a second field strength and a second pulse duration sufficient to load the cells, cell particles, or lipid vesicles with a second agent; wherein the first field strength and/or the first pulse duration are different from the second field strength and/or second pulse duration;

(5) subjecting a sample comprising one or more intact cells, cell particles, or lipid vesicles to a first electrical pulse having a first field strength and a first pulse duration sufficient to load the cells, cell particles, or lipid vesicles with a first agent; allowing the sample to recover for at least about 24 hours; and subjecting the sample to a second electrical pulse having a second field strength and a second pulse duration sufficient to load the cells, cell particles, or lipid vesicles with a second agent; or

(6) subjecting a sample comprising one or more intact cells, cell particles, or lipid vesicles to a first electrical pulse having a first field strength and a first pulse duration sufficient to load the cells, cell particles, or lipid vesicles with a first agent; allowing the sample to recover for at least about 24 hours; and subjecting the sample to a second electrical pulse having a second field strength and a second pulse duration sufficient to load the cells, cell particles, or lipid vesicles with a second agent; wherein the first field strength and/or the first pulse duration are different from the second field strength and/or second pulse duration.

Aspects of the present disclosure include methods of serially editing cell genes comprising:

(1) subjecting a sample comprising one or more intact cells to a first electrical pulse having a first field strength and a first pulse duration sufficient to load the cells with a first agent according to a first protocol; and subjecting the sample to a second electrical pulse having a second field strength and a second pulse duration sufficient to load the cells with a second agent according to a second protocol; wherein the first field strength and/or the first pulse duration are different from the second field strength and/or second pulse duration;

(2) subjecting a sample comprising one or more intact cells to a first electrical pulse having a first field strength and a first pulse duration sufficient to load the cells with a first agent according to a first protocol; allowing the sample to recover for at least about 24 hours; and subjecting the sample to a second electrical pulse having a second field strength and a second pulse duration sufficient to load the cells with a second agent according to a second protocol;

(3) subjecting a sample comprising one or more intact cells to a first electrical pulse having a first field strength and a first pulse duration sufficient to load the cells with a first agent according to a first protocol; allowing the sample to recover for at least about 24 hours; and subjecting the sample to a second electrical pulse having a second field strength and a second pulse duration sufficient to load the cells with a second agent according to a second protocol; wherein the first field strength and/or the first pulse duration are different from the second field strength and/or second pulse duration;

(4) subjecting a sample comprising one or more intact cells to a first electrical pulse having a first field strength and a first pulse duration sufficient to load the cells with a first agent; and subjecting the sample to a second electrical pulse having a second field strength and a second pulse duration sufficient to load the cells with a second agent; wherein the first field strength and/or the first pulse duration are different from the second field strength and/or second pulse duration;

(5) subjecting a sample comprising one or more intact cells to a first electrical pulse having a first field strength and a first pulse duration sufficient to load the cells with a first agent; allowing the sample to recover for at least about 24 hours; and subjecting the sample to a second electrical pulse having a second field strength and a second pulse duration sufficient to load the cells with a second agent; or

(6) subjecting a sample comprising one or more intact cells to a first electrical pulse having a first field strength and a first pulse duration sufficient to load the cells with a first agent; allowing the sample to recover for at least about 24 hours; and subjecting the sample to a second electrical pulse having a second field strength and a second pulse duration sufficient to load the cells with a second agent; wherein the first field strength and/or the first pulse duration are different from the second field strength and/or second pulse duration.

Further aspects of the present disclosure include electroporation methods comprising:

(1) subjecting a cell sample comprising one or more intact cells to a first electrical pulse having a first field strength and a first pulse duration sufficient to load cells with a first agent comprising RNA according to a first protocol; allowing the cell sample to recover for at least about 24 hours; and subjecting the cell sample to a second electrical pulse having a second field strength and a second pulse duration sufficient to load cells with a second agent comprising RNA according to a second protocol; wherein the first field strength and/or the first pulse duration are different from the second field strength and/or second pulse duration;

(2) subjecting a cell sample comprising one or more intact cells to a first electrical pulse having a first field strength and a first pulse duration sufficient to load cells with a first agent comprising DNA according to a first protocol; allowing the cell sample to recover for at least about 24 hours; and subjecting the cell sample to a second electrical pulse having a second field strength and a second pulse duration sufficient to load cells with a second agent comprising DNA according to a second protocol; wherein the first field strength and/or the first pulse duration are different from the second field strength and/or second pulse duration; or

(3) subjecting a cell sample comprising one or more intact cells to a first electrical pulse having a first field strength and a first pulse duration sufficient to load cells with a first agent comprising one or more proteins according to a first protocol; allowing the cell sample to recover for at least about 24 hours; and subjecting the cell sample to a second electrical pulse having a second field strength and a second pulse duration sufficient to load cells with a second agent comprising one or more proteins according to a second protocol; wherein the first field strength and/or the first pulse duration are different from the second field strength and/or second pulse duration; or

(4) subjecting a cell sample comprising one or more intact cells to a first electrical pulse having a first field strength and a first pulse duration sufficient to load cells with a first agent comprising a ribonucleoprotein according to a first protocol; allowing the cell sample to recover for at least about 24 hours; and subjecting the cell sample to a second electrical pulse having a second field strength and a second pulse duration sufficient to load cells with a second agent comprising a ribonucleoprotein according to a second protocol; wherein the first field strength and/or the first pulse duration are different from the second field strength and/or second pulse duration; or

(5) subjecting a cell sample comprising one or more intact cells to a first electrical pulse having a first field strength and a first pulse duration sufficient to load cells with a first agent comprising RNA; allowing the cell sample to recover for at least about 24 hours; and subjecting the cell sample to a second electrical pulse having a second field strength and a second pulse duration sufficient to load cells with a second agent comprising RNA; wherein the first field strength and/or the first pulse duration are different from the second field strength and/or second pulse duration;

(6) subjecting a cell sample comprising one or more intact cells to a first electrical pulse having a first field strength and a first pulse duration sufficient to load cells with a first agent comprising DNA; allowing the cell sample to recover for at least about 24 hours; and subjecting the cell sample to a second electrical pulse having a second field strength and a second pulse duration sufficient to load cells with a second agent comprising DNA; wherein the first field strength and/or the first pulse duration are different from the second field strength and/or second pulse duration; or

(7) subjecting a cell sample comprising one or more intact cells to a first electrical pulse having a first field strength and a first pulse duration sufficient to load cells with a first agent comprising one or more proteins; allowing the cell sample to recover for at least about 24 hours; and subjecting the cell sample to a second electrical pulse having a second field strength and a second pulse duration sufficient to load cells with a second agent comprising one or more proteins; wherein the first field strength and/or the first pulse duration are different from the second field strength and/or second pulse duration; or

(8) subjecting a cell sample comprising one or more intact cells to a first electrical pulse having a first field strength and a first pulse duration sufficient to load cells with a first agent comprising a ribonucleoprotein; allowing the cell sample to recover for at least about 24 hours; and subjecting the cell sample to a second electrical pulse having a second field strength and a second pulse duration sufficient to load cells with a second agent comprising a ribonucleoprotein; wherein the first field strength and/or the first pulse duration are different from the second field strength and/or second pulse duration.

In some aspects, the first and second agent are the same agent. In some aspects, the first and second agent are different agents. In some aspects, the first and second agents are a nucleic acid, polypeptide, protein, or small molecule. In some aspects, the nucleic acid is RNA, and wherein the RNA is mRNA, miRNA, shRNA, siRNA, or an antisense oligonucleotide. In some aspects, the nucleic acid is DNA, and wherein the DNA is an antisense oligonucleotide, a vector, or a double sense linear DNA. In some aspects, the protein is a ribonucleoprotein. In some aspects, the ribonucleoprotein comprises a Cas9 protein and a guide RNA.

In some aspects, the method further comprises a resting step after the first and/or second electrical pulses. In some aspects, the resting step comprises incubation of the sample for 10-30 minutes. In some aspects, the resting step comprises incubation of the sample at 25-50° C. In some aspects, the resting step comprises incubation of the sample at 3-8% CO₂. In some aspects, the sample is not subjected to a resting step after the first and/or second electrical pulses.

In some aspects, the first field strength equals the second field strength, and the first pulse duration is longer than the second pulse duration. In some aspects, the first field strength equals the second field strength, and the first pulse duration is shorter than the second pulse duration. In some aspects, the first field strength equals the second field strength, and the first pulse duration equals the second pulse duration. In some aspects, the first field strength is less than the second field strength, and the first pulse duration equals the second pulse duration. In some aspects, the first field strength is greater than the second field strength, and the first pulse duration equals the second pulse duration. In some aspects, the first field strength is less than the second field strength, and the first pulse duration is longer than the second pulse duration. In some aspects, the first field strength is greater than the second field strength, and the first pulse duration longer than the second pulse duration. In some aspects, the first field strength is less than the second field strength, and the first pulse duration is shorter than the second pulse duration. In some aspects, the first field strength is greater than the second field strength, and the first pulse duration is shorter than the second pulse duration.

In some aspects, the field strength of the first electrical pulse and pulse duration of the first electrical pulse produce a first total applied electrical energy, and the field strength of the second electrical pulse and pulse duration of the second electrical pulse produce a second total applied electrical energy, and the first total applied electrical energy is greater than the second total applied electrical energy. In some aspects, the first total applied electrical energy is less than the second total applied electrical energy.

In some aspects, the first and second field strengths of the first and second electrical pulses are a function of a voltage magnitude of the electrical pulses, duration of the electrical pulses, and a conductivity of the sample. The voltage magnitude of the electrical pulses can be between 0.001 Volts and 10,000 Volts, 0.01 Volts and 10,000 Volts, 0.1 Volts and 10,000 Volts, 1 Volt and 10,000 Volts, 1 Volt and 9,000 Volts, 1 Volt and 8,000 Volts, 1 Volt and 7,000 Volts, 1 Volt and 6,000 Volts, 1 Volt and 5,000 Volts, 1 Volt and 4,000 Volts, 1 Volt and 3,000 Volts, 1 Volt and 2,000 Volts, or 1 Volt and 1,000 Volts. In some aspects, the voltage magnitude of the electrical pulses is between 100 Volts and 900 Volts. In some aspects, the conductivity of the sample is a function of parameters comprising ionic composition of electroporation buffer, concentration of an agent to be loaded into the cells, cell density, temperature, and pressure. The conductivity of the sample can be between 0.01 Siemens/meter and 10 Siemens/meter, 0.01 Siemens/meter and 1 Siemens/meter, 0.1 Siemens/meter and 10 Siemens/meter, 0.1 Siemens/meter and 1 Siemens/meter, or 1 Siemens/meter and 10 Siemens/meter. In some aspects, the conductivity of the sample is between 1.0 and 3.0 Siemens/meter. In some aspects, the first and second field strengths are further a function of a geometry of an electroporation chamber. The electroporation chamber can comprise an electrode gap between 0.001 cm and 10 cm, 0.001 cm and 1 cm, 0.01 cm and 10 cm, 0.01 cm and 1 cm, 0.1 cm and 10 cm, 0.1 cm and 1 cm, or 1 cm and 10 cm. In some aspects, the electroporation chamber comprises an electrode gap between 0.01 cm and 1 cm.

The first and second field strengths of the first and second electrical pulses can be between 0.001 kV/cm and 10 kV/cm, 0.01 kV/cm and 1 kV/cm, 0.1 kV/cm and 10 kV/cm, 0.1 kV/cm and 1 kV/cm, or 1 kV/cm and 10 kV/cm. In some aspects, the first and second field strengths of the first and second electrical pulses are between 0.3 kV/cm and 3 kV/cm.

The first and second pulse durations of the first and second electrical pulses can be between 10⁻⁶ seconds and 10 seconds, 10⁻⁶ seconds and 1 second, 10⁻³ seconds and 10 seconds, or 10⁻³ seconds and 1 second. In some aspects, the first and second pulse durations of the first and second electrical pulses are between 1 microsecond and 100 milliseconds.

In some aspects, the first and second electrical pulses further comprise characteristics relating to pulse number, width, shape, pattern, or polarity. The pulse number can be between 1 pulse and 1000 pulses, 1 pulse and 900 pulses, 1 pulse and 800 pulses, 1 pulse and 700 pulses, 1 pulse and 600 pulses, 1 pulse and 500 pulses, 1 pulse and 400 pulses, 1 pulse and 300 pulses, 1 pulse and 200 pulses, 1 pulse and 100 pulses, 1 pulse and 90 pulses, 1 pulse and 80 pulses, 1 pulse and 70 pulses, 1 pulse and 60 pulses, 1 pulse and 50 pulses, 1 pulse and 40 pulses, 1 pulse and 30 pulses, 1 pulse and 20 pulses, or 1 pulse and 10 pulses. In some aspects, the pulse number is between 1 pulse and 130 pulses.

In some aspects, the pulse width is a function of a rate of exponential decay. In some aspects, the rate of exponential decay is a function of a resistance of the sample and a capacitance of a power supply used to effect electroporation. The resistance of the sample can be between 1 ohm and 10000 ohms, 1 ohm and 9000 ohms, 1 ohm and 8000 ohms, 1 ohm and 7000 ohms, 1 ohm and 6000 ohms, 1 ohm and 5000 ohms, 1 ohm and 4000 ohms, 1 ohm and 3000 ohms, 1 ohm and 2000 ohms, 1 ohm and 1000 ohms, 1 ohm and 900 ohms, 1 ohm and 800 ohms, 1 ohm and 700 ohms, 1 ohm and 600 ohms, 1 ohm and 500 ohms, 1 ohm and 400 ohms, 1 ohm and 300 ohms, 1 ohm and 200 ohms, 1 ohm and 100 ohms, 1 ohm and 90 ohms, 1 ohm and 80 ohms, 1 ohm and 70 ohms, 1 ohm and 60 ohms, 1 ohm and 50 ohms, 1 ohm and 40 ohms, 1 ohm and 30 ohms, 1 ohm and 20 ohms, or 1 ohm and 10 ohms. In some aspects, the resistance of the sample is between 1 ohm and 1000 ohms. The power supply capacitance can be between 1 μF and 1,000,000 μF, 1 μF and 100,000 μF, 1μF and 10,000 μF, 1 μF and 1,000 μF, or 1 μF and 100 μF. In some aspects, the power supply capacitance is between 1000 μF and 5000 μF.

In some aspects, the pulse shape is a square wave pulse or an exponential decay wave pulse. In some aspects, the pulse pattern comprises a single pulse corresponding to the duration of the first or second pulse. In some aspects, the pulse pattern comprises multiple pulses, wherein a combined duration of the multiple pulses corresponds to the duration of the first or second pulse. In some aspects, the polarity of the first and second electrical pulses is positive or negative.

In some aspects, the sample is subjected to the second electrical pulse at least about 12 hours to at least about 48 hours after the sample is subjected to the first pulse. In some aspects, the sample is subjected to the second electrical pulse at least about 24 hours after the sample is subjected to the first pulse.

The methods can be performed by an electroporation system having a non-transitory computer readable medium comprising instructions that, when executed by a processor, cause the processor to execute the first and second protocols to electroporate the sample. In some aspects, the electroporation system comprises a flow electroporation apparatus and the sample is subjected to the electrical pulses while the sample is flowing within the flow electroporation apparatus.

In some aspects, the cells can be mammalian cells, and in some aspects, the cells are human cells, murine cells, rat cells, hamster cells, or primate cells. In some aspects, the cells are primary cells. In some aspects, the cells are cultured cells, and the cultured cells can be cultured cell lines which can comprise 3T3, 697, 10T½, 1321N1, A549, AHR77, B-LCL, B16, B65, Ba/F3, BHK, C2C12, C6, CaCo-2, CAP, CaSki, ChaGo-K-1, CHO, COS, DG75, DLD-1, EL4, H1299, HaCaT, HAP1, HCT116, HEK, HeLa, HepG2, HL60, HOS, HT1080, HT29, Huh-7, HUVEC, INS-1/GRINCH, Jurkat, K46, K562, KG1, KHYG-1, L5278Y, L6, LNCaP, LS180, MCF7, MDA-MB-231, ME-180, MG-63, Min-6, MOLT4, Nalm6, ND7/23, Neuro2a, NK92, NS/0, P3U1, Panc-1, PC-3, PC12, PER.C6, PM1, Ramos, RAW 264.7, RBL, Renca, RLE, SH-SY5Y, SK-BR-3, SK-MES-1, SK-N-SH, SK-OV-3, SP2/0, SW403, THP-1, U2OS, U937, Vero, YB2/0, or derivatives thereof. The cells can comprise adipocytes, chondrocytes, endothelial cells, epithelial cells, fibroblasts, hepatocytes, keratinocytes, myocytes, neurons, osteocytes, peripheral blood mononuclear cells (PBMCs), splenocytes, stem cells, or thymocytes. In some aspects, the PBMCs are peripheral blood lymphocytes (PBLs), which can be natural killer (NK) cells, T cells, or B cells. In some aspects, the PBMCs are monocytes, which can be macrophages or dendritic cells, and the macrophages can be microglia. In some aspects, the stem cells are adipose stem cells, embryonic stem cells, hematopoietic stem cells, induced pluripotent stem cells, mesenchymal stem cells, or neural stem cells.

In some aspects, a loading efficiency of the agent is at least 50, 60, 70, 80, or 90%.

In some aspects, cell viability can be at least 50% 12 to 96 hours after the second electrical pulse; at least 60% 12 to 96 hours after the second electrical pulse; at least 70% 12 to 96 hours after the second electrical pulse; at least 80% 12 to 96 hours after the second electrical pulse; or at least 90% 12 to 96 hours after the second electrical pulse.

In some aspects, the electroporated cells are approximately 50% to 90% viable 12 to 96 hours after the second electrical pulse; approximately 50% to 90% viable 12 to 72 hours after the second electrical pulse; approximately 50% to 90% viable 12 to 48 hours after the second electrical pulse; approximately 50% to 90% viable 24 hours after the second electrical pulse; approximately 60% to 90% viable 12 to 96 hours after the second electrical pulse; approximately 60% to 90% viable 12 to 72 hours after the second electrical pulse; approximately 60% to 90% viable 12 to 48 hours after the second electrical pulse; or approximately 60% to 90% viable 24 hours after the second electrical pulse.

Aspects of the disclosure also relate to an electroporation system having a non-transitory computer readable medium comprising instructions that, when executed by a processor, cause the processor to: select a first protocol associated with a first electrical pulse having a first field strength and a first pulse duration; subject a sample comprising one or more intact cells, cell particles, or lipid vesicles to the first electrical pulse defined by the first protocol sufficient to load the cells, cell particles, or lipid vesicles with a first agent according to the first protocol; select a second protocol associated with a second electrical pulse having a second field strength and a second pulse duration; and subject the sample to the second electrical pulse defined by the second protocol sufficient to load the cells, cell particles, or lipid vesicles with a second agent according to the second protocol; wherein the first field strength and/or the first pulse duration are different from the second field strength and/or second pulse duration.

Aspects of the disclosure also include an electroporated cell, cell particle, or lipid vesicle produced using any method described herein or produced using any electroporation system having a non-transitory computer readable medium comprising instructions that, when executed by a processor, cause the processor to execute any method described herein.

Further aspects of the disclosure include a method of treating a subject having or suspected of having a disease or condition comprising administering a product of any of the methods described in an amount that mitigates the disease or condition. In certain aspects, a product of the methods is a drug delivery vehicle, and it is contemplated that a wide variety of known drugs can be delivered via loaded particles produced by the methods described. A disease or condition can include any disease or condition amenable to the delivery of a drug or agent via liposome particle, cell particle, or similar delivery vehicle that is prepared (e.g., loaded) by using electroporation methods.

Still further aspects of the disclosure include methods of treating a subject having or suspected of having a disease or condition by administering an effective amount of a drug, a biologic or other bioactive molecule comprised in a particle produced by the methods described. In certain aspects, the disease is an infectious disease, including but not limited to a bacterial, fungal, parasitic, or viral infection. In a further aspect the bacterial infection is a mycobacterial infection. In still a further aspect, the viral infection is a retroviral infection including but not limited to HIV infection. In another aspect, the disease is an inflammatory disease or cancer or vascular occlusive disease.

Aspects of the disclosure include an electroporation system configured to perform any of the methods described.

Also disclosed are the following aspects 1 to 201 of the present disclosure.

Aspect 1 is an electroporation method comprising: subjecting a sample comprising one or more intact cells, cell particles, or lipid vesicles to a first electrical pulse having a first field strength and a first pulse duration sufficient to load the cells, cell particles, or lipid vesicles with a first agent according to a first protocol; and subjecting the sample to a second electrical pulse having a second field strength and a second pulse duration sufficient to load the cells, cell particles, or lipid vesicles with a second agent according to a second protocol; wherein the first field strength and/or the first pulse duration are different from the second field strength and/or second pulse duration.

Aspect 2 is an electroporation method comprising: subjecting a sample comprising one or more intact cells, cell particles, or lipid vesicles to a first electrical pulse having a first field strength and a first pulse duration sufficient to load the cells, cell particles, or lipid vesicles with a first agent according to a first protocol; allowing the sample to recover for at least about 24 hours; and subjecting the sample to a second electrical pulse having a second field strength and a second pulse duration sufficient to load the cells, cell particles, or lipid vesicles with a second agent according to a second protocol.

Aspect 3 is an electroporation method comprising: subjecting a sample comprising one or more intact cells, cell particles, or lipid vesicles to a first electrical pulse having a first field strength and a first pulse duration sufficient to load the cells, cell particles, or lipid vesicles with a first agent according to a first protocol; allowing the sample to recover for at least about 24 hours; and subjecting the sample to a second electrical pulse having a second field strength and a second pulse duration sufficient to load the cells, cell particles, or lipid vesicles with a second agent according to a second protocol; wherein the first field strength and/or the first pulse duration are different from the second field strength and/or second pulse duration.

Aspect 4 is a method of serially editing cell genes comprising: subjecting a sample comprising one or more intact cells to a first electrical pulse having a first field strength and a first pulse duration sufficient to load the cells with a first agent according to a first protocol; and subjecting the sample to a second electrical pulse having a second field strength and a second pulse duration sufficient to load the cells with a second agent according to a second protocol; wherein the first field strength and/or the first pulse duration are different from the second field strength and/or second pulse duration.

Aspect 5 is a method of serially editing cell genes comprising: subjecting a sample comprising one or more intact cells to a first electrical pulse having a first field strength and a first pulse duration sufficient to load the cells with a first agent according to a first protocol; allowing the sample to recover for at least about 24 hours; and subjecting the sample to a second electrical pulse having a second field strength and a second pulse duration sufficient to load the cells with a second agent according to a second protocol.

Aspect 6 is a method of serially editing cell genes comprising: subjecting a sample comprising one or more intact cells to a first electrical pulse having a first field strength and a first pulse duration sufficient to load the cells with a first agent according to a first protocol; allowing the sample to recover for at least about 24 hours; and subjecting the sample to a second electrical pulse having a second field strength and a second pulse duration sufficient to load the cells with a second agent according to a second protocol; wherein the first field strength and/or the first pulse duration are different from the second field strength and/or second pulse duration.

Aspect 7 is the method of Aspects 1 to 6, wherein the first and second agent are the same agent. Aspect 8 is the method of Aspects to 1 to 7, wherein the first and second agent are different agents. Aspect 9 is the method of Aspects 1 to 8, wherein the first and second agents are a nucleic acid, polypeptide, protein, or small molecule. Aspect 10 is the method of Aspects 1 to 9, wherein the first agent is a nucleic acid, polypeptide, protein, or small molecule, and wherein the second agent is a nucleic acid, polypeptide, protein, or small molecule. Aspect 11 is the method of Aspects 1 to 10, wherein the first agent is a nucleic acid, and wherein the second agent is a nucleic acid, polypeptide, protein, or small molecule. Aspect 12 is the method of Aspects 1 to 10, wherein the first agent is a polypeptide, and wherein the second agent is a nucleic acid, polypeptide, protein, or small molecule. Aspect 13 is the method of Aspects 1 to 10, wherein the first agent is a protein, and wherein the second agent is a nucleic acid, polypeptide, protein, or small molecule. Aspect 14 is the method of Aspects 1 to 10, wherein the first agent is a small molecule, and wherein the second agent is a nucleic acid, polypeptide, protein, or small molecule. Aspect 15 is the method of Aspects 1 to 14, wherein the first agent is a nucleic acid, polypeptide, protein, or small molecule, and wherein the second agent is a nucleic acid. Aspect 16 is the method of Aspects 1 to 14, wherein the first agent is a nucleic acid, polypeptide, protein, or small molecule, and wherein the second agent is a polypeptide. Aspect 17 is the method of Aspects 1 to 14, wherein the first agent is a nucleic acid, polypeptide, protein, or small molecule, and wherein the second agent is a protein. Aspect 18 is the method of Aspects 1 to 14, wherein the first agent is a nucleic acid, polypeptide, protein, or small molecule, and wherein the second agent is a small molecule. Aspect 19 is the method of Aspects 1 to 18, wherein the nucleic acid is RNA, and wherein the RNA is mRNA, miRNA, shRNA, siRNA, or an antisense oligonucleotide. Aspect 20 is the method of Aspects 1 to 19, wherein the nucleic acid is DNA, and wherein the DNA is an antisense oligonucleotide, a vector, or a double sense linear DNA. Aspect 21 is the method of Aspects 1 to 20, wherein the protein is a ribonucleoprotein. Aspect 22 is the method of Aspects 1 to 21, wherein the ribonucleoprotein comprises a Cas9 protein and a guide RNA.

Aspect 23 is an electroporation method comprising: (a) subjecting a cell sample comprising one or more intact cells to a first electrical pulse having a first field strength and a first pulse duration sufficient to load cells with a first agent comprising RNA according to a first protocol; (b) allowing the cell sample to recover for at least about 24 hours; and (c) subjecting the cell sample to a second electrical pulse having a second field strength and a second pulse duration sufficient to load cells with a second agent comprising RNA according to a second protocol; wherein the first field strength and/or the first pulse duration are different from the second field strength and/or second pulse duration.

Aspect 24 is an electroporation method comprising: (a) subjecting a cell sample comprising one or more intact cells to a first electrical pulse having a first field strength and a first pulse duration sufficient to load cells with a first agent comprising DNA according to a first protocol; (b) allowing the cell sample to recover for at least about 24 hours; and (c) subjecting the cell sample to a second electrical pulse having a second field strength and a second pulse duration sufficient to load cells with a second agent comprising DNA according to a second protocol; wherein the first field strength and/or the first pulse duration are different from the second field strength and/or second pulse duration.

Aspect 25 is an electroporation method comprising: (a) subjecting a cell sample comprising one or more intact cells to a first electrical pulse having a first field strength and a first pulse duration sufficient to load cells with a first agent comprising one or more proteins according to a first protocol; (b) allowing the cell sample to recover for at least about 24 hours; and (c) subjecting the cell sample to a second electrical pulse having a second field strength and a second pulse duration sufficient to load cells with a second agent comprising one or more proteins according to a second protocol; wherein the first field strength and/or the first pulse duration are different from the second field strength and/or second pulse duration.

Aspect 26 is a method of serially editing cells comprising: (a) subjecting a cell sample comprising one or more intact cells to a first electrical pulse having a first field strength and a first pulse duration sufficient to load cells with a first agent comprising a ribonucleoprotein according to a first protocol; (b) allowing the cell sample to recover for at least about 24 hours; and (c) subjecting the cell sample to a second electrical pulse having a second field strength and a second pulse duration sufficient to load cells with a second agent comprising a ribonucleoprotein according to a second protocol; wherein the first field strength and/or the first pulse duration are different from the second field strength and/or second pulse duration.

Aspect 27 is the method of Aspects 23 to 26, wherein the first and second agent are the same agent. Aspect 28 is the method of Aspects 23 to 26, wherein the first and second agent are different agents. Aspect 29 is the method of Aspects 23 to 28, wherein the first and second agents are a nucleic acid, polypeptide, protein, or small molecule. Aspect 30 is the method of Aspects 23 to 29, wherein the first agent is a nucleic acid, polypeptide, protein, or small molecule, and wherein the second agent is a nucleic acid, polypeptide, protein, or small molecule. Aspect 31 is the method of Aspects 23 to 30, wherein the first agent is a nucleic acid, and wherein the second agent is a nucleic acid, polypeptide, protein, or small molecule. Aspect 32 is the method of Aspects 23 to 30, wherein the first agent is a polypeptide, and wherein the second agent is a nucleic acid, polypeptide, protein, or small molecule. Aspect 33 is the method of Aspects 23 to 30, wherein the first agent is a protein, and wherein the second agent is a nucleic acid, polypeptide, protein, or small molecule. Aspect 34 is the method of Aspects 23 to 30, wherein the first agent is a small molecule, and wherein the second agent is a nucleic acid, polypeptide, protein, or small molecule. Aspect 35 is the method of Aspects 23 to 34, wherein the first agent is a nucleic acid, polypeptide, protein, or small molecule, and wherein the second agent is a nucleic acid. Aspect 36 is the method of Aspects 23 to 34, wherein the first agent is a nucleic acid, polypeptide, protein, or small molecule, and wherein the second agent is a polypeptide. Aspect 37 is the method of Aspects 23 to 34, wherein the first agent is a nucleic acid, polypeptide, protein, or small molecule, and wherein the second agent is a protein. Aspect 38 is the method of Aspects 23 to 34, wherein the first agent is a nucleic acid, polypeptide, protein, or small molecule, and wherein the second agent is a small molecule. Aspect 39 is the method of Aspects 23 to 38, wherein the nucleic acid is RNA, and wherein the RNA is mRNA, miRNA, shRNA, siRNA, or an antisense oligonucleotide. Aspect 40 is the method of Aspects 23 to 39, wherein the nucleic acid is DNA, and wherein the DNA is an antisense oligonucleotide, a vector, or a double sense linear DNA. Aspect 41 is the method of Aspects 23 to 40, wherein the protein is a ribonucleoprotein. Aspect 42 is the method of Aspects 23 to 41, wherein the ribonucleoprotein comprises a Cas9 protein and a guide RNA.

Aspect 43 is the method of Aspects 1 to 42, further comprising a resting step after the first and/or second electrical pulses. Aspect 44 is the method of Aspects 1 to 43, further comprising a resting step after the first and/or second electrical pulses, wherein the resting step comprises incubation of the sample for 10-30 minutes. Aspect 45 is the method of Aspects 1 to 44, further comprising a resting step after the first and/or second electrical pulses, wherein the resting step comprises incubation of the sample at 25-50° C. Aspect 46 is the method of Aspects 1 to 45, further comprising a resting step after the first and/or second electrical pulses, wherein the resting step comprises incubation of the sample at 3-8% CO₂. Aspect 47 is the method of Aspects 1 to 46, wherein the sample is not subjected to a resting step after the first and/or second electrical pulses. Aspect 48 is the method of Aspects 1 to 47, wherein the first field strength equals the second field strength, and wherein the first pulse duration is longer than the second pulse duration. Aspect 49 is the method of Aspects 1 to 47, wherein the first field strength equals the second field strength, and wherein the first pulse duration is shorter than the second pulse duration. Aspect 50 is the method of Aspects 1 to 47, wherein the first field strength is less than the second field strength, and wherein the first pulse duration equals the second pulse duration. Aspect 51 is the method of Aspects 1 to 47, wherein the first field strength is greater than the second field strength, and wherein the first pulse duration equals the second pulse duration. Aspect 52 is the method of Aspects 1 to 47, wherein the first field strength is less than the second field strength, and wherein the first pulse duration is longer than the second pulse duration. Aspect 53 is the method of Aspects 1 to 47, wherein the first field strength is greater than the second field strength, and wherein the first pulse duration longer than the second pulse duration. Aspect 54 is the method of Aspects 1 to 47, wherein the first field strength is less than the second field strength, and wherein the first pulse duration is shorter than the second pulse duration. Aspect 55 is the method of Aspects 1 to 47, wherein the first field strength is greater than the second field strength, and wherein the first pulse duration is shorter than the second pulse duration. Aspect 56 is the method of Aspects 1 to 55, wherein the first field strength and first pulse duration produce a first total applied electrical energy and the second field strength and second pulse duration produce a second total applied electrical energy, and wherein the first total applied electrical energy is different than the second total applied electrical energy. Aspect 57 is the method of Aspects 1 to 56, wherein the first field strength and first pulse duration produce a first total applied electrical energy and the second field strength and second pulse duration produce a second total applied electrical energy, wherein the first total applied electrical energy is different than the second total applied electrical energy, and wherein the first total applied electrical energy is greater than the second total applied electrical energy. Aspect 58 is the method of Aspects 1 to 57, wherein the first and second field strengths of the first and second electrical pulses are a function of a voltage magnitude of the electrical pulses, duration of the electrical pulses, and a conductivity of the sample. Aspect 59 is the method of Aspects 1 to 58, wherein the first and second field strengths of the first and second electrical pulses are a function of a voltage magnitude of the electrical pulses, duration of the electrical pulses, and a conductivity of the sample, and wherein the voltage magnitude of the electrical pulses is between 0.001 Volts and 10,000 Volts, 0.01 Volts and 10,000 Volts, 0.1 Volts and 10,000 Volts, 1 Volt and 10,000 Volts, 1 Volt and 9,000 Volts, 1 Volt and 8,000 Volts, 1 Volt and 7,000 Volts, 1 Volt and 6,000 Volts, 1 Volt and 5,000 Volts, 1 Volt and 4,000 Volts, 1 Volt and 3,000 Volts, 1 Volt and 2,000 Volts, or 1 Volt and 1,000 Volts. Aspect 60 is the method of Aspects 1 to 59, wherein the first and second field strengths of the first and second electrical pulses are a function of a voltage magnitude of the electrical pulses, duration of the electrical pulses, and a conductivity of the sample, and wherein the voltage magnitude of the electrical pulses is between 100 Volts and 900 Volts. Aspect 61 is the method of Aspects 1 to 60, wherein the first and second field strengths of the first and second electrical pulses are a function of a voltage magnitude of the electrical pulses, duration of the electrical pulses, and a conductivity of the sample, and wherein the conductivity of the sample is a function of parameters comprising an ionic composition of electroporation buffer, concentration of an agent to be loaded into the cells, cell density, temperature, and pressure. Aspect 62 is the method of Aspects 1 to 61, wherein the first and second field strengths of the first and second electrical pulses are a function of a voltage magnitude of the electrical pulses, duration of the electrical pulses, and a conductivity of the sample, and wherein the conductivity of the sample is between 0.01 Siemens/meter and 10 Siemens/meter, 0.01 Siemens/meter and 1 Siemens/meter, 0.1 Siemens/meter and 10 Siemens/meter, 0.1 Siemens/meter and 1 Siemens/meter, or 1 Siemens/meter and 10 Siemens/meter. Aspect 63 is the method of Aspects 1 to 62, wherein the first and second field strengths of the first and second electrical pulses are a function of a voltage magnitude of the electrical pulses, duration of the electrical pulses, and a conductivity of the sample, and wherein the conductivity of the sample is between 1.0 and 3.0 Siemens/meter. Aspect 64 is the method of Aspects 1 to 63, wherein the first and second field strengths of the first and second electrical pulses are a function of a voltage magnitude of the electrical pulses, duration of the electrical pulses, and a conductivity of the sample, and wherein the first and second field strengths are further a function of a geometry of an electroporation chamber. Aspect 65 is the method of Aspects 1 to 64, wherein the first and second field strengths of the first and second electrical pulses are a function of a voltage magnitude of the electrical pulses, duration of the electrical pulses, and a conductivity of the sample, wherein the first and second field strengths are further a function of a geometry of an electroporation chamber, and wherein the electroporation chamber comprises an electrode gap between 0.001 cm and 10 cm, 0.001 cm and 1 cm, 0.01 cm and 10 cm, 0.01 cm and 1 cm, 0.1 cm and 10 cm, 0.1 cm and 1 cm, or 1 cm and 10 cm. Aspect 66 is the method of Aspects 1 to 65, wherein the first and second field strengths of the first and second electrical pulses are a function of a voltage magnitude of the electrical pulses, duration of the electrical pulses, and a conductivity of the sample, wherein the first and second field strengths are further a function of a geometry of an electroporation chamber, and wherein the electroporation chamber comprises an electrode gap between 0.01 cm and 1 cm. Aspect 67 is the method of Aspects 1 to 66, wherein the first and second field strengths of the first and second electrical pulses are between 0.01 kV/cm and 10 kV/cm, 0.01 kV/cm and 1 kV/cm, 0.1 kV/cm and 10 kV/cm, 0.1 kV/cm and 1 kV/cm, or 1 kV/cm and 10 kV/cm. Aspect 68 is the method of Aspects 1 to 67, wherein the first and second field strengths of the first and second electrical pulses are between 0.3 kV/cm and 3 kV/cm. Aspect 69 is the method of Aspects 1 to 68, wherein the first and second pulse durations of the first and second electrical pulses are between 10⁻⁶ seconds and 10 seconds, 10⁻⁶ seconds and 1 second, 10⁻³ seconds and 10 seconds, or 10⁻³ seconds and 1 second. Aspect 70 is the method of Aspects 1 to 69, wherein the first and second pulse durations of the first and second electrical pulses are between 1 microsecond and 100 milliseconds. Aspect 71 is the method of Aspects 1 to 70, wherein the first and second electrical pulses further comprise characteristics relating to pulse number, width, shape, pattern, or polarity. Aspect 72 is the method of Aspects 1 to 71, wherein the first and second electrical pulses further comprise characteristics relating to pulse number, width, shape, pattern, or polarity, and wherein the pulse number is between 1 pulse and 1000 pulses, 1 pulse and 900 pulses, 1 pulse and 800 pulses, 1 pulse and 700 pulses, 1 pulse and 600 pulses, 1 pulse and 500 pulses, 1 pulse and 400 pulses, 1 pulse and 300 pulses, 1 pulse and 200 pulses, 1 pulse and 100 pulses, 1 pulse and 90 pulses, 1 pulse and 80 pulses, 1 pulse and 70 pulses, 1 pulse and 60 pulses, 1 pulse and 50 pulses, 1 pulse and 40 pulses, 1 pulse and 30 pulses, 1 pulse and 20 pulses, or 1 pulse and 10 pulses. Aspect 73 is the method of Aspects 1 to 72, wherein the first and second electrical pulses further comprise characteristics relating to pulse number, width, shape, pattern, or polarity, and wherein the pulse number is between 1 pulse and 130 pulses. Aspect 74 is the method of Aspects 1 to 73, wherein the first and second electrical pulses further comprise characteristics relating to pulse number, width, shape, pattern, or polarity, and wherein the pulse width is a function of a rate of exponential decay. Aspect 75 is the method of Aspects 1 to 74, wherein the first and second electrical pulses further comprise characteristics relating to pulse number, width, shape, pattern, or polarity, wherein the pulse width is a function of a rate of exponential decay, and wherein the rate of exponential decay is a function of a resistance of the sample and a capacitance of a power supply used to effect electroporation. Aspect 76 is the method of Aspects 1 to 75, wherein the first and second electrical pulses further comprise characteristics relating to pulse number, width, shape, pattern, or polarity, wherein the pulse width is a function of a rate of exponential decay, wherein the rate of exponential decay is a function of a resistance of the sample and a capacitance of a power supply used to effect electroporation, and wherein resistance of the sample is between 1 ohm and 10000 ohms, 1 ohm and 9000 ohms, 1 ohm and 8000 ohms, 1 ohm and 7000 ohms, 1 ohm and 6000 ohms, 1 ohm and 5000 ohms, 1 ohm and 4000 ohms, 1 ohm and 3000 ohms, 1 ohm and 2000 ohms, 1 ohm and 1000 ohms, 1 ohm and 900 ohms, 1 ohm and 800 ohms, 1 ohm and 700 ohms, 1 ohm and 600 ohms, 1 ohm and 500 ohms, 1 ohm and 400 ohms, 1 ohm and 300 ohms, 1 ohm and 200 ohms, 1 ohm and 100 ohms, 1 ohm and 90 ohms, 1 ohm and 80 ohms, 1 ohm and 70 ohms, 1 ohm and 60 ohms, 1 ohm and 50 ohms, 1 ohm and 40 ohms, 1 ohm and 30 ohms, 1 ohm and 20 ohms, or 1 ohm and 10 ohms. Aspect 77 is the method of Aspects 1 to 76, wherein the first and second electrical pulses further comprise characteristics relating to pulse number, width, shape, pattern, or polarity, wherein the pulse width is a function of a rate of exponential decay, wherein the rate of exponential decay is a function of a resistance of the sample and a capacitance of a power supply used to effect electroporation, and wherein the resistance of the sample is between 1 ohm and 1000 ohms. Aspect 78 is the method of Aspects 1 to 77, wherein the first and second electrical pulses further comprise characteristics relating to pulse number, width, shape, pattern, or polarity, wherein the pulse width is a function of a rate of exponential decay, wherein the rate of exponential decay is a function of a resistance of the sample and a capacitance of a power supply used to effect electroporation, and wherein the power supply capacitance is between 1 μF and 1,000,000 μF, 1 μF and 100,000 μF, 1 μF and 10,000 μF, 1 μF and 1,000 μF, or 1 μF and 100 μF. Aspect 79 is the method of Aspects 1 to 78, wherein the first and second electrical pulses further comprise characteristics relating to pulse number, width, shape, pattern, or polarity, wherein the pulse width is a function of a rate of exponential decay, wherein the rate of exponential decay is a function of a resistance of the sample and a capacitance of a power supply used to effect electroporation, and wherein the power supply capacitance is between 1000 μF and 5000 μF. Aspect 80 is the method of Aspects 1 to 79, wherein the first and second electrical pulses further comprise characteristics relating to pulse number, width, shape, pattern, or polarity, and wherein the pulse shape is a square wave pulse or an exponential decay wave pulse. Aspect 81 is the method of Aspects 1 to 80, wherein the first and second electrical pulses further comprise characteristics relating to pulse number, width, shape, pattern, or polarity, and wherein the pulse pattern comprises a single pulse corresponding to the duration of the first or second pulse. Aspect 82 is the method of Aspects 1 to 81, wherein the first and second electrical pulses further comprise characteristics relating to pulse number, width, shape, pattern, or polarity, and wherein the pulse pattern comprises multiple pulses, wherein a combined duration of the multiple pulses corresponds to the duration of the first or second pulse. Aspect 83 is the method of Aspects 1 to 82, wherein the first and second electrical pulses further comprise characteristics relating to pulse number, width, shape, pattern, or polarity, and wherein the polarity of the first and second electrical pulses is positive or negative. Aspect 84 is the method of Aspects 1 to 83, wherein the sample is subjected to the second electrical pulse at least about 12 hours to at least about 48 hours after the sample is subjected to the first pulse. Aspect 85 is the method of Aspects 1 to 84, wherein the sample is subjected to the second electrical pulse at least about 24 hours after the sample is subjected to the first pulse. Aspect 86 is the method of Aspects 1 to 85, wherein the method is performed by an electroporation system having a non-transitory computer readable medium comprising instructions that, when executed by a processor, cause the processor to execute the first and second protocols to electroporate the sample. Aspect 87 is the method of Aspects 1 to 86, wherein the electroporation system comprises a flow electroporation apparatus. Aspect 88 is the method of Aspects 1 to 87, wherein the electroporation system comprises a flow electroporation apparatus, and wherein the sample is subjected to the electrical pulses while the sample is flowing within the flow electroporation apparatus. Aspect 89 is the method of Aspects 1 to 88, wherein the cells are mammalian cells. Aspect 90 is the method of Aspects 1 to 89, wherein the cells are human cells, murine cells, rat cells, hamster cells, or primate cells. Aspect 91 is the method of Aspects 1 to 90, wherein the cells are primary cells. Aspect 92 is the method of Aspects 1 to 91, wherein the cells are cultured cells. Aspect 93 is the method of Aspects 1 to 92, wherein the cells are cultured cells, and wherein cultured cells are cultured cell lines. Aspect 94 is the method of Aspects 1 to 93, wherein the cells are cultured cell lines, and wherein the cultured cell lines comprise 3T3, 697, 10T½, 1321N1, A549, AHR77, B-LCL, B16, B65, Ba/F3, BHK, C2C12, C6, CaCo-2AP, CaSki, ChaGo-K-1, CHO, COS, DG75, DLD-1, EL4, H1299, HaCaT, HAP1, HCT116, HEK, HeLa, HepG2, HL60, HOS, HT1080, HT29, Huh-7, HUVEC, INS-1/GRINCH, Jurkat, K46, K562, KG1, KHYG-1, L5278Y, L6, LNCaP, LS180, MCF7, MDA-MB-231, ME-180, MG-63, Min-6, MOLT4, Nalm6, ND7/23, Neuro2a, NK92, NS/0, P3U1, Panc-1, PC-3, PC12, PER.C6, PM1, Ramos, RAW 264.7, RBL, Renca, RLE, SH-SY5Y, SK-BR-3, SK-MES-1, SK-N-SH, SK-OV-3, SP2/0, SW403, THP-1, U2OS, U937, Vero, YB2/0, or derivatives thereof. Aspect 95 is the method of Aspects 1 to 94, wherein the cells comprise adipocytes, chondrocytes, endothelial cells, epithelial cells, fibroblasts, hepatocytes, keratinocytes, myocytes, neurons, osteocytes, peripheral blood mononuclear cells (PBMCs), splenocytes, stem cells, or thymocytes. Aspect 96 is the method of Aspects 1 to 95, wherein the cells comprise PBMCs, and wherein the PBMCs are peripheral blood lymphocytes (PBLs). Aspect 97 is the method of Aspects 1 to 95, wherein the cells comprise PBMCs, wherein the PBMCs comprise PBLs, and wherein the PBLs are natural killer (NK) cells, T cells, or B cells. Aspect 98 is the method of Aspects 1 to 95, wherein the cells comprise PBMCs, and wherein the PBMCs are monocytes. Aspect 99 is the method of Aspects 1 to 95, wherein the cells comprise PBMCs, wherein the PBMCs are monocytes, and wherein the monocytes are macrophages or dendritic cells. Aspect 100 is the method of Aspects 1 to 95, wherein the cells comprise PBMCs, wherein the PBMCs are monocytes, wherein the monocytes are macrophages or dendritic cells, and wherein the macrophages are microglia. Aspect 101 is the method of Aspects 1 to 95, wherein the cells comprise stem cells, and wherein the stem cells are adipose stem cells, embryonic stem cells, hematopoietic stem cells, induced pluripotent stem cells, mesenchymal stem cells, or neural stem cells. Aspect 102 is the method of Aspects 1 to 101, wherein a loading efficiency of the agent is at least 50, 60, 70, 80, or 90%. Aspect 103 is the method of Aspects 1 to 102, wherein cell viability is at least 50% 12 to 96 hours after the second electrical pulse. Aspect 104 is the method of Aspects 1 to 103, wherein cell viability is at least 60% 12 to 96 hours after the second electrical pulse. Aspect 105 is the method of Aspects 1 to 104, wherein cell viability is at least 70% 12 to 96 hours after the second electrical pulse. Aspect 106 is the method of Aspects 1 to 105, wherein cell viability is at least 80% 12 to 96 hours after the second electrical pulse. Aspect 107 is the method of Aspects 1 to 106, wherein cell viability is at least 90% 12 to 96 hours after the second electrical pulse. Aspect 108 is the method of Aspects 1 to 107, wherein the electroporated cells are approximately 50% to 90% viable 12 to 96 hours after the second electrical pulse. Aspect 109 is the method of Aspects 1 to 108, wherein the electroporated cells are approximately 50% to 90% viable 12 to 72 hours after the second electrical pulse. Aspect 110 is the method of Aspects 1 to 109, wherein the electroporated cells are approximately 50% to 90% viable 12 to 48 hours after the second electrical pulse. Aspect 111 is the method of Aspects 1 to 110, wherein the electroporated cells are approximately 50% to 90% viable 24 hours after the second electrical pulse. Aspect 112 is the method of Aspects 1 to 111, wherein the electroporated cells are approximately 60% to 90% viable 12 to 96 hours after the second electrical pulse. Aspect 113 is the method of Aspects 1 to 112, wherein the electroporated cells are approximately 60% to 90% viable 12 to 72 hours after the second electrical pulse. Aspect 114 is the method of Aspects 1 to 113, wherein the electroporated cells are approximately 60% to 90% viable 12 to 48 hours after the second electrical pulse. Aspect 115 is the method of Aspects 1 to 114, wherein the electroporated cells are approximately 60% to 90% viable 24 hours after the second electrical pulse.

Aspect 116 is an electroporated cell, cell particle, or lipid vesicle produced using the method of Aspects 1-115.

Aspect 117 is an electroporation system having a non-transitory computer readable medium comprising instructions that, when executed by a processor, cause the processor to: select a first protocol associated with a first electrical pulse having a first field strength and a first pulse duration; subject a sample comprising one or more intact cells, cell particles, or lipid vesicles to the first electrical pulse defined by the first protocol sufficient to load the cells, cell particles, or lipid vesicles with a first agent according to the first protocol; select a second protocol associated with a second electrical pulse having a second field strength and a second pulse duration; and subject the sample to the second electrical pulse defined by the second protocol sufficient to load the cells, cell particles, or lipid vesicles with a second agent according to the second protocol; wherein the first field strength and/or the first pulse duration are different from the second field strength and/or second pulse duration. Aspect 118 is the electroporation system of Aspect 117, wherein the first field strength equals the second field strength, and wherein the first pulse duration is longer than the second pulse duration. Aspect 119 is the electroporation system of Aspect 117, wherein the first field strength equals the second field strength, and wherein the first pulse duration is shorter than the second pulse duration. Aspect 120 is the electroporation system of Aspect 117, wherein the first field strength is less than the second field strength, and wherein the first pulse duration equals the second pulse duration. Aspect 121 is the electroporation system of Aspect 117, wherein the first field strength is greater than the second field strength, and wherein the first pulse duration equals the second pulse duration. Aspect 122 is the electroporation system of Aspect 117, wherein the first field strength is less than the second field strength, and wherein the first pulse duration is longer than the second pulse duration. Aspect 123 is the electroporation system of Aspect 117, wherein the first field strength is greater than the second field strength, and wherein the first pulse duration longer than the second pulse duration. Aspect 124 is the electroporation system of Aspect 117, wherein the first field strength is less than the second field strength, and wherein the first pulse duration is shorter than the second pulse duration. Aspect 125 is the electroporation system of Aspect 117, wherein the first field strength is greater than the second field strength, and wherein the first pulse duration is shorter than the second pulse duration. Aspect 126 is the electroporation system of Aspect 117 to Aspect 125, wherein the first field strength and first pulse duration produce a first total applied electrical energy and the second field strength and second pulse duration produce a second total applied electrical energy, and wherein the first total applied electrical energy is different than the second total applied electrical energy. Aspect 127 is the electroporation system of Aspect 117 to Aspect 126, wherein the first field strength and first pulse duration produce a first total applied electrical energy and the second field strength and second pulse duration produce a second total applied electrical energy, wherein the first total applied electrical energy is different than the second total applied electrical energy, and wherein the first total applied electrical energy is greater than the second total applied electrical energy. Aspect 128 is the electroporation system of Aspect 117 to Aspect 127, wherein the first and second field strengths of the first and second electrical pulses are a function of a voltage magnitude of the electrical pulses, duration of the electrical pulses, and a conductivity of the sample. Aspect 129 is the electroporation system of Aspect 117 to Aspect 128, wherein the first and second field strengths of the first and second electrical pulses are a function of a voltage magnitude of the electrical pulses, duration of the electrical pulses, and a conductivity of the sample, and wherein the voltage magnitude of the electrical pulses is between 0.001 Volts and 10,000 Volts, 0.01 Volts and 10,000 Volts, 0.1 Volts and 10,000 Volts, 1 Volt and 10,000 Volts, 1 Volt and 9,000 Volts, 1 Volt and 8,000 Volts, 1 Volt and 7,000 Volts, 1 Volt and 6,000 Volts, 1 Volt and 5,000 Volts, 1 Volt and 4,000 Volts, 1 Volt and 3,000 Volts, 1 Volt and 2,000 Volts, or 1 Volt and 1,000 Volts. Aspect 130 is the electroporation system of Aspect 117 to Aspect 129, wherein the first and second field strengths of the first and second electrical pulses are a function of a voltage magnitude of the electrical pulses, duration of the electrical pulses, and a conductivity of the sample, and wherein the voltage magnitude of the electrical pulses is between 100 Volts and 900 Volts. Aspect 131 is the electroporation system of Aspect 117 to Aspect 130, wherein the first and second field strengths of the first and second electrical pulses are a function of a voltage magnitude of the electrical pulses, duration of the electrical pulses, and a conductivity of the sample, and wherein the conductivity of the sample is a function of parameters comprising ionic composition of electroporation buffer, concentration of an agent to be loaded into the cells, cell density, temperature, and pressure. Aspect 132 is the electroporation system of Aspect 117 to Aspect 131, wherein the first and second field strengths of the first and second electrical pulses are a function of a voltage magnitude of the electrical pulses, duration of the electrical pulses, and a conductivity of the sample, and wherein the conductivity of the sample is between 0.01 Siemens/meter and 10 Siemens/meter, 0.01 Siemens/meter and 1 Siemens/meter, 0.1 Siemens/meter and 10 Siemens/meter, 0.1 Siemens/meter and 1 Siemens/meter, or 1 Siemens/meter and 10 Siemens/meter. Aspect 133 is the electroporation system of Aspect 117 to Aspect 132, wherein the first and second field strengths of the first and second electrical pulses are a function of a voltage magnitude of the electrical pulses, duration of the electrical pulses, and a conductivity of the sample, and wherein the conductivity of the sample is between 1.0 and 3.0 Siemens/meter. Aspect 134 is the electroporation system of Aspect 117 to Aspect 133, wherein the first and second field strengths of the first and second electrical pulses are a function of a voltage magnitude of the electrical pulses, duration of the electrical pulses, and a conductivity of the sample, and wherein the first and second field strengths are further a function of a geometry of an electroporation chamber. Aspect 135 is the electroporation system of Aspect 117 to Aspect 134, wherein the first and second field strengths of the first and second electrical pulses are a function of a voltage magnitude of the electrical pulses, duration of the electrical pulses, and a conductivity of the sample, wherein the first and second field strengths are further a function of a geometry of an electroporation chamber, and wherein the electroporation chamber comprises an electrode gap between 0.001 cm and 10 cm, 0.001 cm and 1 cm, 0.01 cm and 10 cm, 0.01 cm and 1 cm, 0.1 cm and 10 cm, 0.1 cm and 1 cm, or 1 cm and 10 cm. Aspect 136 is the electroporation system of Aspect 117 to Aspect 135, wherein the first and second field strengths of the first and second electrical pulses are a function of a voltage magnitude of the electrical pulses, duration of the electrical pulses, and a conductivity of the sample, wherein the first and second field strengths are further a function of a geometry of an electroporation chamber, and wherein the electroporation chamber comprises an electrode gap between 0.01 cm and 1 cm. Aspect 137 is the electroporation system of Aspect 117 to Aspect 136, wherein the first and second field strengths of the first and second electrical pulses are between 0.01 kV/cm and 10 kV/cm, 0.01 kV/cm and 1 kV/cm, 0.1 kV/cm and 10 kV/cm, 0.1 kV/cm and 1 kV/cm, or 1 kV/cm and 10 kV/cm. Aspect 138 is the electroporation system of Aspect 117 to Aspect 137, wherein the first and second field strengths of the first and second electrical pulses are between 0.3 kV/cm and 3 kV/cm. Aspect 139 is the electroporation system of Aspect 117 to Aspect 138, wherein the first and second pulse durations of the first and second electrical pulses are between 10⁻⁶ seconds and 10 seconds, 10⁻⁶ seconds and 1 second, 10⁻³ seconds and 10 seconds, or 10⁻³ seconds and 1 second. Aspect 140 is the electroporation system of Aspect 117 to Aspect 139, wherein the first and second pulse durations of the first and second electrical pulses are between 1 microsecond and 100 milliseconds. Aspect 141 is the electroporation system of Aspect 117 to Aspect 140, wherein the first and second electrical pulses further comprise characteristics relating to pulse number, width, shape, pattern, or polarity. Aspect 142 is the electroporation system of Aspect 117 to Aspect 141, wherein the first and second electrical pulses further comprise characteristics relating to pulse number, width, shape, pattern, or polarity, and wherein the pulse number is between 1 pulse and 1000 pulses, 1 pulse and 900 pulses, 1 pulse and 800 pulses, 1 pulse and 700 pulses, 1 pulse and 600 pulses, 1 pulse and 500 pulses, 1 pulse and 400 pulses, 1 pulse and 300 pulses, 1 pulse and 200 pulses, 1 pulse and 100 pulses, 1 pulse and 90 pulses, 1 pulse and 80 pulses, 1 pulse and 70 pulses, 1 pulse and 60 pulses, 1 pulse and 50 pulses, 1 pulse and 40 pulses, 1 pulse and 30 pulses, 1 pulse and 20 pulses, or 1 pulse and 10 pulses. Aspect 143 is the electroporation system of Aspect 117 to Aspect 142, wherein the first and second electrical pulses further comprise characteristics relating to pulse number, width, shape, pattern, or polarity, and wherein the pulse number is between 1 pulse and 130 pulses. Aspect 144 is the electroporation system of Aspect 117 to Aspect 143, wherein the first and second electrical pulses further comprise characteristics relating to pulse number, width, shape, pattern, or polarity, and wherein the pulse width is a function of a rate of exponential decay. Aspect 145 is the electroporation system of Aspect 117 to Aspect 144, wherein the first and second electrical pulses further comprise characteristics relating to pulse number, width, shape, pattern, or polarity, wherein the pulse width is a function of a rate of exponential decay, and wherein the rate of exponential decay is a function of a resistance of the sample and a capacitance of a power supply used to effect electroporation. Aspect 146 is the electroporation system of Aspect 117 to Aspect 145, wherein the first and second electrical pulses further comprise characteristics relating to pulse number, width, shape, pattern, or polarity, wherein the pulse width is a function of a rate of exponential decay, wherein the rate of exponential decay is a function of a resistance of the sample and a capacitance of a power supply used to effect electroporation, and wherein resistance of the sample is between 1 ohm and 10000 ohms, 1 ohm and 9000 ohms, 1 ohm and 8000 ohms, 1 ohm and 7000 ohms, 1 ohm and 6000 ohms, 1 ohm and 5000 ohms, 1 ohm and 4000 ohms, 1 ohm and 3000 ohms, 1 ohm and 2000 ohms, 1 ohm and 1000 ohms, 1 ohm and 900 ohms, 1 ohm and 800 ohms, 1 ohm and 700 ohms, 1 ohm and 600 ohms, 1 ohm and 500 ohms, 1 ohm and 400 ohms, 1 ohm and 300 ohms, 1 ohm and 200 ohms, 1 ohm and 100 ohms, 1 ohm and 90 ohms, 1 ohm and 80 ohms, 1 ohm and 70 ohms, 1 ohm and 60 ohms, 1 ohm and 50 ohms, 1 ohm and 40 ohms, 1 ohm and 30 ohms, 1 ohm and 20 ohms, or 1 ohm and 10 ohms. Aspect 147 is the electroporation system of Aspect 117 to Aspect 146, wherein the first and second electrical pulses further comprise characteristics relating to pulse number, width, shape, pattern, or polarity, wherein the pulse width is a function of a rate of exponential decay, wherein the rate of exponential decay is a function of a resistance of the sample and a capacitance of a power supply used to effect electroporation, and wherein resistance of the sample is between 1 ohm and 1000 ohms. Aspect 148 is the electroporation system of Aspect 117 to Aspect 147, wherein the first and second electrical pulses further comprise characteristics relating to pulse number, width, shape, pattern, or polarity, wherein the pulse width is a function of a rate of exponential decay, wherein the rate of exponential decay is a function of a resistance of the sample and a capacitance of a power supply used to effect electroporation, and wherein the power supply capacitance is between 1 μF and 1,000,000 μF, 1 μF and 100,000 μF, 1μF and10,000 μF, 1 μF and 1,000 μF, or 1 μF and 100 μF. Aspect 149 is the electroporation system of Aspect 117 to Aspect 148, wherein the first and second electrical pulses further comprise characteristics relating to pulse number, width, shape, pattern, or polarity, wherein the pulse width is a function of a rate of exponential decay, wherein the rate of exponential decay is a function of a resistance of the sample and a capacitance of a power supply used to effect electroporation, and wherein the power supply capacitance is between 1000 μF and 5000 μF. Aspect 150 is the electroporation system of Aspect 117 to Aspect 149, wherein the first and second electrical pulses further comprise characteristics relating to pulse number, width, shape, pattern, or polarity, wherein the pulse shape is a square wave pulse or an exponential decay wave pulse. Aspect 151 is the electroporation system of Aspect 117 to Aspect 150, wherein the first and second electrical pulses further comprise characteristics relating to pulse number, width, shape, pattern, or polarity, wherein the pulse pattern comprises a single pulse corresponding to the duration of the first or second pulse. Aspect 152 is the electroporation system of Aspect 117 to Aspect 150, wherein the pulse pattern comprises multiple pulses, wherein a combined duration of the multiple pulses corresponds to the duration of the first or second pulse. Aspect 153 is the electroporation system of Aspect 117 to Aspect 152, wherein the polarity of the first and second electrical pulses is positive or negative. Aspect 154 is the electroporation system of Aspect 117 to Aspect 153, wherein the sample is subjected to the second electrical pulse at least about 12 hours to at least about 48 hours after the sample is subjected to the first pulse. Aspect 155 is the electroporation system of Aspect 117 to Aspect 154, wherein the sample is subjected to the second electrical pulse at least about 24 hours after the sample is subjected to the first pulse. Aspect 156 is the electroporation system of Aspect 117 to Aspect 155, wherein the electroporation system comprises a flow electroporation apparatus. Aspect 157 is the electroporation system of Aspect 117 to Aspect 156, wherein the electroporation system comprises a flow electroporation apparatus, and wherein the sample is subjected to the electrical pulses while the sample is flowing within the flow electroporation apparatus. Aspect 158 is the electroporation system of Aspect 117 to Aspect 157, wherein the cells are mammalian cells. Aspect 159 is the electroporation system of Aspect 117 to Aspect 158, wherein the cells are human cells, murine cells, rat cells, hamster cells, or primate cells. Aspect 160 is the electroporation system of Aspect 117 to Aspect 159, wherein the cells are primary cells. Aspect 161 is the electroporation system of Aspect 117 to Aspect 160, wherein the cells are cultured cells. Aspect 162 is the electroporation system of Aspect 117 to Aspect 161, wherein the cells are cultured cells, and wherein the cultured cells are cultured cell lines. Aspect 163 is the electroporation system of Aspect 117 to Aspect 162, wherein the cells are cultured cell lines, and wherein the cultured cell lines comprise 3T3, 697, 10T½, 1321N1, A549, AHR77, B-LCL, B16, B65, Ba/F3, BHK, C2C12, C6, CaCo-2, CAP, CaSki, ChaGo-K-1, CHO, COS, DG75, DLD-1, EL4, H1299, HaCaT, HAP1, HCT116, HEK, HeLa, HepG2, HL60, HOS, HT1080, HT29, Huh-7, HUVEC, INS-1/GRINCH, Jurkat, K46, K562, KG1, KHYG-1, L5278Y, L6, LNCaP, LS180, MCF7, MDA-MB-231, ME-180, MG-63, Min-6, MOLT4, Nalm6, ND7/23, Neuro2a, NK92, NS/0, P3U1, Panc-1, PC-3, PC12, PER.C6, PM1, Ramos, RAW 264.7, RBL, Renca, RLE, SH-SY5Y, SK-BR-3, SK-MES-1, SK-N-SH, SK-OV-3, SP2/0, SW403, THP-1, U2OS, U937, Vero, YB2/0, or derivatives thereof. Aspect 164 is the electroporation system of Aspect 117 to Aspect 163, wherein the cells comprise adipocytes, chondrocytes, endothelial cells, epithelial cells, fibroblasts, hepatocytes, keratinocytes, myocytes, neurons, osteocytes, peripheral blood mononuclear cells (PBMCs), splenocytes, stem cells, or thymocytes. Aspect 165 is the electroporation system of Aspect 117 to Aspect 164, wherein the cells comprise PBMCs, and wherein the PBMCs are peripheral blood lymphocytes (PBLs). Aspect 166 is the electroporation system of Aspect 117 to Aspect 164, wherein the cells comprise PBMCs, wherein the PBMCs are PBLs, and wherein the PBLs are natural killer (NK) cells, T cells, or B cells. Aspect 167 is the electroporation system of Aspect 117 to Aspect 164, wherein the cells comprise PBMCs, and wherein the PBMCs are monocytes. Aspect 168 is the electroporation system of Aspect 117 to Aspect 164, wherein the cells comprise PBMCs, wherein the PBMCs are monocytes, and wherein the monocytes are macrophages or dendritic cells. Aspect 169 is the electroporation system of Aspect 117 to Aspect 164, wherein the cells comprise PBMCs, wherein the PBMCs are monocytes, wherein the monocytes are macrophages or dendritic cells, and wherein the macrophages are microglia. Aspect 170 is the electroporation system of Aspect 117 to Aspect 164, wherein the cells comprise stem cells, wherein the stem cells are adipose stem cells, embryonic stem cells, hematopoietic stem cells, induced pluripotent stem cells, mesenchymal stem cells, or neural stem cells. Aspect 171 is the electroporation system of Aspect 117 to Aspect 170, wherein the first and second agent are the same agent. Aspect 172 is the electroporation system of Aspect 117 to Aspect 170, wherein the first and second agent are different agents. Aspect 173 is the method of Aspects 117 to 172, wherein the first and second agents are a nucleic acid, polypeptide, protein, or small molecule. Aspect 174 is the method of Aspects 117 to 173, wherein the first agent is a nucleic acid, polypeptide, protein, or small molecule, and wherein the second agent is a nucleic acid, polypeptide, protein, or small molecule. Aspect 175 is the method of Aspects 117 to 174, wherein the first agent is a nucleic acid, and wherein the second agent is a nucleic acid, polypeptide, protein, or small molecule. Aspect 176 is the method of Aspects 117 to 174, wherein the first agent is a polypeptide, and wherein the second agent is a nucleic acid, polypeptide, protein, or small molecule. Aspect 177 is the method of Aspects 117 to 174, wherein the first agent is a protein, and wherein the second agent is a nucleic acid, polypeptide, protein, or small molecule. Aspect 178 is the method of Aspects 117 to 174, wherein the first agent is a small molecule, and wherein the second agent is a nucleic acid, polypeptide, protein, or small molecule. Aspect 179 is the method of Aspects 117 to 178, wherein the first agent is a nucleic acid, polypeptide, protein, or small molecule, and wherein the second agent is a nucleic acid. Aspect 180 is the method of Aspects 117 to 178, wherein the first agent is a nucleic acid, polypeptide, protein, or small molecule, and wherein the second agent is a polypeptide. Aspect 181 is the method of Aspects 117 to 178, wherein the first agent is a nucleic acid, polypeptide, protein, or small molecule, and wherein the second agent is a protein. Aspect 182 is the method of Aspects 117 to 178, wherein the first agent is a nucleic acid, polypeptide, protein, or small molecule, and wherein the second agent is a small molecule. Aspect 183 is the method of Aspects 117 to 182 wherein the nucleic acid is RNA, and wherein the RNA is mRNA, miRNA, shRNA, siRNA, or an antisense oligonucleotide. Aspect 184 is the method of Aspects 117 to 183, wherein the nucleic acid is DNA, and wherein the DNA is an antisense oligonucleotide, a vector, or a double sense linear DNA. Aspect 185 is the method of Aspects 117 to 184, wherein the protein is a ribonucleoprotein. Aspect 186 is the method of Aspects 117 to 185, wherein the ribonucleoprotein comprises a Cas9 protein and a guide RNA. Aspect 187 is the method of Aspects 117 to 186, wherein a loading efficiency of the agent is at least 50, 60, 70, 80, or 90%. Aspect 188 is the method of Aspects 117 to 187, wherein cell viability is at least 50% 12 to 96 hours after the second electrical pulse. Aspect 189 is the method of Aspects 117 to 188, wherein cell viability is at least 60% 12 to 96 hours after the second electrical pulse. Aspect 190 is the method of Aspects 117 to 189, wherein cell viability is at least 70% 12 to 96 hours after the second electrical pulse. Aspect 191 is the method of Aspects 117 to 190, wherein cell viability is at least 80% 12 to 96 hours after the second electrical pulse. Aspect 192 is the method of Aspects 117 to 191, wherein cell viability is at least 90% 12 to 96 hours after the second electrical pulse. Aspect 193 is the method of Aspects 117 to 192, wherein the electroporated cells are approximately 50% to 90% viable 12 to 96 hours after the second electrical pulse. Aspect 194 is the method of Aspects 117 to 193, wherein the electroporated cells are approximately 50% to 90% viable 12 to 72 hours after the second electrical pulse. Aspect 195 is the method of Aspects 117 to 194, wherein the electroporated cells are approximately 50% to 90% viable 12 to 48 hours after the second electrical pulse. Aspect 196 is the method of Aspects 117 to 195, wherein the electroporated cells are approximately 50% to 90% viable 24 hours after the second electrical pulse. Aspect 197 is the method of Aspects 117 to 196, wherein the electroporated cells are approximately 60% to 90% viable 12 to 96 hours after the second electrical pulse. Aspect 198 is the method of Aspects 117 to 197, wherein the electroporated cells are approximately 60% to 90% viable 12 to 72 hours after the second electrical pulse. Aspect 199 is the method of Aspects 117 to 198, wherein the electroporated cells are approximately 60% to 90% viable 12 to 48 hours after the second electrical pulse. Aspect 200 is the method of Aspects 117 to 199, wherein the electroporated cells are approximately 60% to 90% viable 24 hours after the second electrical pulse.

Aspect 201 is an electroporated cell, cell particle, or lipid vesicle produced using the electroporation system of Aspects 117 to 200.

Throughout this application, the term “cell” or “delivery vehicle” as it refers to a target of electroporation or a vehicle for delivery of an agent, drug, or therapeutic is meant to include human or animal cells in the biological sense.

As used herein, the term “energy” refers to the heat produced during an electrical pulse (or combined pulses) applied to a sample, and it is proportional to both the field strength and the pulse duration (or combined pulse duration) applied to the sample during the electrical pulse (or combined pulses). Thus, to apply a “high energy” pulse to a sample, the proportions of variables including field strength and pulse duration (or combined pulse duration) are modified such that a greater amount of heat is produced during the electrical pulse (or combined pulses) compared to when a “medium energy” or a “low energy” electrical pulse (or combined pulses) is applied to the sample, provided the buffer composition, the processing assembly, and the sample volume are held constant. Conversely, to apply a “low energy” pulse to a sample, the proportions of variables including field strength and pulse duration (or combined pulse duration) are modified such that a lesser amount of heat is produced during the electrical pulse (or combined pulses) compared to when a “high energy” or a “medium energy” electrical pulse (or combined pulses) is applied to the sample, provided the buffer composition, the processing assembly, and the sample volume are held constant.

Throughout this application, the terms “about,” “substantially,” and “approximately” are used to indicate that a value includes the inherent variation of error for the measurement or quantitation method or degree of variability in a value or range. Thus, the terms “about,” “substantially,” and “approximately” mean, in general, the stated value plus or minus 5%.

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristics of the disclosure. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that aspects described herein in the context of the term “comprising” may also be implemented in the context of the term “consisting of” or “consisting essentially of.”

It is specifically contemplated that any limitation discussed with respect to one aspect of the disclosure may apply to any other aspect of the disclosure. Furthermore, any composition of the disclosure may be used in any method of the disclosure, and any method of the disclosure may be used to produce or to utilize any composition of the disclosure. Aspects of an aspect set forth in the Examples are also aspects that may be implemented in the context of aspects discussed elsewhere in a different Example or elsewhere in the application, such as in the Summary, Detailed Description, Claims, and Brief Description of the Drawings.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIG. 1 illustrates a left, top perspective view of an electroporation processing assembly in a closed position, consistent with aspects of the present disclosure;

FIG. 2 illustrates a left, top perspective view of the processing assembly of FIG. 1 in an open position, consistent with aspects of the present disclosure;

FIG. 3 illustrates a rear, top, right perspective view of the processing assembly of FIG. 1 in the open position, consistent with aspects of the present disclosure;

FIG. 4 illustrates a rear, top, right perspective view of the processing assembly of FIG. 1 in the open position, consistent with aspects of the present disclosure;

FIG. 5 illustrates an exploded perspective view of the processing assembly of FIG. 4, consistent with aspects of the present disclosure;

FIG. 6 illustrates an exploded perspective view of the processing assembly of FIG. 4, consistent with aspects of the present disclosure;

FIG. 7 illustrates a top, right perspective view of the processing assembly of FIG. 1 with a label, consistent with aspects of the present disclosure;

FIG. 8 illustrates a top, left perspective view of the processing assembly of FIG. 1 with a label, consistent with aspects of the present disclosure;

FIG. 9 illustrates a top, right perspective view of the processing assembly of FIG. 1 with a loading device inserted, consistent with aspects of the present disclosure;

FIG. 10 illustrates a top, right perspective view of the processing assembly of FIG. 9, with portions of the processing assembly removed from view, consistent with aspects of the present disclosure;

FIG. 11 illustrates a top right perspective view of a tray holding electroporation processing assemblies, consistent with aspects of the present disclosure;

FIG. 12 illustrates a front view of trays holding electroporation processing assemblies, consistent with aspects of the present disclosure;

FIG. 13 illustrates a top right perspective view of a tray holding electroporation processing assemblies, consistent with aspects of the present disclosure;

FIG. 14 illustrates front views of a plurality of gaskets, consistent with aspects of the present disclosure;

FIG. 15 illustrates a top view of an array of gaskets and a front view of a gasket, consistent with aspects of the present disclosure;

FIG. 16 illustrates a front view of a bag and processing apparatus consistent with aspects of the present disclosure;

FIG. 17 illustrates a front view of a gasket, consistent with aspects of the present disclosure;

FIG. 18 illustrates a right, top perspective view of another electroporation processing assembly in a closed position, consistent with aspects of the present disclosure;

FIG. 19 illustrates a right, top perspective view of the processing assembly of FIG. 18 in an open position, consistent with aspects of the present disclosure;

FIG. 20 illustrates an exploded perspective view of the processing assembly of FIG. 18, consistent with aspects of the present disclosure;

FIG. 21 illustrates a tray holding a plurality of electroporation processing assemblies, consistent with aspects of the present disclosure;

FIG. 22 illustrates an electroporation processing assembly, consistent with aspects of the present disclosure;

FIG. 23 illustrates trays for holding a plurality of electroporation processing assemblies, consistent with aspects of the present disclosure;

FIG. 24 illustrates a tray for holding a plurality of electroporation processing assemblies, consistent with aspects of the present disclosure;

FIG. 25 illustrates a rack for holding a plurality of electroporation processing assemblies, consistent with aspects of the present disclosure;

FIG. 26 illustrates a rack for holding a plurality of electroporation processing assemblies, consistent with aspects of the present disclosure;

FIG. 27 illustrates electroporation systems, consistent with aspects of the present disclosure;

FIG. 28 illustrates a docking station in an open position with an electroporation processing assembly removed, consistent with aspects of the present disclosure;

FIG. 29 illustrates the docking station of FIG. 28 in an open position with a processing assembly inserted, consistent with aspects of the present disclosure;

FIG. 30 illustrates the docking station of FIG. 28 in a closed position with a processing assembly inserted, consistent with aspects of the present disclosure;

FIG. 31 illustrates a docking station in an open position, a closed position, and connected to an electroporation system, consistent with aspects of the present disclosure;

FIG. 32 illustrates a docking station connected to an electroporation system, consistent with aspects of the present disclosure;

FIG. 33 illustrates an electroporation device, processing assembly, docking station, trays, and a filling apparatus, consistent with aspects of this disclosure;

FIG. 34A-34C illustrates exemplary vessels for delivery to an electroporation system, consistent with aspects of the present disclosure.

FIG. 35 illustrates an experimental design for sequential electroporation of expanded lymphocytes with two different GFP mRNA concentrations (100 μg/mL and 200 μg/mL).

FIG. 36 shows flow cytometry data from Days 3 and 4 after sequential electroporation of expanded lymphocytes ells with GFP mRNA.

FIGS. 37A-37B show lymphocyte gating and viability of lymphocytes subjected to sequential electroporation.

FIGS. 38A-38B show GFP expression and GFP mean fluorescence intensity (MFI) for sequentially electroporated lymphocytes.

FIGS. 39A-39E illustrate experimental designs for sequential electroporation of expanded lymphocytes at different electroporation energies with two different GFP mRNA concentrations (100 μg/mL and 200 μg/mL).

FIGS. 40A-40B show populations of lymphocytes expressing GFP mRNA at three different time points (24 hr, 48 hr, and 72 hr) after sequential electroporation of expanded lymphocytes at different electroporation (EP) energies with two different GFP mRNA concentrations (100 μg/mL and 200 μg/mL).

FIGS. 41A-41B show that lymphocyte viability was comparable after sequential electroporation of expanded lymphocytes at different electroporation (EP) energies for all four energy combinations illustrated in FIGS. 39A-39E.

FIGS. 42A-42B show GFP expression by lymphocytes at three different time points (24 hr, 48 hr, and 72 hr) after sequential electroporation of expanded lymphocytes at different electroporation (EP) energies with two different GFP mRNA concentrations (100 μg/mL and 200 μg/mL).

FIGS. 43A-43B show GFP mean fluorescence intensity (MFI) for lymphocytes at three different time points (24 hr, 48 hr, and 72 hr) after sequential electroporation of expanded lymphocytes at different electroporation (EP) energies with two different GFP mRNA concentrations (100 μg/mL and 200 μg/mL).

FIGS. 44A-44B illustrate an experimental design, including cell culture (FIG. 44A) and electroporation (FIG. 44B) conditions, for sequential electroporation of activated T-cells with two different ribonucleoprotein (RNP) constructs to knock out TRAC and PD1.

FIG. 45 shows activation of T-cells after incubation with cytokines for 2 days.

FIGS. 46A-46F show a FACS gating strategy to measure TRAC and PD1 knockout efficiency in lymphocytes.

FIGS. 47A-47E show a FACS gating strategy to measure total cell and lymphocyte counts after electroporation with an RNP construct to knock out TRAC.

FIG. 48 is a schematic of one aspect of the present electroporation system.

FIG. 49 depicts an aspect of the present methods for subjecting a sample to two or more electrical pulses, which may be implemented using the electroporation system of FIG. 48.

### DETAILED DESCRIPTION

Certain aspects of the disclosure are directed to methods and apparatuses for sequential electroporation of cells, cell particles, lipid vesicles, liposomes, or tissues that provide for delivery of multiple rounds of electroporation separated in time to increase efficiency of entry of one or more agents of interest into cells, cell particles, lipid vesicles, liposomes, tissues, or derivatives thereof, and to minimize damage by electrical arc or heat shock.

### I. Electroporation

As used herein, electroporation or electroloading refers to application of an electrical current or electrical field to facilitate entry of an agent of interest into cells, cell particles, lipid vesicles, liposomes, tissues, or derivatives thereof. One of skill in the art will understand that any method and technique of electroporation is contemplated by the present disclosure.

The process of electroporation generally involves the formation of pores in a cell membrane, or in a vesicle or liposome, by the application of electric field pulses across a liquid cell suspension containing cells, vesicles, or liposomes. During the electroporation process, cells are often suspended in a liquid media and then subjected to an electric field pulse. The medium may be electrolyte, non-electrolyte, or a mixture of electrolytes and non-electrolytes. The strength of the electric field applied to the suspension and the length of the pulse (the time that the electric field is applied to a cell suspension) varies according to the cell type. To create a pore in a cell's outer membrane, the electric field must be applied for such a length of time and at such a voltage as to increase permeability of the cell membrane to allow an agent of interest to enter the cell.

Electroporation parameters may be adjusted to optimize the strength of the applied electrical field and/or duration of exposure such that the pores formed in membranes by the electrical pulse reseal after a short period of time, during which extracellular compounds have a chance to enter into the cell. However, excessive exposure of live cells to electrical fields can cause apoptosis and/or necrosis, which result in cell death. This is in part because, during an electroporation process, the electrical current flowing through a conductive media causes heating of the media and a subsequent increase in conductivity of the media. If not properly controlled, such a conductivity increase leads to even more current being drawn from a power source, which can lead to arcing and loss of sample. This effect is typically observed at relatively high field strengths (>2 kV/cm). However, electroporation of difficult-to-transfect cells, cell particles, lipid vesicles, liposomes, or tissues, for example, requires relatively strong electrical fields.

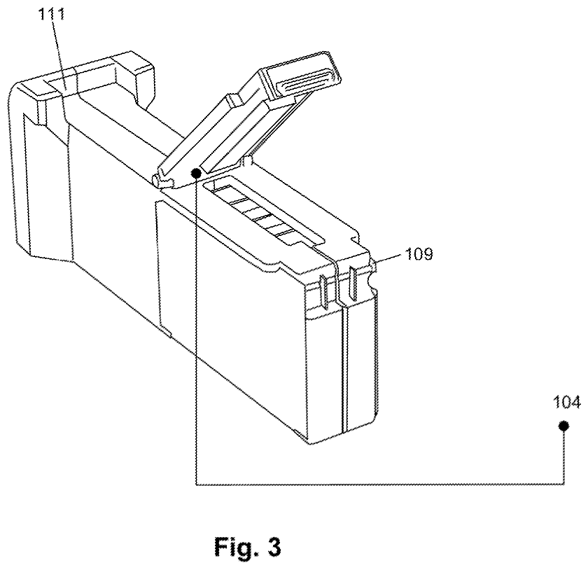

As an example, buffers developed for electroporation typically have relatively high conductivity, and very short electrical pulses are used. However, to efficiently load difficult-to-transfect cells or liposomes with an agent of interest, application of high voltages to highly conductive media for relatively long intervals of time may be required. These three conditions are not easily met concurrently, and doing so may result in irreversible damage to the cells or liposomes. Before conducting the experiments described herein, the inventors posited that multiple rounds of electroporation could provide for efficient loading of difficult-to-transfect cells or liposomes with an agent of interest. Currently, however, sequential electroporation is generally avoided since, as with applying high voltages to highly conductive media for relatively long intervals of time, multiple rounds of electroporation are known to irreversibly damage cells or liposomes and result in cell death. For example, O'Dea et al. (Vector-free intracellular delivery by reversible permeabilization. PLoS ONE. 2017; 12(3):e0174779) explicitly notes that the described vector-free reversible cell permeabilization methods make multiple dosing of genetic material to cells possible, in contrast to techniques such as electroporation for which multiple dosing is not possible because multiple rounds of electroporation result in cell death. In fact, Plews et al. (Activation of Pluripotency Genes in Human Fibroblast Cells by a Novel mRNA Based Approach. PLoS ONE. 2010; 5(12): e14397) actually demonstrated that attempting multiple rounds of electroporation caused massive cell death. Similarly, Rols and Teissie (Electropermeabilization of Mammalian Cells to Macromolecules: Control by Pulse Duration. Biophys. J. 1998; 75(3): 1415-1423) show at FIGS. 2A and 2C a dramatic decrease in cell viability with an increase in either pulse duration or number of pulses applied in sequence.

A solution to this problem involves the disclosed methods and apparatuses for electroporation that provide for sequential rounds of electroporation while minimizing damage to cells, cell particles, lipid vesicles, or tissues by electrical arc or a heat shock; increasing loading efficiency of an agent of interest; and maintaining viability of the cells, cell particles, lipid vesicles, or tissues and the ability of the cells, cell particles, lipid vesicles, liposomes, or tissues to produce a clinical effect. The inventors have developed electroporation methods comprising subjecting a sample comprising one or more intact cells, cell particles, or lipid vesicles to two or more electrical pulses having different electric field strengths and/or different pulse durations, and additionally, or alternatively, allowing the sample to recover for at least about 24 hours the two or more electrical pulses. The inventors surprisingly found that these electroporation methods can more efficiently load difficult-to-transfect samples with an agent of interest without reducing sample integrity or, for cell samples, cell viability, compared to previously described methods.

Some aspects include methods of encapsulating agents of interest; methods of transiently permeabilizing membranes to allow transport of agents of interest through the membranes; methods of electroporating cells, cell particles, lipid vesicles, liposomes, or tissues; methods of producing electroporated cells, cell particles, lipid vesicles, liposomes, or tissues; and methods of increasing efficiency of electroporation while maintaining clinical effect of electroporated materials. Some aspects also include an electroporated cell, cell particle, or lipid vesicle produced using any of the electroporation methods or apparatuses disclosed herein.

Some of the present methods include subjecting a sample comprising one or more intact cells, cell particles, or lipid vesicles to a first electrical pulse having a first field strength and a first pulse duration sufficient to load the cells, cell particles, or lipid vesicles with an agent according to a first protocol and subjecting the sample to a second electrical pulse having a second field strength and a second pulse duration sufficient to load the cells, cell particles, or lipid vesicles with an agent according to a second protocol. In some such methods, the first field strength and/or the first pulse duration are different from the second field strength and/or second pulse duration. Additionally, or alternatively, some methods include allowing the sample to recover for at least about 24 hours after subjecting the sample to the first electrical pulse.

In some aspects, the agent is a nucleic acid, polypeptide, protein, or small molecule. In some aspects, the nucleic acid is RNA, and the RNA is mRNA, miRNA, shRNA, siRNA, or an antisense oligonucleotide. In some aspects, the nucleic acid is DNA, and the DNA is an antisense oligonucleotide, a vector, or a double sense linear DNA. In some aspects, the protein is a ribonucleoprotein. In some aspects, the ribonucleoprotein comprises a Cas9 protein and a guide RNA.

In some aspects, methods disclosed herein are performed by an electroporation system having a non-transitory computer readable medium comprising instructions that, when executed by a processor, cause the processor to execute the first and second protocols to electroporate the sample. In some aspects, the electroporation system comprises a flow electroporation apparatus, and the sample is subjected to the electrical pulses while the sample is flowing within the flow electroporation apparatus.

In some aspects, the timing of the first and second electrical pulses and/or the applied electrical field and/or duration of exposure provided by the two or more electrical pulses may be adjusted such that damage to cells, cell particles, lipid vesicles, or tissues by electrical arc or a heat shock is minimized; loading efficiency of an agent of interest is increased; and viability of the cells, cell particles, lipid vesicles, or tissues and the ability of the cells, cell particles, lipid vesicles, liposomes, or tissues to produce a clinical effect is maintained.

In some aspects, the sample is allowed to rest after an electrical pulse (e.g., a first and/or second electrical pulse). In some aspects, the sample is rested after an electrical pulse for 10-30 minutes at 25-50° C. and 3-8% CO₂. Thus, in some aspects, the sample is rested after an electrical pulse for at most, at least, or about 10, 11, 12, 13, 14, 15, 16, 17, 18, 19, 20, 21, 22, 23, 24, 25, 26, 27, 28, 29, or 30 minutes, or any range or value derivable therein. In some aspects, the sample is rested after an electrical pulse at least, at most, or about 25, 26, 27, 28, 29, 30, 31, 32, 33, 34, 35, 36, 37, 38, 39, 40, 41, 42, 43, 44, 45, 46, 47, 48, 49, or 50° C., or any range or value derivable therein. In some aspects, the sample is rested after an electrical pulse at least, at most, or about 3, 4, 5, 6, 7, or 8% CO₂, or any range or value derivable therein. In specific aspects, the sample is rested after an electrical pulse for 20 minutes at 37° C. and 5% CO₂. In some aspects, the sample is not rested after an electrical pulse (e.g., a first and/or second electrical pulse).

In some aspects, the sample comprising one or more intact cells is allowed to recover by culturing the cells for at least, at most, or about 6, 12, 18, 24, 30, 36, 42, 48, 54, 60, 66, 72, 78, 84, 90, 96, 102, 108, 114, or 120 hours, or any range or value derivable therein, after the sample is subjected to the first pulse. In some aspects, the sample comprising one or more intact cells is allowed to recover by culturing the cells for at most or at least 6 hours to 120 hours, 6 hours to 96 hours, 6 hours to 72 hours, 6 hours to 48 hours, 6 hours to 24 hours, 6 hours to 12 hours, or any range or value derivable therein, after the sample is subjected to the first pulse. Thus, in some aspects, the sample is subjected to the second electrical pulse at least, at most, or about 6, 12, 18, 24, 30, 36, 42, 48, 54, 60, 66, 72, 78, 84, 90, 96, 102, 108, 114, or 120 hours, or any range or value derivable therein, after the sample is subjected to the first pulse. In some aspects, the sample is subjected to the second electrical pulse at most or at least 6 hours to 120 hours, 6 hours to 96 hours, 6 hours to 72 hours, 6 hours to 48 hours, 6 hours to 24 hours, 6 hours to 12 hours, or any range or value derivable therein, after the sample is subjected to the first pulse. In some aspects, the sample is subjected to the second electrical pulse between 6 hours and 120 hours, 6 hours and 96 hours, 6 hours and 72 hours, 6 hours and 48 hours, 6 hours and 24 hours, 6 hours and 12 hours, or any range or value derivable therein, after the sample is subjected to the first pulse. In some aspects, the sample is subjected to the second electrical pulse at least about 12 hours to at least about 48 hours after the sample is subjected to the first pulse. In some aspects, the sample is subjected to the second electrical pulse at least about 24 hours after the sample is subjected to the first pulse. In some aspects, the sample comprising one or more intact cells is not recovered by culturing the cells after the sample is subjected to the first pulse.

Recovering the sample or allowing the sample to recover means culturing the cells of the sample in any of the cell-culture vessels and cell culture media disclosed herein under conditions such as those disclosed herein that are appropriate and sufficient to facilitate restoration or return of the cells to an improved or desired state or condition. For example, recovery in culture may allow the cells to recover from the trauma of electroporation by, for instance, repairing cell walls, and to begin expressing or metabolizing the agent loaded into the cells upon electroporation of the cells.

With respect to field strength and pulse duration, in some aspects, the field strength of the first electrical pulse equals the field strength of the second electrical pulse, and the pulse duration of the first electrical pulse is longer than the pulse duration of the second electrical pulse. In some aspects, the field strength of the first electrical pulse equals the field strength of the second electrical pulse, and the pulse duration of the first electrical pulse is shorter than the pulse duration of the second electrical pulse. In some aspects, the field strength of the first electrical pulse equals the field strength of the second electrical pulse, and the pulse duration of the first electrical pulse equals the pulse duration of the second electrical pulse. In some aspects, the field strength of the first electrical pulse is greater than the field strength of the second electrical pulse, and the pulse duration of the first electrical pulse equals the pulse duration of the second electrical pulse. In some aspects, the field strength of the first electrical pulse is greater than the field strength of the second electrical pulse, and the pulse duration of the first electrical pulse is longer than the pulse duration of the second electrical pulse. In some aspects, the field strength of the first electrical pulse is greater than the field strength of the second electrical pulse, and the pulse duration of the first electrical pulse is shorter than the pulse duration of the second electrical pulse. In some aspects, the field strength of the first electrical pulse is less than the field strength of the second electrical pulse, and the pulse duration of the first electrical pulse equals the pulse duration of the second electrical pulse. In some aspects, the field strength of the first electrical pulse is less than the field strength of the second electrical pulse, and the pulse duration of the first electrical pulse is longer than the pulse duration of the second electrical pulse. In some aspects, the field strength of the first electrical pulse is less than the field strength of the second electrical pulse, and the pulse duration of the first electrical pulse is shorter than the pulse duration of the second electrical pulse.

In some aspects, the field strength of the first electrical pulse and pulse duration of the first electrical pulse produce a first total applied electrical energy, and the field strength of the second electrical pulse and pulse duration of the second electrical pulse produce a second total applied electrical energy. In some aspects, the first total applied electrical energy is different than the second total applied electrical energy. In some aspects, the first total applied electrical energy is greater than the second total applied electrical energy. In some aspects, the first total applied electrical energy is less than the second total applied electrical energy.

To achieve a first field strength and/or a first pulse duration that is equal to, less than, or greater than a second field strength and/or a second pulse duration, one or more electroporation variables or parameters can be optimized using the procedures and methods described herein. Aspects of the disclosure can be used in context with static and flow electroporation systems.

1. Electric Field Strength

Field strength is measured as the voltage delivered across an electrode gap and may be expressed as kV/cm. Field strength is critical to surpassing the electrical potential of the cell membrane to allow the temporary reversible permeation or pore formation to occur in the cell membrane, and the methods of the present disclosure are capable of subjecting the cells to a range of electric field strengths. In some aspects, the first and second field strengths of the first and second electrical pulses can be, be at least, or be at most 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 kV/cm, or any range or value derivable therein. In some aspects, the first and second field strengths of the first and second electrical pulses are at most or at least about 0.01 kV/cm to 10 kV/cm, 0.01 kV/cm to 1 kV/cm, 0.1 kV/cm to 10 kV/cm, 0.1 kV/cm to 1 kV/cm, 1 kV/cm to 10 kV/cm, or any value from 0.01 kV/cm to 10 kV/cm or range derivable therein. In some aspects, the first and second field strengths of the first and second electrical pulses are between 0.01 kV/cm and 10 kV/cm, 0.01 kV/cm and 1 kV/cm, 0.1 kV/cm and 10 kV/cm, 0.1 kV/cm and 1 kV/cm, 1 kV/cm and 10 kV/cm, or any value from 0.01 kV/cm to 10 kV/cm or range derivable therein. In some aspects, the first and second field strengths of the first and second electrical pulses are between 0.3 kV/cm and 3 kV/cm, any value from 0.3 kV/cm to 3 kV/cm, or any range or value derivable therein.

Field strength is a function of several factors, including voltage magnitude of an applied electrical pulse, duration of the electrical pulse, and conductivity of the sample being electroporated. Thus, in some aspects, the first and second field strengths of the first and second electrical pulses are a function of a voltage magnitude of the electrical pulses, duration of the electrical pulses, and a conductivity of the sample.

In some aspects, the voltage magnitude of the electrical pulses can be, be at least, or be at most 0.001, 0.010, 0.020, 0.030, 0.040, 0.050, 0.060, 0.070, 0.080, 0.090, 0.100, 0.110, 0.120, 0.130, 0.140, 0.150, 0.160, 0.170, 0.180, 0.190, 0.200, 0.210, 0.220, 0.230, 0.240, 0.250, 0.260, 0.270, 0.280, 0.290, 0.300, 0.310, 0.320, 0.330, 0.340, 0.350, 0.360, 0.370, 0.380, 0.390, 0.400, 0.410, 0.420, 0.430, 0.440, 0.450, 0.460, 0.470, 0.480, 0.490, 0.500, 0.510, 0.520, 0.530, 0.540, 0.550, 0.560, 0.570, 0.580, 0.590, 0.600, 0.610, 0.620, 0.630, 0.640, 0.650, 0.660, 0.670, 0.680, 0.690, 0.700, 0.710, 0.720, 0.730, 0.740, 0.750, 0.760, 0.770, 0.780, 0.790, 0.800, 0.810, 0.820, 0.830, 0.840, 0.850, 0.860, 0.870, 0.880, 0.890, 0.900, 0.910, 0.920, 0.930, 0.940, 0.950, 0.960, 0.970, 0.980, 0.990, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 Volts, or any range or value derivable therein. In some aspects, the voltage magnitude of the electrical pulses is at most or at least about 0.001 Volts to 10,000 Volts, 0.01 Volts to 10,000 Volts, 0.1 Volts to 10,000 Volts, 1 Volt to 10,000 Volts, 1 Volt to 9,000 Volts, 1 Volt to 8,000 Volts, 1 Volt to 7,000 Volts, 1 Volt to 6,000 Volts, 1 Volt to 5,000 Volts, 1 Volt to 4,000 Volts, 1 Volt to 3,000 Volts, 1 Volt to 2,000 Volts, 1 Volt to 1,000 Volts, or any value from 0.001 Volts to 10,000 Volts or range derivable therein. In some aspects, the voltage magnitude of the electrical pulses is between 0.001 Volts and 10,000 Volts, 0.01 Volts and 10,000 Volts, 0.1 Volts and 10,000 Volts, 1 Volt and 10,000 Volts, 1 Volt and 9,000 Volts, 1 Volt and 8,000 Volts, 1 Volt and 7,000 Volts, 1 Volt and 6,000 Volts, 1 Volt and 5,000 Volts, 1 Volt and 4,000 Volts, 1 Volt and 3,000 Volts, 1 Volt and 2,000 Volts, 1 Volt and 1,000 Volts, or any value from 0.001 Volts to 10,000 Volts or range derivable therein. In some aspects, the voltage magnitude of the electrical pulses is between 100 Volts and 900 Volts, any value from 100 Volts to 900 Volts, or any range or value derivable therein.

In some aspects, the conductivity of the sample is a function of parameters comprising an ionic composition of electroporation buffer, concentration of an agent to be loaded into the cells, cell density, temperature, and pressure. In some aspects, the conductivity of the sample can be, be at least, or be at most 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 Siemens/meter, or any range or value derivable therein. In some aspects, the conductivity of the sample is at most or at least about 0.01 Siemens/meter to 10 Siemens/meter, 0.01 Siemens/meter to 1 Siemens/meter, 0.1 Siemens/meter to 10 Siemens/meter, 0.1 Siemens/meter to 1 Siemens/meter, 1 Siemens/meter to 10 Siemens/meter, or any value from 0.01 Siemens/meter to 10 Siemens/meter or range derivable therein. In some aspects, the conductivity of the sample is between 0.01 Siemens/meter and 10 Siemens/meter, 0.01 Siemens/meter and 1 Siemens/meter, 0.1 Siemens/meter and 10 Siemens/meter, 0.1 Siemens/meter and 1 Siemens/meter, 1 Siemens/meter and 10 Siemens/meter, or any value from 0.01 Siemens/meter to 10 Siemens/meter or range derivable therein. In some aspects, the conductivity of the sample is between 1.0 and 3.0 Siemens/meter, any value from 1.0 Siemens/meter to 3.0 Siemens/meter, or any range or value derivable therein.

The ionic composition of a buffer used for electroporation can vary depending on the cell type. For example, highly conductive buffers such as PBS (Phosphate Buffered Saline <30 ohms) and HBSS (Hepes Buffer <30 ohms) or standard culture media, which may contain serum, may be used. Other buffers include hypoosmolar buffers in which cells absorb water shortly before an electrical pulse, which can result in cell swelling and can lower the optimal permeation voltage while ensuring the membrane is more easily permeable. Cells requiring the use of high resistance buffers (>3000 ohms) may require preparation and washing of the cells to remove excess salt ions to reduce the chance of arcing and sample loss. Ionic strength of an electroporation buffer has a direct effect on the resistance of the sample, which in turn affects the pulse length or time constant of the pulse. The volume of liquid in contact with an electrode also has significant effect on sample resistance for ionic solutions, and the resistance of the sample is inversely proportional to the volume of solution and pH. As volume increases, resistance decreases, which increases the probability of arcing and sample loss, while lowering the volume increases the resistance and decreases arc potential.

The size and concentration of an agent will have an effect on the electrical parameters used to transfect the cell. Smaller molecules (for example, siRNA or miRNA) may need higher voltages with microsecond pulse lengths, while larger molecules (for example, DNA and proteins) may need lower voltages with longer pulse lengths. The concentration of an oligonucleotide during an electroporation procedure may be from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 50, 75, 100, 150, 200, 250, 300 to about 350, 400, 500, 1000, 1500, 2000, 3000, 4000, or 5000 μg/mL, or any value from 0.01 μg/mL to 5000 μg/mL or range derivable therein. In certain aspects, the concentration of the oligonucleotide is at least 1 μg/mL. In further aspects, the concentration of the oligonucleotide is at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 150, 125, 150, 175, 200, 225, 250, 275, or 300 μg/mL, or any value from 1 μg/mL to 300 μg/mL or range derivable therein. The concentration of a polypeptide during an electroporation procedure may be from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 50, 75, 100, 150, 200, 250, 300 to about 350, 400, 500, 1000, 1500, 2000, 3000, 4000, or 5000 μg/mL, or any value from 0.01 μg/mL to 5000 μg/mL or range derivable therein. In certain aspects, the concentration of the polypeptide is at least 1 μg/mL. In further aspects, the concentration of the polypeptide is at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 μg/mL, or any value from 1 μg/mL to 300 μg/mL or range derivable therein.

Cell density can be related to cell size. Generally, smaller cell sizes require higher voltages while larger cell sizes require lower voltages for successful cell membrane permeation.

The temperature at which cells are maintained during electroporation can affect the efficiency of the electroporation. Samples pulsed at high voltage or exposed to multiple pulses and long pulse durations can cause sample heating, which can contribute to increased cell death and lower transfection efficiency. Maintaining the sample at a lower temperature can diminish the effects of overheating on cell viability and efficiency. In general, the standard pulse voltage used for cells at room temperature should be approximately doubled for electroporation at 4° C. in order to effectively permeate the cell membrane.

In some aspects, the geometry of an electroporation chamber may be adjusted to adjust electric field strength. Field strength is calculated using voltage divided by gap size. The geometry of an electroporation chamber can be a function of the distance between electrodes, or “gap size.” Thus, in some aspects, gap size of electrodes within an electroporation chamber may be controlled to adjust the electric field strength. By increasing the gap size, field strength can be increased without changing voltage. To derive the voltage needed to accomplish electroporation if the desired field strength and gap size are known, field strength (kV) is multiplied by gap size (cm). Electrodes of electroporation chambers can comprise two or more “plate” electrodes. The electrode plates can comprise any useful biocompatible and conductive material, including aluminum, titanium, and gold. The electrode plate can be addressable with an electric pulse as determined by the present disclosure. The electrodes can comprise an array of between 1 and 100 cathodes and 1 and 100 anodes, there being an even number of cathodes and anodes so as to form pairs of positive and negative electrodes. The plates can comprise a width dimension that is generally greater than the distance, or gap, between opposing electrodes, or greater than twice the gap distance.

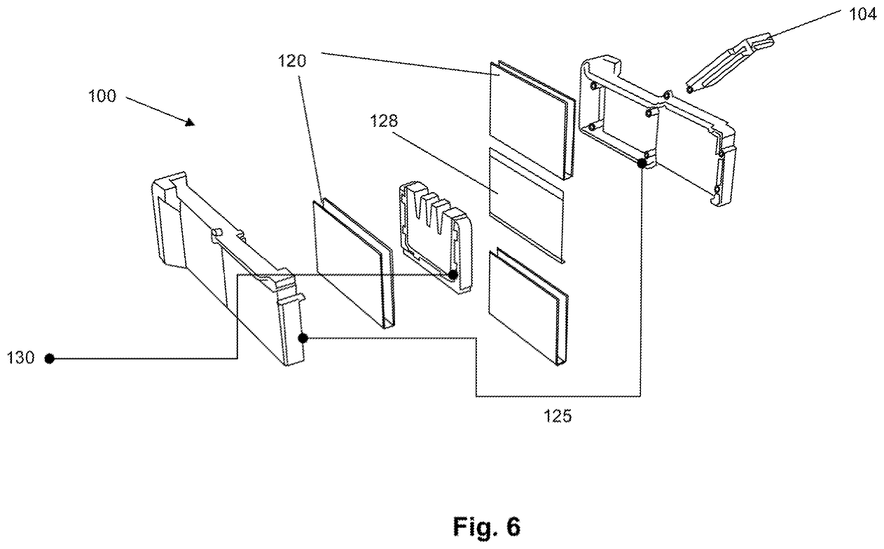

The cathode and anode electrodes can be spaced on opposing interior sides of an electroporation chamber such that the electroporation chamber comprises an electrode gap size of at least, at most, or about 0.001, 0.010, 0.020, 0.030, 0.040, 0.050, 0.060, 0.070, 0.080, 0.090, 0.100, 0.110, 0.120, 0.130, 0.140, 0.150, 0.160, 0.170, 0.180, 0.190, 0.200, 0.210, 0.220, 0.230, 0.240, 0.250, 0.260, 0.270, 0.280, 0.290, 0.300, 0.310, 0.320, 0.330, 0.340, 0.350, 0.360, 0.370, 0.380, 0.390, 0.400, 0.410, 0.420, 0.430, 0.440, 0.450, 0.460, 0.470, 0.480, 0.490, 0.500, 0.510, 0.520, 0.530, 0.540, 0.550, 0.560, 0.570, 0.580, 0.590, 0.600, 0.610, 0.620, 0.630, 0.640, 0.650, 0.660, 0.670, 0.680, 0.690, 0.700, 0.710, 0.720, 0.730, 0.740, 0.750, 0.760, 0.770, 0.780, 0.790, 0.800, 0.810, 0.820, 0.830, 0.840, 0.850, 0.860, 0.870, 0.880, 0.890, 0.900, 0.910, 0.920, 0.930, 0.940, 0.950, 0.960, 0.970, 0.980, 0.990, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 cm, or any range or value derivable therein. The cathode and anode electrodes can be spaced on opposing interior sides of an electroporation chamber such that the electroporation chamber comprises an electrode gap size of at most or at least about 0.001 cm to 10 cm, 0.001 cm to 1 cm, 0.01 cm to 10 cm, 0.01 cm to 1 cm, 0.1 cm to 10 cm, 0.1 cm to 1 cm, 1 cm to 10 cm, or any value from 0.001 cm to 10 cm or range derivable therein. FIG. 5 shows an electroporation chamber 108 formed, in some aspects, by opposing aluminum electrode buses 120 positioned around electroporation chamber 108 and surrounding a gasket 130 within chamber 108; the electrode gap comprises the thickness of the gasket 130, the thickness of the gasket corresponding to a side of the gasket 130 extending between the opposing electrode buses 120. In aspects in which the electroporation chamber 108 is formed by an electrode bus 120 and a gold-coated plastic film 128 that is positioned opposite to the opposing electrode bus 120 such that the gold-coated plastic film 128 is interposed between the opposing electrode buses 120, the thickness of the gasket comprising the electrode gap corresponds to a side of the gasket 130 extending between an electrode bus 120 and the gold-coated plastic film 128 that is positioned opposite to the opposing electrode bus 120. In some aspects, the electroporation chamber comprises an electrode gap that can be, be at least, or be at most 0.001, 0.010, 0.020, 0.030, 0.040, 0.050, 0.060, 0.070, 0.080, 0.090, 0.100, 0.110, 0.120, 0.130, 0.140, 0.150, 0.160, 0.170, 0.180, 0.190, 0.200, 0.210, 0.220, 0.230, 0.240, 0.250, 0.260, 0.270, 0.280, 0.290, 0.300, 0.310, 0.320, 0.330, 0.340, 0.350, 0.360, 0.370, 0.380, 0.390, 0.400, 0.410, 0.420, 0.430, 0.440, 0.450, 0.460, 0.470, 0.480, 0.490, 0.500, 0.510, 0.520, 0.530, 0.540, 0.550, 0.560, 0.570, 0.580, 0.590, 0.600, 0.610, 0.620, 0.630, 0.640, 0.650, 0.660, 0.670, 0.680, 0.690, 0.700, 0.710, 0.720, 0.730, 0.740, 0.750, 0.760, 0.770, 0.780, 0.790, 0.800, 0.810, 0.820, 0.830, 0.840, 0.850, 0.860, 0.870, 0.880, 0.890, 0.900, 0.910, 0.920, 0.930, 0.940, 0.950, 0.960, 0.970, 0.980, 0.990, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 cm, or any range or value derivable therein. In some aspects, the electroporation chamber comprises an electrode gap between 0.001 cm and 10 cm, 0.001 cm and 1 cm, 0.01 cm and 10 cm, 0.01 cm and 1 cm, 0.1 cm and 10 cm, 0.1 cm and 1 cm, 1 cm and 10 cm, or any value from 0.001 cm to 10 cm or range derivable therein. In some aspects, the electroporation chamber comprises an electrode gap between 0.01 cm and 1 cm, any value from 0.01 cm to 1 cm, or any range derivable therein. In some aspects, the electroporation chamber comprises an electrode gap between 0.4 cm and 1 cm, any value from 0.4 cm to 1 cm, or any range derivable therein. Each pair of said anodes and cathodes can be energized at a load resistance (in Ohms) depending upon the chamber size.

2. Electrical Pulse Characteristics

Pulse duration, or pulse length, is the duration of time the sample is exposed to an electrical pulse and is typically measured as time in micro to milliseconds ranges. The pulse length works indirectly with the field strength to increase pore formation and therefore the uptake of target molecules. Generally, an increase in voltage should be followed by an incremental decrease in pulse length. Decreasing the voltage, the reverse is true.

First and second pulse durations of the first and second electrical pulses to which the samples described herein are subjected can be, can be at least, or can be at most 10⁻⁶, 10⁻⁵, 10⁴, 10⁻³, 10⁻², 10⁻¹, 1, 2, 3, 4, 5, 6, 7, 8, 9, or 10 seconds, or any range or value derivable therein. First and second pulse durations of the first and second electrical pulses to which the samples described herein are subjected can be at most or at least 10⁻⁶ seconds to 10 seconds, 10⁻⁶ seconds to 1 second, 10⁻³ seconds to 10 seconds, 10⁻³ seconds to 1 second, or any range or value derivable therein. In some aspects, first and second pulse durations of the first and second electrical pulses are between 10⁻⁶ seconds and 10 seconds, 10⁻⁶ seconds and 1 second, 10⁻³ seconds and 10 seconds, 10⁻³ seconds and 1 second, or any value from 10⁻⁶ seconds to 10 seconds or range derivable therein. In some aspects, first and second pulse durations of the first and second electrical pulses are between 1 microsecond and 100 milliseconds, any value from 1 microsecond to 100 milliseconds, or any range derivable therein. In some aspects, first and second pulse durations of the first and second electrical pulses are between 6 microseconds and 65 milliseconds, any value from 6 microseconds to 65 milliseconds, or any range derivable therein.

In addition to pulse duration, electrical pulses can also be characterized by pulse number, pulse width, pulse shape, pulse pattern, and pulse polarity. Thus, in some aspects, the first and second electrical pulses further comprise characteristics relating to pulse number, pulse width, pulse shape, pulse pattern, or pulse polarity.

Electroporation can be carried out as a single pulse or as multiple pulses as disclosed herein to achieve maximum transfection efficiencies. In some aspects, pulse number can be, be at least, or be at most 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 pulses, or any range derivable therein. In some aspects, pulse number can be at most or at least 1 pulse to 1000 pulses, 1 pulse to 900 pulses, 1 pulse to 800 pulses, 1 pulse to 700 pulses, 1 pulse to 600 pulses, 1 pulse to 500 pulses, 1 pulse to 400 pulses, 1 pulse to 300 pulses, 1 pulse to 200 pulses, 1 pulse to 100 pulses, 1 pulse to 90 pulses, 1 pulse to 80 pulses, 1 pulse to 70 pulses, 1 pulse to 60 pulses, 1 pulse to 50 pulses, 1 pulse to 40 pulses, 1 pulse to 30 pulses, 1 pulse to 20 pulses, 1 pulse to 10 pulses, or any value from 1 pulse to 1000 pulses or range derivable therein. In some aspects, the pulse number is between 1 pulse and 1000 pulses, 1 pulse and 900 pulses, 1 pulse and 800 pulses, 1 pulse and 700 pulses, 1 pulse and 600 pulses, 1 pulse and 500 pulses, 1 pulse and 400 pulses, 1 pulse and 300 pulses, 1 pulse and 200 pulses, 1 pulse and 100 pulses, 1 pulse and 90 pulses, 1 pulse and 80 pulses, 1 pulse and 70 pulses, 1 pulse and 60 pulses, 1 pulse and 50 pulses, 1 pulse and 40 pulses, 1 pulse and 30 pulses, 1 pulse and 20 pulses, or 1 pulse and 10 pulses, or any value from 1 pulse to 1000 pulses or range derivable therein. In some aspects, the pulse number is between 1 and 130 pulses, any value from 1 to 130 pulses, or any range derivable therein.

Pulse width depends on the wave shape generated by a pulse generator of an electroporation system. Pulse shape, or wave form, generally falls into two categories, square wave or exponential decay wave. Square wave pulses rise quickly to a set voltage level and maintain this level during the duration of the set pulse length before quickly turning off. In some aspects, the pulse generator generates a square wave pulse, and pulse width can be inputted directly. Exponential decay waves generate an electrical pulse by allowing a capacitor to completely discharge. A pulse is discharged into a sample, and the voltage rises rapidly to the peak voltage set then declines over time. In some aspects, the pulse generator generates an exponential decay wave pulse, and the pulse width is a function of a rate of exponential decay.

The pulse width in an exponential decay wave system corresponds to the time constant and is characterized by the rate at which the pulsed energy or voltage is decayed to 1/3 the original set voltage. The time constant is modified by adjusting the resistance and capacitance values in an exponential decay, and the calculation for the time is T=RC, where T is time and R is resistance of a sample and C is capacitance of an electroporation system power supply. Thus, in some aspects, the rate of exponential decay is a function of a resistance of the sample and the capacitance of a power supply used to effect electroporation.

The resistance of a sample can be, can be at least, or can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 ohms, or any range or value derivable therein. The resistance of a sample can be at most or at least 1 ohm to 10000 ohms, 1 ohm to 9000 ohms, 1 ohm to 8000 ohms, 1 ohm to 7000 ohms, 1 ohm to 6000 ohms, 1 ohm to 5000 ohms, 1 ohm to 4000 ohms, 1 ohm to 3000 ohms, 1 ohm to 2000 ohms, 1 ohm to 1000 ohms, 1 ohm to 900 ohms, 1 ohm to 800 ohms, 1 ohm to 700 ohms, 1 ohm to 600 ohms, 1 ohm to 500 ohms, 1 ohm to 400 ohms, 1 ohm to 300 ohms, 1 ohm to 200 ohms, 1 ohm to 100 ohms, 1 ohm to 90 ohms, 1 ohm to 80 ohms, 1 ohm to 70 ohms, 1 ohm to 60 ohms, 1 ohm to 50 ohms, 1 ohm to 40 ohms, 1 ohm to 30 ohms, 1 ohm to 20 ohms, 1 ohm to 10 ohms, or any value from 1 ohm to 10000 ohms or range derivable therein. In some aspects, the resistance of the sample is between 1 ohm and 10000 ohms, 1 ohm and 9000 ohms, 1 ohm and 8000 ohms, 1 ohm and 7000 ohms, 1 ohm and 6000 ohms, 1 ohm and 5000 ohms, 1 ohm and 4000 ohms, 1 ohm and 3000 ohms, 1 ohm and 2000 ohms, 1 ohm and 1000 ohms, 1 ohm and 900 ohms, 1 ohm and 800 ohms, 1 ohm and 700 ohms, 1 ohm and 600 ohms, 1 ohm and 500 ohms, 1 ohm and 400 ohms, 1 ohm and 300 ohms, 1 ohm and 200 ohms, 1 ohm and 100 ohms, 1 ohm and 90 ohms, 1 ohm and 80 ohms, 1 ohm and 70 ohms, 1 ohm and 60 ohms, 1 ohm and 50 ohms, 1 ohm and 40 ohms, 1 ohm and 30 ohms, 1 ohm and 20 ohms, 1 ohm and 10 ohms, or any value from 1 ohm to 10000 ohms or range derivable therein. In some aspects, the resistance of the sample is between 1 ohm and 1000 ohms, any value from 1 ohm to 1000 ohms, or any range derivable therein.

The power supply capacitance can be, can be at least, or can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, 700000, 710000, 720000, 730000, 740000, 750000, 760000, 770000, 780000, 790000, 800000, 810000, 820000, 830000, 840000, 850000, 860000, 870000, 880000, 890000, 900000, 910000, 920000, 930000, 940000, 950000, 960000, 970000, 980000, 990000, or 1000000 μF, or any range or value derivable therein. The power supply capacitance can be at most or at least 1 μF to 1,000,000 μF, 1 μF to 100,000 μF, 1 μF to 10,000 μF, 1 μF to 1,000 μF, 1 μF to 100 μF, or any value from 1 μF to 1,000,000 μF or range derivable therein. In some aspects, the power supply capacitance is between 1 μF and 1,000,000 μF, 1 μF and 100,000 μF, 1 μF and 10,000 μF, 1 μF and 1,000 μF, 1 μF and 100 μF, or any value from 1 μF to 1,000,000 μF or range derivable therein. In some aspects, the power supply capacitance is between 1000 μF and 5000 μF, any value from 1000 μF to 5000 μF, or any range derivable therein.

In some aspects, the pulse pattern comprises a single pulse corresponding to the duration of the first and/or second pulse. In some aspects, the pulse pattern comprises multiple pulses, and a combined duration of the multiple pulses corresponds to the duration of the first and/or second pulse. Thus, in some aspects, pulse duration is the result of the additive effect of multiple pulses.

The polarity of the first and second electrical pulses to which the samples described herein may be subjected can be positive or negative. In some aspects, the polarity of the first and second pulses is positive. In some aspects, the polarity of the first and second pulses is negative. In some aspects, the polarity of the first pulse is positive, and the polarity of the second pulse is negative. In some aspects, the polarity of the first pulse is negative, and the polarity of the second pulse is positive.

In certain aspects, electroloading may be carried out as described in U.S. Pat. No. 5,612,207 (specifically incorporated herein by reference), U.S. Pat. No. 5,720,921 (specifically incorporated herein by reference), U.S. Pat. No. 6,074,605 (specifically incorporated herein by reference); U.S. Pat. No. 6,090,617 (specifically incorporated herein by reference); U.S. Pat. No. 6,485,961 (specifically incorporated herein by reference); U.S. Pat. No. 7,029,916 (specifically incorporated herein by reference), U.S. Pat. No. 7,141,425 (specifically incorporated herein by reference), U.S. Pat. No. 7,186,559 (specifically incorporated herein by reference), U.S. Pat. No. 7,771,984 (specifically incorporated herein by reference), and/or U.S. publication number 2011/0065171 (specifically incorporated herein by reference).

Other methods and devices for electroloading that may be used in the context of the present disclosure are also described in, for example, published PCT Application Nos. WO 03/018751 and WO 2004/031353; U.S. patent application Ser. Nos. 2004/0214333 and 2004/0115784; and U.S. Pat. Nos. 6,773,669, 6,090,617, 6,617,154, and 7,029,916, all of which are incorporated herein by reference.

In certain aspects of the disclosure, electroporation may be carried out as described in U.S. Pat. No. 7,141,425, granted Nov. 28, 2006, the entire disclosure of which is specifically incorporated herein by reference.

### II. Description of Representative Electroporation Apparatus

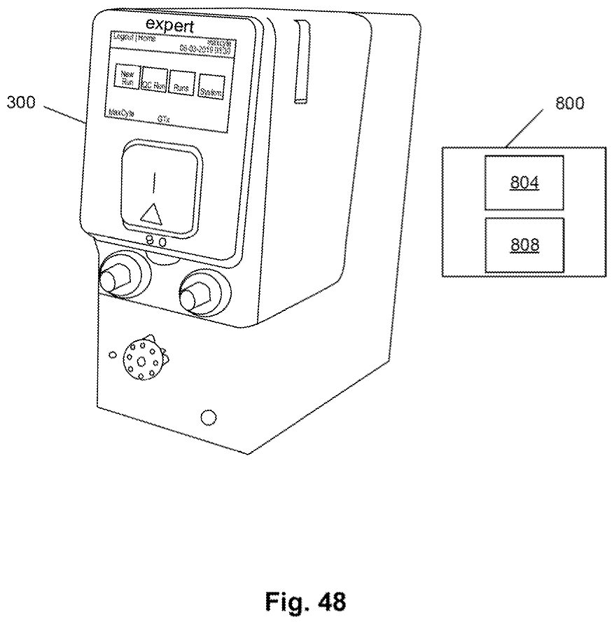

As shown in FIG. 48, some aspects of the present disclosure may also include an electroporation system 300 that includes a device (e.g., a controller, 800) and a non-transitory computer readable medium (e.g., one or more storage devices, 804) comprising (e.g., storing) instructions, that when executed by a processor 808, cause the processor 808 to execute any of the methods described herein.

The controller 800 may be physically or wirelessly coupled to one or more of the other components of the electroporation system 300 and may be configured to control operation of the electroporation system 300 via one or more user-initiated or automatic commands or parameters. The controller 800 may include the processor 808 (e.g., microcontroller/microprocessor, a central processing unit (CPU), a field-programmable gate array (FPGA) device, an application-specific integrated circuit (ASIC), another hardware device, a firmware device, or any combination thereof) and a non-transitory computer readable medium (such as memory) 804 configured to (and that does) store instructions, one or more data sets, or the like. A non-transitory computer readable medium may include any tangible or non-transitory storage media or memory media such as electronic, magnetic, or optical media. The terms “tangible” and “non-transitory,” as used herein, are intended to describe a non-transitory computer readable medium (such as memory) excluding propagating electromagnetic signals, but are not intended to otherwise limit the type of physical computer-readable storage device that is encompassed by the phrase non-transitory computer-readable medium or memory. For instance, the terms “non-transitory computer-readable medium” or “tangible memory” are intended to encompass types of storage devices that do not necessarily store information permanently, including for example, random access memory (RAM). Program instructions and data stored on a tangible computer-accessible storage medium in non-transitory form may further be transmitted by transmission media or signals such as electrical, electromagnetic, or digital signals, which may be conveyed via a communication medium such as a network and/or a wireless link.

The instructions of the memory may be executable by the processor 808 to perform or initiate one or more operations or functions as described herein. In some aspects, the controller 800 may include one or more interface(s), one or more I/O device(s), a power source, one or more sensor(s), signal generator (e.g., RF generator), or a combination thereof. For example, the controller 800 may include an I/O device that allows a user to input information (e.g., desired protocol) to control the operation of the electroporation system 300.

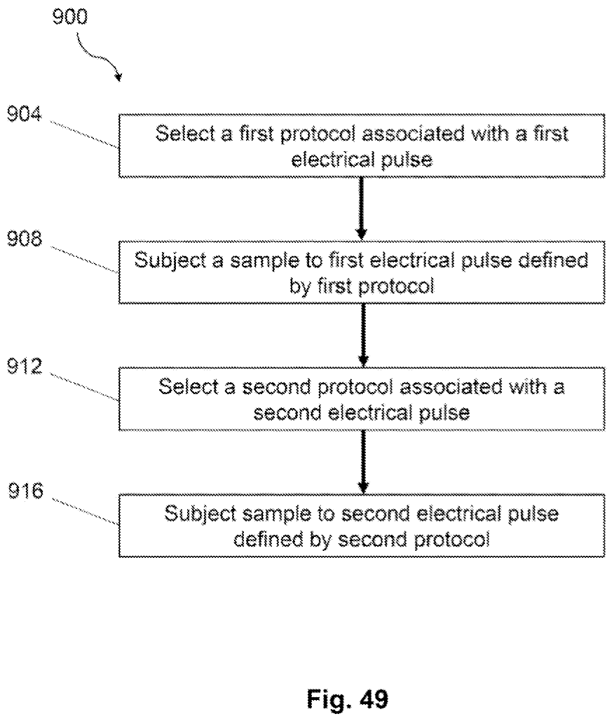

Referring now to FIG. 49, shown is an aspect 900 of the present methods for subjecting a sample to two or more electrical pulses, which may be implemented using electroporation system 300 depicted in FIG. 48. In the aspect shown, at step 904, non-transitory computer readable medium 804 of electroporation system 300 comprises instructions that, when executed by processor 808, cause the processor 808 to select a first protocol associated with a first electrical pulse having a first field strength and a first pulse duration. At step 908, in this aspect, controller 800 controls electroporation system 300 to create electric current of a first electrical pulse defined by the first protocol and send it through a sample comprising one or more intact cells, cell particles, or lipid vesicles, the first electrical pulse being sufficient to load the cells, cell particles, or lipid vesicles with an agent according to the first protocol. Optionally, after step 908, the sample is allowed to recover in culture at least, at most, or about 6, 12, 18, 24, 30, 36, 42, 48, 54, 60, 66, 72, 78, 84, 90, 96, 102, 108, 114, or 120 hours, or any range or value derivable therein. In some aspects, after step 908, the sample is allowed to recover in culture at most or at least 6 hours to 120 hours, 6 hours to 96 hours, 6 hours to 72 hours, 6 hours to 48 hours, 6 hours to 24 hours, 6 hours to 12 hours, or any range or value derivable therein. At step 912, in this aspect, the non-transitory computer readable medium 804 of electroporation system 300 comprises instructions that, when executed by processor 808, cause the processor 808 to select a second protocol associated with a second electrical pulse having a second field strength and a second pulse duration. At step 916, in this aspect, controller 800 controls electroporation system 300 to create electric current of a second electrical pulse defined by the second protocol and send it through the sample comprising one or more intact cells, cell particles, or lipid vesicles. The first field strength and/or the first pulse duration are different from the second field strength and/or second pulse duration, and the sample comprising one or more intact cells, cell particles, or lipid vesicles is subjected to the second electrical pulse, which is sufficient to load the cells, cell particles, or lipid vesicles with an agent according to the second protocol.

In some aspects, the electroporation system 300 may be controlled by the controller 800 to create electric current and send it through a cell solution. In some aspects, the current methods use a static electroporation apparatus. In some aspects, the current methods use a flow electroporation apparatus, which may be controlled by the controller 800 to create electrical current for electrical stimulation of suspensions of cells, cell particles, lipid vesicles, liposomes, tissues, or derivatives thereof, the flow electroporation apparatus having one or more inlet flow portals, one or more outlet flow portals, and one or more flow channels, the flow channels being comprised of two or more walls, with the flow channels further being configured to receive and transiently contain a continuous flow of particles in suspension from the inlet flow portals; and paired electrodes disposed in relation to the flow channels such that each electrode forms at least one wall of the flow channels, the electrodes further comprising placing the electrodes in electrical communication with a source of electrical energy, whereby suspensions flowing through the channels may be subjected to an electrical field formed between the electrodes.

In some aspects, flow electroporation is performed using MaxCyte STX®, MaxCyte VLX®, or MaxCyte GT® flow electroporation instrumentation. In some aspects, flow electroporation is performed using MaxCyte ExPERT STx®, MaxCyte ExPERT ATx®, MaxCyte ExPERT GTx®, or MaxCyte ExPERT VLx™. In specific aspects, static or flow electroporation is used with parameters described throughout the disclosure.

In some aspects, use of flow electroporation can help to overcome practical limitations with respect to the number of cells that can be electroporated, the time in which they can be electroporated, and the volume of solution in which they are suspended that attend to static or batch electroporation methods. With this method, a cell suspension is passed across parallel bar electrodes that are contained in a flow cell that may be disposable. It is to be understood that different configurations of flow cells can be used in the present disclosure. During this passage, the cells are subjected to electrical pulses with predetermined characteristics. The molecule of interest then diffuses into the cell following concentration and/or electrical gradients. Additionally, a population of lymphocytes can be transfected by electroporating the sample in less than 5 hours, preferably less than 4 hours, more preferably in less than 3 hours, and most preferably in less than 2 hours. The time of electroporation is the time that the sample is processed by the flow electroporation process. In certain aspects, 1E10 cells are transfected in 30 minutes or less using flow electroporation. In further aspects, 2E11 cells may be transfected in 30 minutes, or 60 minutes or less, using flow electroporation.

The flow electroporation process can be initiated by, for example, placing an electroporation chamber in fluid communication with solutions and cell suspensions in containers (e.g., via tubing), which may be carried out in an aseptic or sterile environment. A cell suspension and/or other reagents may be introduced to the electroporation chamber using one or more pumps, vacuums, valves, other mechanical devices that change the air pressure or volume inside the electroporation chamber and combinations thereof, which can cause the cell suspension and/or other reagents to flow into the electroporation chamber at a desired time and at the desired rate. If a portion of the cell suspension and/or other reagents is positioned in the electroporation chamber, electric pulses of a desired voltage, duration, and/or interval are applied to the cell suspension and/or other reagents. After electroporation, the processed cell suspension and/or other reagents can be removed from the electroporation chamber using one or more pumps, vacuums, valves, other electrical, mechanical, pneumatic, or microfluidic devices that change the displacement, pressure or volume inside the electroporation chamber, and combinations thereof. In certain aspects, gravity or manual transfer may be used to move sample or processed sample into or out of an electroporation chamber. If desired, a new cell suspension and/or other reagents can be introduced into the electroporation chamber. An electroporated sample can be collected separately from a sample that has not yet been electroporated. The preceding series of events can be coordinated temporally by a computer coupled to, for example, electronic circuitry (e.g., that provides the electrical pulse), pumps, vacuums, valves, combinations thereof, and other components that effect and control the flow of a sample into and out of the electroporation chamber. As an example, the electroporation process can be implemented by a computer, including by an operator through a graphic user interface on a screen (e.g., a monitor) and/or a keyboard. Examples of suitable valves include pinch valves, butterfly valves, and/or ball valves. Examples of suitable pumps include centrifugal or positive displacement pumps.

As an example, a flow electroporation device can comprise at least two electrodes separated by a spacer, where the spacer and the at least two electrodes define a chamber. In some aspects, the electroporation chamber can further comprise at least three ports traversing the spacer, where a first port is for sample flow into the chamber, a second port is for processed sample flow out of the chamber, and a third port is for non-sample fluid flow into or out of the chamber. In some aspects, the non-sample fluid flows out of the chamber when a sample flows into the chamber, and the non-sample fluid flows into the chamber when processed sample flows out of the chamber. As another example, a flow electroporation device can comprise an electroporation chamber having a top and bottom portion comprising at least two parallel electrodes, the chamber being formed between the two electrodes and having two chamber ports in the bottom portion of the electroporation chamber and two chamber ports in the top portion of the electroporation chamber. Such a device can further comprise at least one sample container in fluid communication with the electroporation chamber through a first chamber port in the bottom portion of the chamber, and the electroporation chamber can be in fluid communication with the sample container through a second chamber port in the top portion of the chamber, forming a first fluid path. Further, at least one product container can be in fluid communication with the electroporation chamber through a third chamber port in the bottom portion of the chamber, and the electroporation chamber can be in fluid communication with the product container through a fourth chamber port in the top portion of the chamber, forming a second fluid path. In some aspects, a single port electroporation chamber may be used. In other aspects, various other suitable combinations of electrodes, spacers, ports, and containers can be used. The electroporation chamber can comprise an internal volume of about 1-10 mL; however, in other aspects, the electroporation chamber can comprise a lesser internal volume (e.g., 0.75 mL, 0.5 mL, 0.25 mL, or less) or a greater internal volume (e.g., 15 mL, 20 mL, 25 mL, or greater). In some aspects, the electroporation chamber and associated components can be disposable (e.g., Medical Grade Class VI materials), such as PVC bags, PVC tubing, connectors, silicone pump tubing, and the like.

Any number of containers (e.g., 1, 2, 3, 4, 5, 6, or more) can be in fluid communication with the electroporation chamber. The containers may be collapsible, expandable, or fixed volume containers. For example, a first container (e.g., a sample source or sample container) can comprise a cell suspension and may or may not include a substance that will pass into cells in the cell suspension during electroporation. If the substance is not included, a second container comprising this substance can be included such that the substance can be mixed inline before entry into the electroporation chamber or in the electroporation chamber. In an additional configuration, another container may be attached, which can hold fluid that will be discarded. One or more additional containers can be used as the processed sample or product container. The processed sample or product container will hold cells or other products produced from the electroporation process. Further, one or more additional containers can comprise various non-sample fluids or gases that can be used to separate the sample into discrete volumes or unit volumes. The non-sample fluid or gas container can be in fluid communication with the electroporation chamber through a third and/or fourth port. The non-sample fluid or gas container may be incorporated into the processed sample container or the sample container (e.g., the non-sample fluid container can comprise a portion of the processed sample container or the sample container); and thus, the non-sample fluid or gas can be transferred from the processed sample container to another container (which may include the sample container) during the processing of the sample. The non-sample fluid or gas container may be incorporated into the chamber, as long as the compression of the non-sample fluid or gas does not affect electroporation. Further aspects of the disclosure may include other containers that are coupled to the sample container and may supply reagents or other samples to the chamber.

A flow electroporation apparatus that can be used in conjunction with the present disclosure is, in one aspect, comprised of the following: an electroporation system having a computer that communicates with an electronics module to run electroporation processes in real time and manage electroporation process-associated data and a monitor (e.g., which may be part of a mobile device or a device designed for use on a desk, table, cart, or the like) that displays a graphical user interface and enables user interaction. An operator inputs a desired voltage and other parameters into the flow electroporation system. As noted above, a range of settings is optionally available. The computer communicates with the electronics module to charge a capacitor bank to the desired voltage. Appropriate switches then manipulate the voltage before it is delivered to the flow path to create the electric field. The switches provide alternating pulses or bursts to minimize electrode wear brought on by prolonged exposure to the electric field. The voltage is delivered according to the duration and frequency parameters set into the flow electroporation system by the operator. Details of an example of a flow electroporation system is described in U.S. Pat. No. 7,186,559, which is incorporated herein by reference in its entirety.

The present electroporation systems and methods may also include processing assemblies, trays, gaskets, docking stations, racks, and vessels for delivery to the electroporation system.

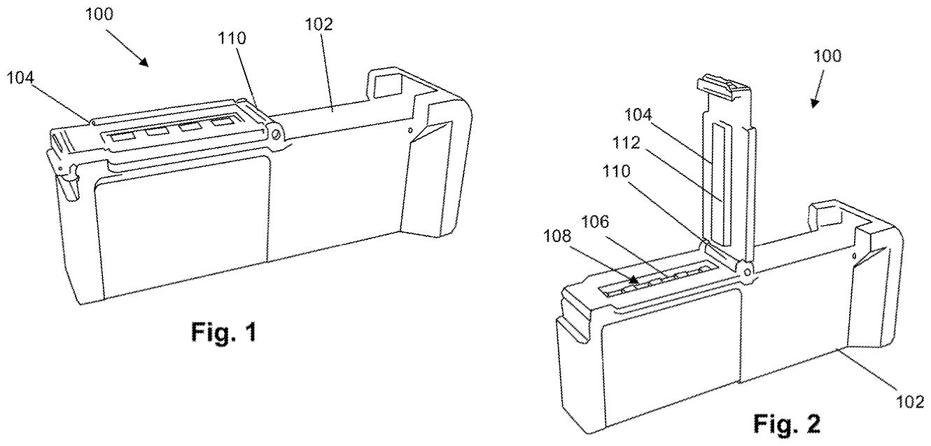

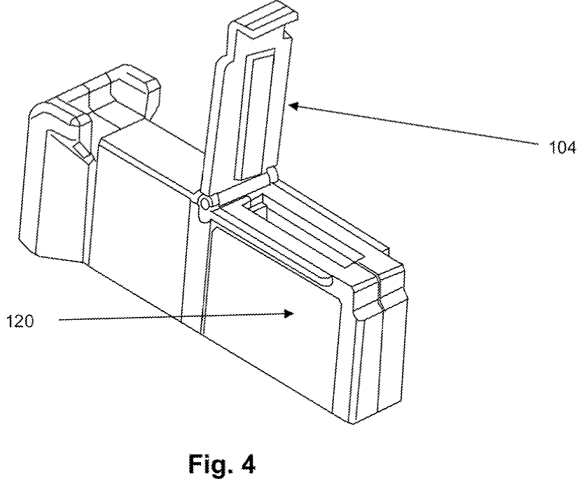

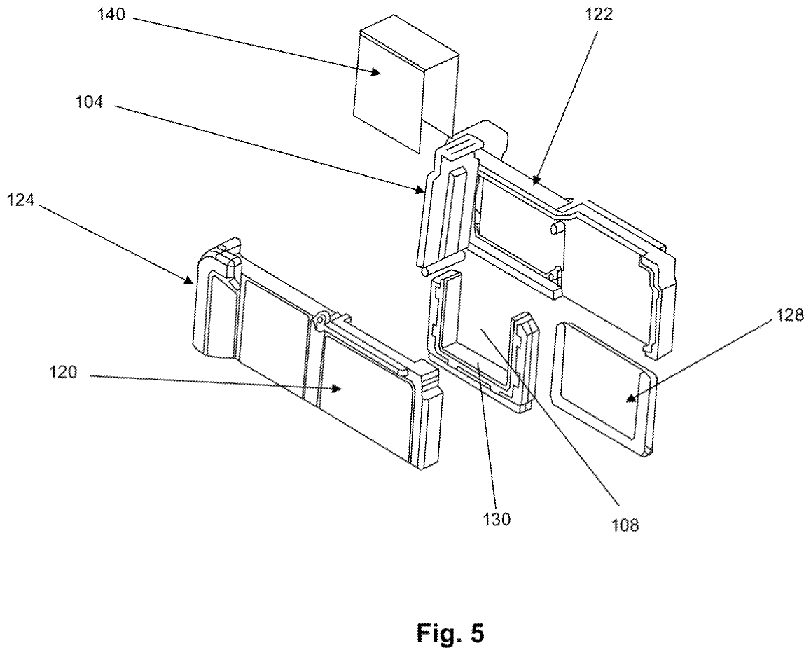

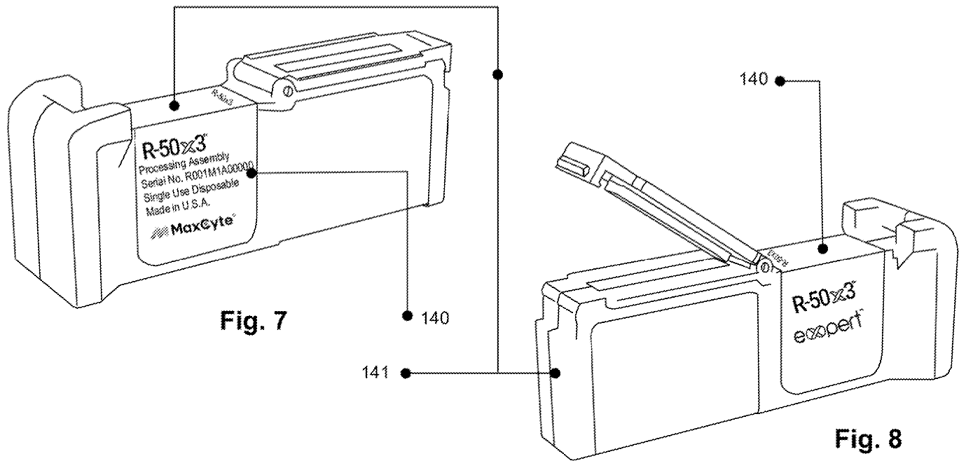

FIGS. 1-10 illustrate a processing assembly 100 consistent with aspects of this disclosure. The processing assembly 100 may be provided for use in electroporation systems and devices. The processing assembly 100 may include a housing 102 and a lid 104 that covers an opening 106 to a chamber 108. In some aspects, chamber 108 may receive samples, cultures, liquid media, etc. that may be provided to an electroporation system or device that processing assembly 100 may be compatible with.

Lid 104 may have a hinged connection 110 to the housing 102 that allows lid 104 to move between a closed position (FIG. 1) where the lid covers opening 106 and connects to housing 102 and an open position (FIG. 2) where the lid is hinged away from opening 106 and allows opening 106 to be exposed. The hinged connection 110 of lid 104 may provide improved handling and ease-of-use of processing assembly 100. In the closed position, lid 104 may prevent contamination of processing assembly 100. In some aspects, lid 104 may swivel about hinged connection 110 up to 180° and may connect to housing 102. In some aspects, lid 104 may connect to housing 102 via an interference fit where lid 104 clips to the housing 102. For example, the interference fit may connect lid 104 to housing 102 in the closed position at connection 109 and in an open position at connection 111. The interference fit may maintain a tight seal across wells within chamber 108 when lid 104 is closed. Lid 104 may further include a contoured surface 112 that may connect to and cover opening 106 and maintain an uncontaminated seal.

Processing assembly 100 may further include aluminum electrode buses 120 positioned around chamber 108 and may surround a gasket (e.g., gasket 130) within chamber 108. Housing 102 may include a left handle 122 and a right handle 124 that connect to each other to form housing 102. The left handle 122 and right handle 124 may be spaced apart by pins 125 that may be positioned opposite each other and may connect the left handle 122 and right handle 124. In some aspects, electrode buses 120 may be wrapped around right handle 124. In other aspects, electrode buses 120 may be positioned on one side of chamber 108 across from a gold-coated plastic film 128.

Processing assembly 100 may further include gold-coated plastic film 128 that may be received between the left handle 122 and right handle 124 and positioned opposite to the electrode buses 120 and framing gasket 130. Gold-coated plastic film 128 may have gold vacuum deposited on large rolls of plastic film that can be die cut to size and installed on processing assembly 100. In some aspects, the gold-coated film 128, the aluminum electrode bus 120, and adhesive layer rolls may be joined.

Processing assembly 100 may include a gasket 130 and plastic spacer that may be received in chamber 108. The gasket 130 may take at least one of several shapes and sizes as described in more detail below. For example, gasket 130 may be sized to receive samples of a variety of sizes including samples sized at 1000 μL, 400 μL, 100 μL, 100 μL×2, 50 μL×3, and 25 μL×3 variants, among others. In some aspects, gasket 130 may be made of silicone rubber or other flexible materials. Processing assembly 100 may be configured for use with any one of the gasket sizes and arrangements described herein such that processing assembly 100 may be used for any number of sized gaskets 130.

Processing assembly 100 may further include a device label 140 that extends around housing 102 away from electrode buses 120. In some aspects, device labels 140 may include a unique product serial number, size, instructions, logos, etc. Some aspects may also provide for writing space 141 on an end of processing assembly 100.

Processing assembly may provide several advantages, including an increased volume range of samples within chamber 108 and gasket 130, improved ease of use, and improvements in cell recovery and consistent performance. In some aspects, gold-coated plastic film 128 may provide a manufacturing cost reduction and may allow for reaction volumes of 25-1000 μL, e.g., 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 μL, or any range or value derivable therein, using a variety of gaskets.

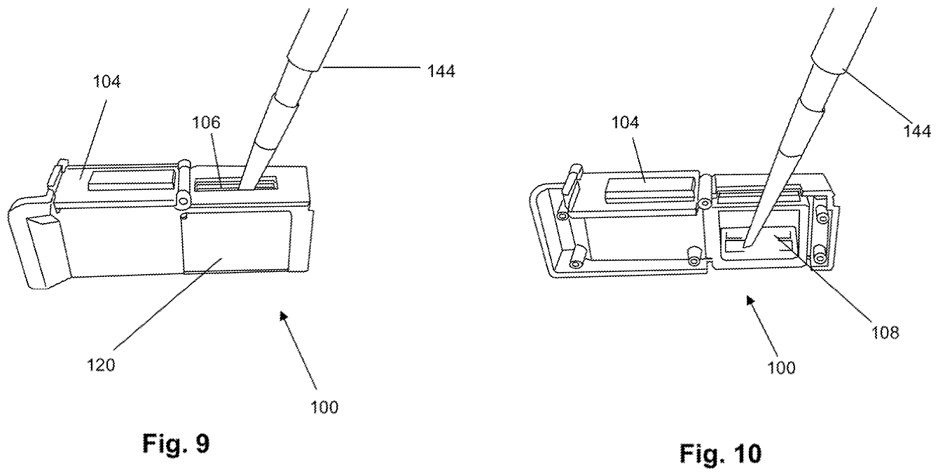

FIGS. 9 and 10 show processing assembly 100 may be configured to be filled via a loading device 144 that may be inserted into chamber 108 via opening 106 with lid 104 in the open position. Loading device 144 may fill chamber 108 with a sample for testing or treatment in an electroporation system. After loading device 144 provides the sample to chamber 108, loading device 144 may be removed and lid 104 may be closed to prevent contamination of the sample.

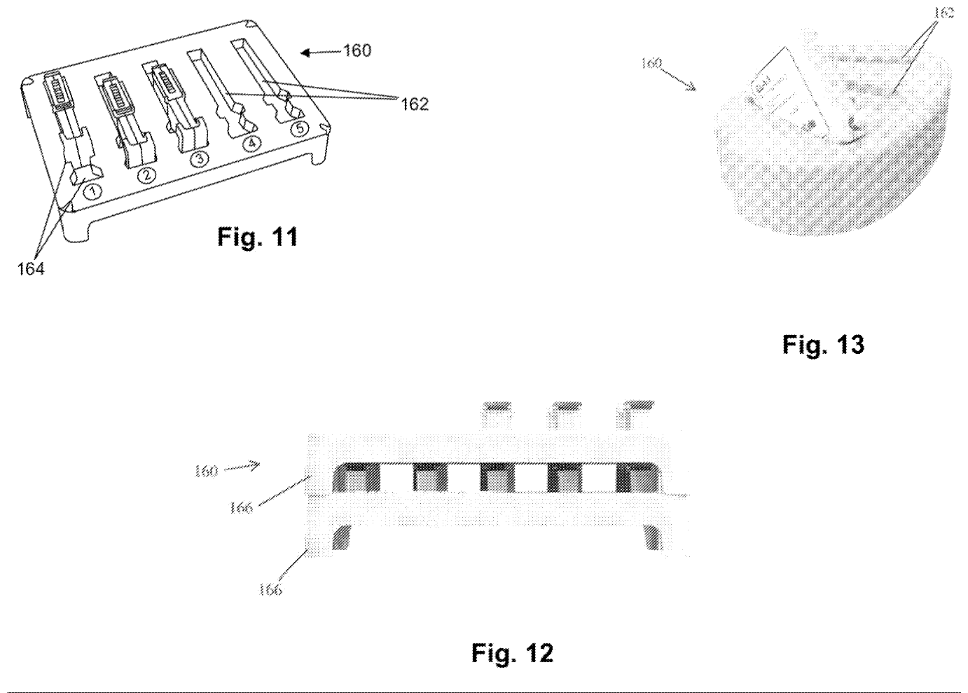

FIGS. 11-13 illustrate aspects of the present disclosure that may also provide one or more trays 160. Trays 160 may receive one or more processing assemblies (e.g., processing assembly 100 or other processing assemblies) in slots 162 spaced apart across the tray 106. In some aspects, trays 160 may be rectangular in shape and each slot 160 may be arranged parallel to the other slots 160. In other aspects, tray 160 may be curved, circular, or semi-circular and may have slots 160 arranged in a radial pattern around tray 160.

Tray 160 may include one or more positions for receiving processing assemblies. In some aspects, the tray may include one or more positions 164 such that the first position and second position may allow a user to distinguish a state (e.g., complete vs. incomplete, tested vs. untested, distinguish between sample type) of the processing assembly placed in tray 160. Trays 160 may have legs 166 that may allow one or more trays 160 to be stacked on top of each other while providing clearance for the processing assemblies loaded into the tray. Trays 160 may provide for improvements in the transportability and organization of processing assemblies and may allow for sterilization of an array of processing assemblies at once.

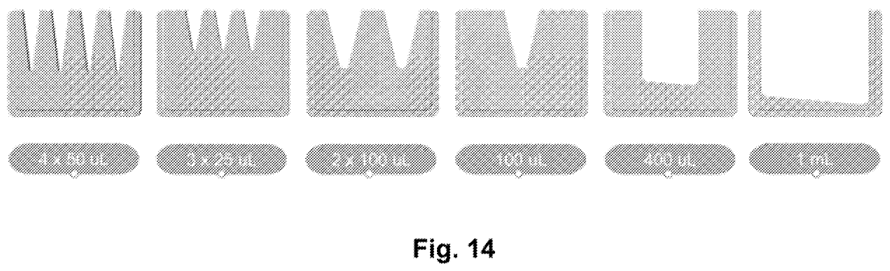

FIG. 14 illustrates a plurality of gaskets that could be implemented as gasket 130 within processing assembly 100 described above. Gasket 130 may be sized to receive samples of a variety of sizes, including samples sized at 4×50 μL, 3×25 μL, 2×100 μL, 100 μL, 400 μL, and 1 mL, among others. In some aspects, the 400 μL and 1 mL sized gaskets may have a sloped bottom surface that may provide for improved loading and unloading of samples.

In some aspects, the gaskets may provide flexibility where single/multi-well selections are designed to optimize workflow. Gaskets may also provide scalability by seamlessly shifting between small and large scale on a single platform. Gaskets may also provide improved functionality where functional design prevents contamination of samples while providing ease of use.

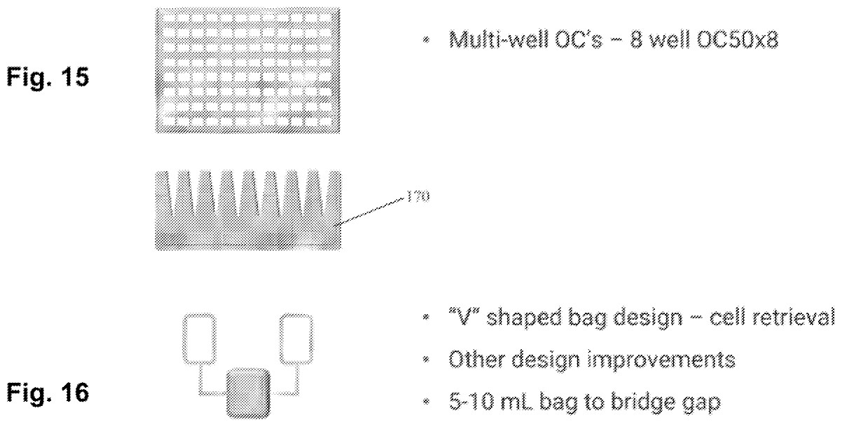

FIG. 15 illustrates a top view of an array of gaskets and a front view of a gasket, consistent with aspects of the present disclosure, where each gasket has eight wells.

FIG. 16 illustrates a front view of a bag and processing apparatus consistent with aspects of the present disclosure. The processing apparatus may have a V-shaped design for cell retrieval. Additionally, the processing assembly may include a 5-10 mL bag, e.g., a 5, 6, 7, 8, 9, or 10 mL, or any range or value derivable therein, bag, to bridge a gap.

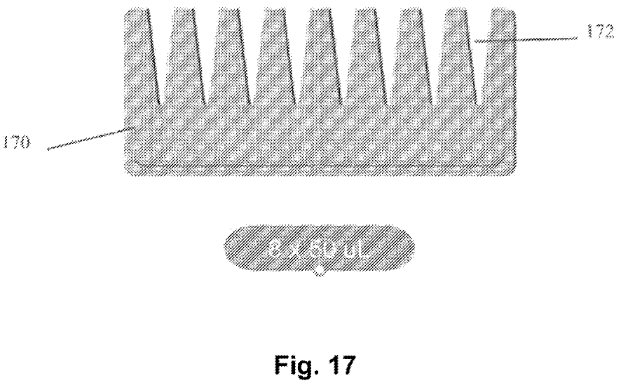

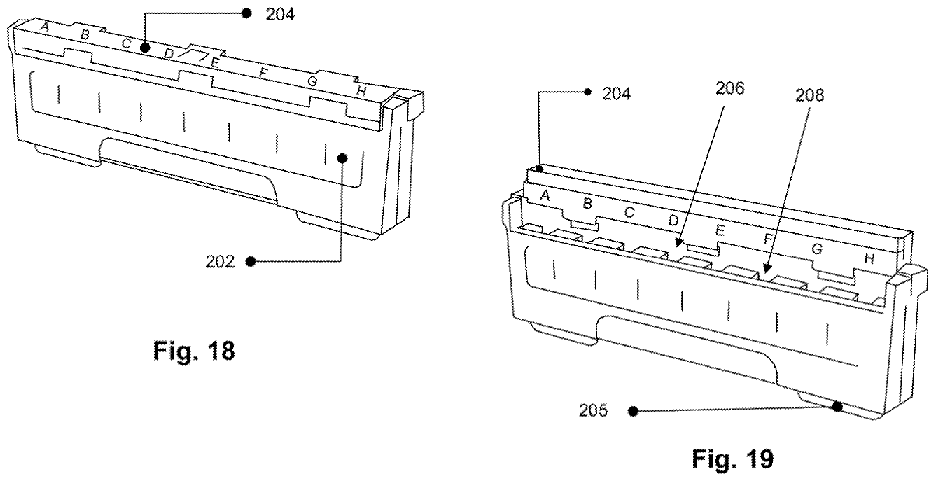

FIG. 17 illustrates a gasket 170 having eight wells 172, which may be sized for samples of 50 μL in each well 172. Gasket 170 may be configured to be received or inserted into a multi-well processing assembly 200. FIGS. 18-20 illustrate multi-well processing assembly 200 that may be configured to allow processing of multiple loaded wells (e.g., wells 172) by an electroporation system.

Multi-well processing assembly 200 may include a housing 202 with a lid 204 that extends along the length of the housing and covers an opening 206 to a chamber 208. In some aspects, chamber 108 may receive samples, cultures, liquid media, etc., that may be provided to an electroporation system or device that processing assembly 200 may be compatible with.

Lid 204 may have a hinged connection 210 to one side of the housing 202 that allows lid 204 to move between a closed position (FIG. 18) where the lid covers opening 206 and connects to housing 202 and an open position (FIG. 19) where the lid is hinged away from opening 206 and allows opening 206 to be exposed. In the closed position, lid 204 may prevent contamination of processing assembly 200. In some aspects, lid 204 may connect to housing 202 via an interference fit where lid 204 clips to the housing 202. In some aspects, lid 204 may be removable from the housing 202. In some aspects, processing assembly 200 may have a base 205 that allows the housing 202 to stand on its own, which may provide for ease of use, loading, and stability during loading.

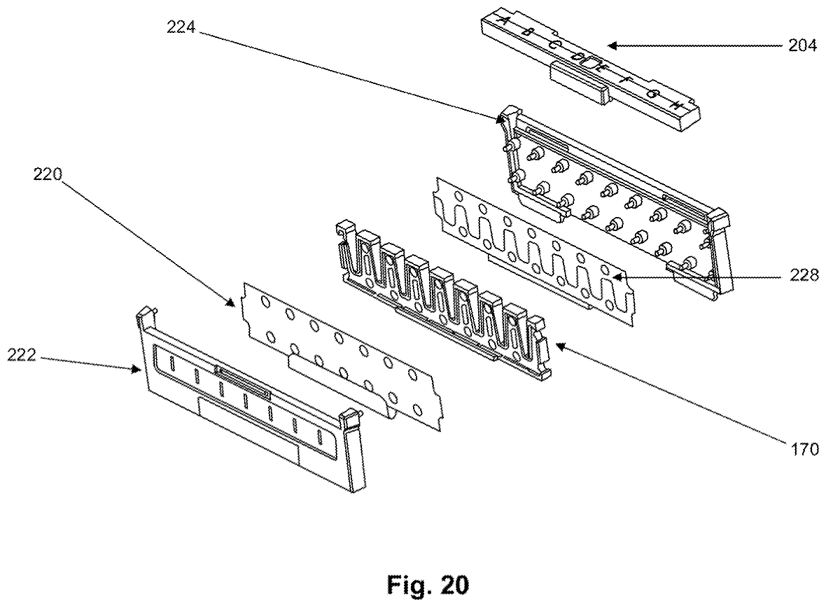

Processing assembly 200 may further include aluminum electrode buses 220 positioned around chamber 208 and that may surround a gasket (e.g., gasket 170) within chamber 208. Housing 202 may include a left handle 222 and a right handle 224 that connect to each other to form housing 202 (e.g., FIG. 20). The left handle 222 and right handle 224 may be spaced apart by pins 225 that may be positioned opposite each other and may connect the left handle 222 and right handle 224. In some aspects, electrode buses 220 may be wrapped around right handle 224. In other aspects, electrode buses 220 may be positioned on one side of chamber 208 across from a gold-coated plastic film 228.

Processing assembly 200 may further include a gold-coated plastic film 228 that may be received between the left handle 122 and right handle 124 and positioned opposite to the electrode buses 120 and framing gasket 130. Gold-coated plastic film 128 may have gold vacuum deposited on large rolls of plastic film that can be die cut to size and to be installed on processing assembly 100. In some aspects, the gold-coated film 128, the aluminum electrode bus 120, and adhesive layer rolls may be joined. In some aspects, gold-coated plastic film 228 may have gold coating arranged in a shape that mirrors or follows the shape of gasket 170.

Processing assembly 200 may include a gasket 170 and plastic spacer that may be received in chamber 208. The gasket 170 may take at least one of several shapes. For example, gasket 170 may have eight wells 172, which may be sized for samples of 50 μL in each well 172. In some aspects, gasket 170 may be made of silicone rubber or other flexible materials. Processing assembly 200 may be configured for use with any gasket size and arrangements described herein such that the processing assembly 200 may be used for any number of sized gaskets 170.

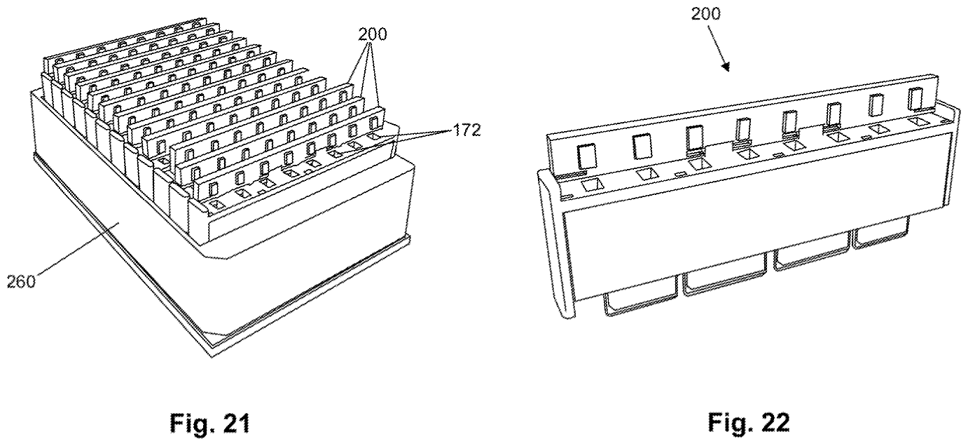

FIG. 21 illustrates a tray 260 configured to receive a plurality of multi-well processing assemblies 200. As illustrated in FIGS. 21 and 22, multi-well processing assemblies may be loaded into tray 260 without lids. Tray 260 may receive twelve processing assemblies 200, and each processing assembly may include eight wells (e.g., wells 172). Accordingly, each tray 260 may include ninety-six wells.

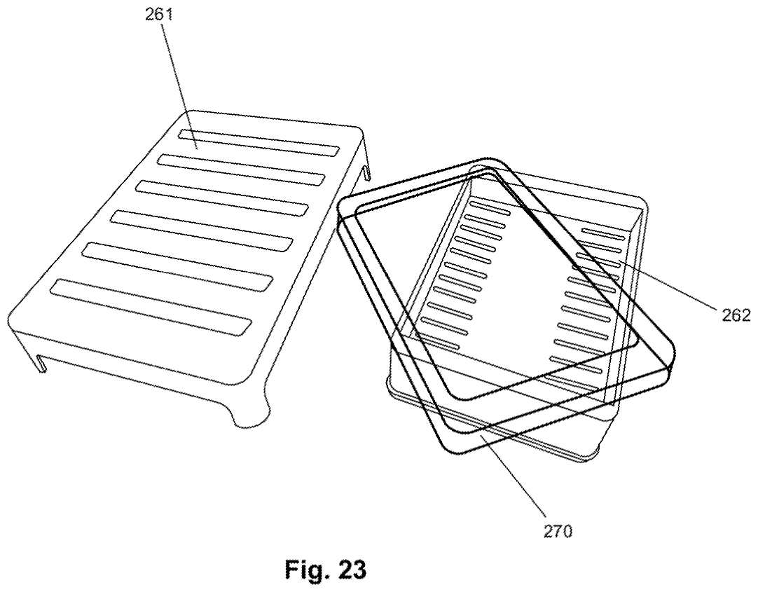

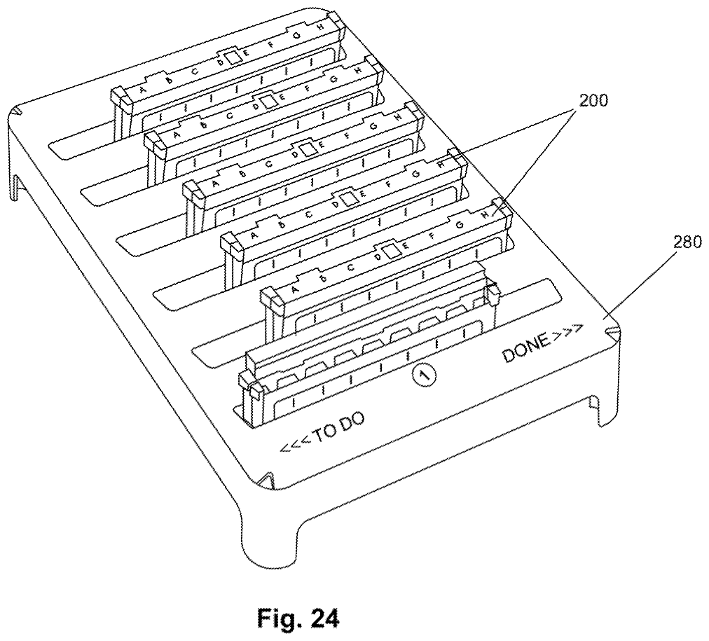

FIG. 23 illustrates a tray 261 configured to receive six processing assemblies 200, which may be used in a manual workflow, and a tray 262 configured to receive twelve processing assemblies or twelve individual gasket samples, which may include a cover or lid closure 270.

FIG. 24 illustrates a multi-well rack 280 that can receive a plurality of processing assemblies 200 and may provide for loading, unloading, and organization of processing assemblies 200.

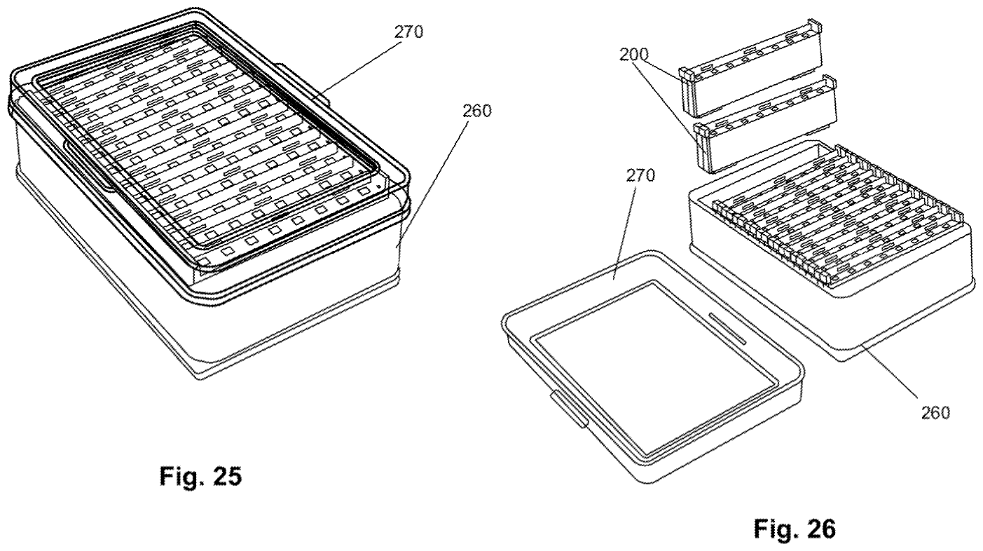

FIGS. 25 and 26 illustrate tray 260 with a cover or lid closure 270 and the loading and unloading of processing assemblies 200 into tray 260.

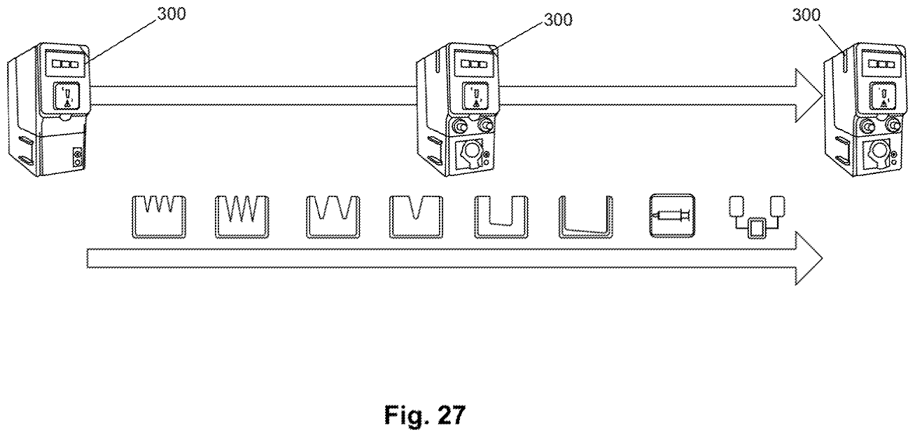

FIG. 27 illustrates exemplary electroporation systems 300 with which the disclosed aspects may be compatible.

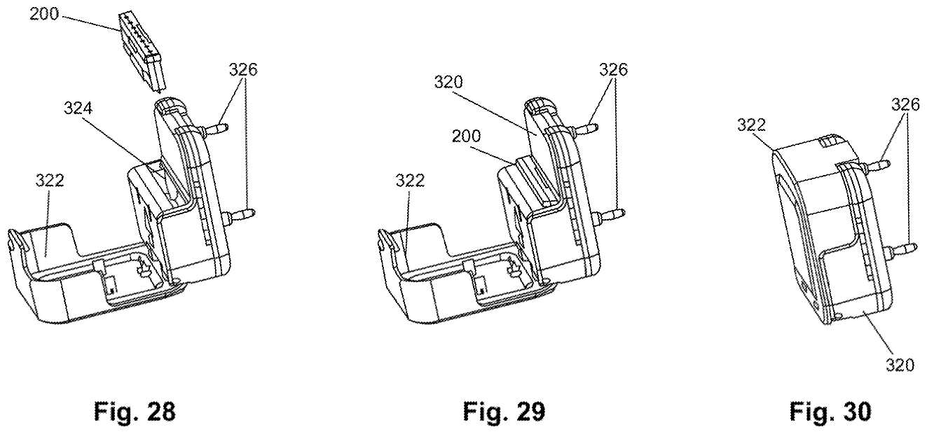

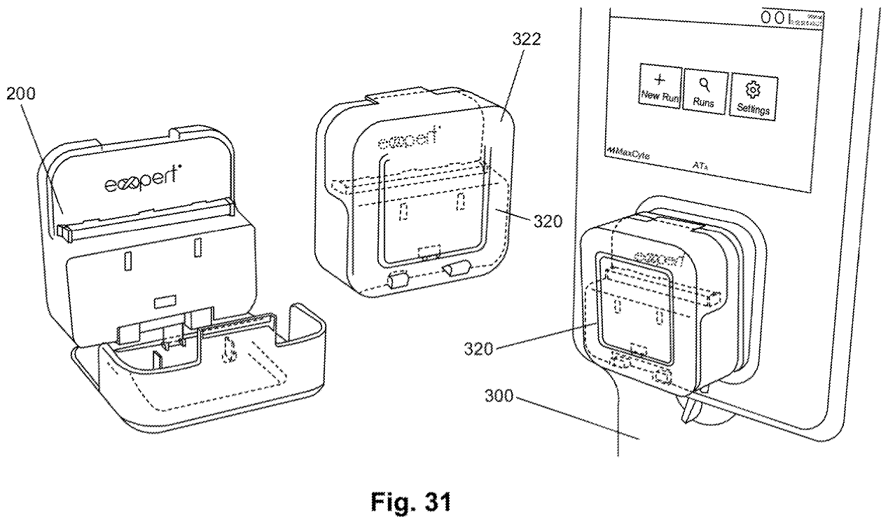

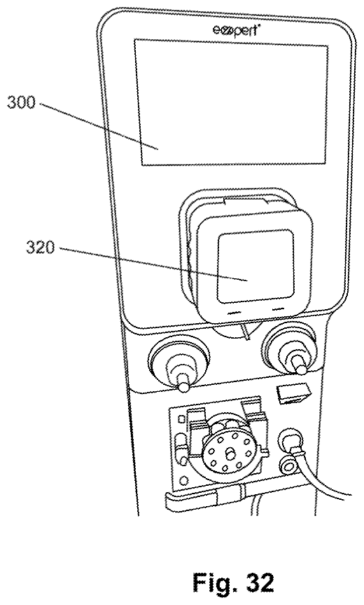

FIGS. 28-32 illustrate a docking station 320 that may connect processing assemblies (e.g., processing assembly 200) to an electroporation system (e.g., electroporation system 300). Docking station 320 may include a lid 322 that may be connected via a hinge connection to docking station 320. Lid 322 may be configured to move between an open position (FIGS. 28 and 29) and a closed position (FIG. 30). Docking station 320 may have a port 324 configured to receive one or more processing assemblies 200. Docking station 320 may also have electrical contacts 326 that may connect to receptacles on an electroporation system (e.g., electroporation system 300).

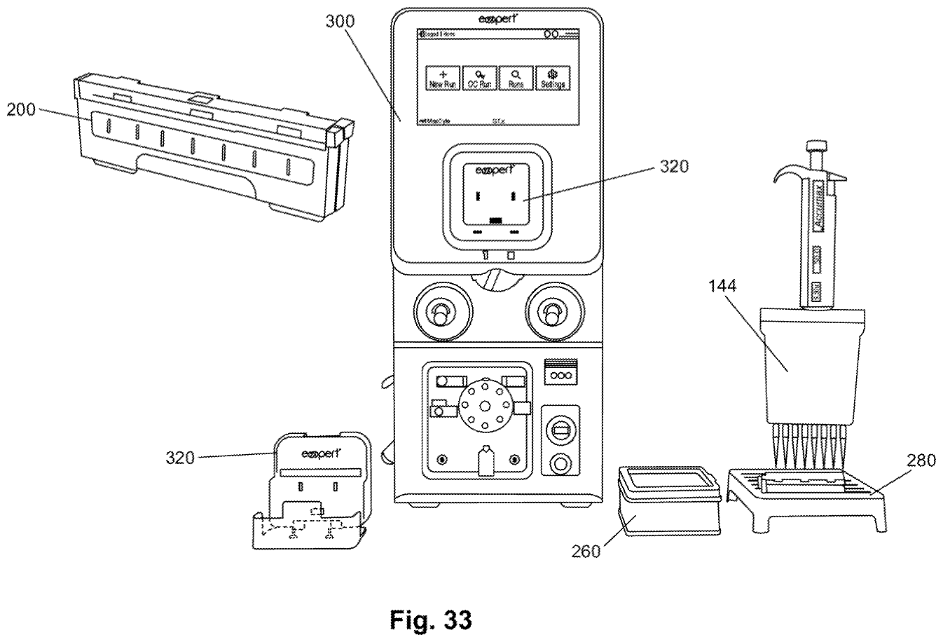

FIG. 33 shows the multi-well processing assembly 200, electroporation system 300, docking station 320, tray 260, loading device 144, and rack 280.

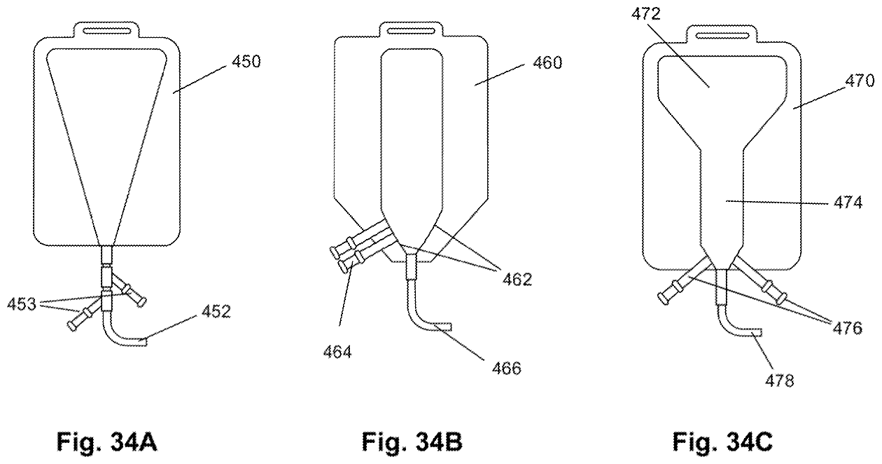

FIGS. 34A-34C illustrate exemplary aspects of bags for use in flow electroporation assemblies. As shown in FIG. 34A, bag 450 may include a V-shape interior that drains into outlet 452 that may have a plurality of connectors 453. As shown in FIG. 34B, bag 460 may include a narrower inner chamber having angled lower surfaces 462, one of the lower surfaces 462 may include one or more connectors 464 and the bag 460 may also include a centrally positioned outlet 466. As shown in FIG. 34C, bag 470 may include a wide upper chamber 472 and a narrow lower chamber 474, the lower chamber 474 may include connectors 476 at each angled bottom surface and a centrally positioned outlet 478. Bags 450, 460, 470 may include Luer fittings, Luer-activated ports, tubing, tube clamps and labels (see diagram). Bags may be used as a sample bag, a collection bag, and an air bag.

### III. Electroporation Targets

Targets for electroporation include a number of cell types or particles derived from a number of organisms and sources. In some aspects, the target can be nucleated or anucleated cells or particles. Cells or particles of the disclosure can be primary cells or a cell line or a particle derived therefrom. For example, a target may be prokaryotic, yeast, insect, mammalian, rodent, hamster, primate, human, bird, plant cells, or portions/fragments thereof. In certain aspects, the present disclosure relates to compositions, methods, and apparatuses for the introduction of agents of interest into various types of living cells or cell particles or synthetic vesicles or liposomes. More particularly, the present disclosure relates to a method and apparatus for the introduction of agents of interest into cells, cell particles, lipid vesicles, liposomes, tissues, or derivatives thereof. These electroporation targets can be utilized as an agent delivery system to target a site of infection, metastasis, or other pathologic lesion.

A. Cell Culture

As used herein, the terms “cell,” “cell line,” and “cell culture” may be used interchangeably. All of these terms also include both freshly isolated cells and ex vivo cultured, activated, or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain aspects, electroporation can be carried out on any prokaryotic or eukaryotic cell. In some aspects, electroporation involves electroporation of a mammalian cell. In some aspects, the mammalian cell is a human cell. In other aspects, the mammalian cells is an animal cell, for example, a murine cell, rat cell, hamster cell, or primate cell.

In certain aspects, electroporation involves electroporation of a cell line or a hybrid cell type. In some aspects, the cell or cells being electroporated are cancer cells, tumor cells, or immortalized cells. In some instances, tumor, cancer, immortalized cells, or cell lines are induced, and in other instances, tumor, cancer, immortalized cells or cell lines enter their respective state or condition naturally.

In certain aspects, the cells or cell lines electroporated can be 697, 10T½, 1321N1, A549, AHR77, B-cells, B-LCL, B16, B65, Ba/F3, BHK, C2C12, C6, CaCo-2, CAP/, CAP-T, CaSki, ChaGo-K-1, CHO, CHO2, CHO-DG44, CHO-K1, COS, COS-1, Cos-7, CV-1, Dendritic cells, DG75, DLD-1, EL4, Embryonic Stem (ES) Cells or derivatives, H1299, HaCaT, HAP1, HCT116, HEK, 293, 293T, 293FT, HeLa, Hep G2, HL60, Hematopoietic Stem Cells, HOS, HT1080, HT29, Huh-7, HUVEC, Induced Pluripotent Stem (iPS) Cell or derivative, INS-1/GRINCH, Jurkat, K46, K562, KG1, KHYG-1, L5278Y, L6, LNCaP, LS180, MCF7, MDA-MB-231, MDCK, ME-180, Mesenchymal Cells, MG-63, Min-6, Monocytic cell, MOLT4, Nalm6, ND7/23, Neuro2a, NK92, NIH 3T3, NIH3T3L1, NS/0, NK-cells, P3U1, Panc-1, PC12, PC-3, PER.C6, PM1, Peripheral blood cells, Plasma cells, Primary Fibroblasts, Ramos, RAW 264.7, RBL, Renca, RLE, SF21, SF9, SH-SY5Y, SK-BR-3, SK-MES-1, SK-N-SH, SK-OV-3, SP3/0, SL3, SW403, Stimulus-triggered Acquisition of Pluripotency (STAP) cell or derivate SW403, T-cells, THP-1, Tumor cells, U2OS, U205, U937, peripheral blood lymphocytes, expanded T cells, hematopoietic stem cells, YB2/0, Vero cells, or derivatives thereof.

In some aspects, the cells are adipocytes, chondrocytes, endothelial cells, epithelial cells, fibroblasts, hepatocytes, keratinocytes, myocytes, neurons, osteocytes, peripheral blood lymphocytes, peripheral blood mononuclear cells (PBMCs), expanded T cells, splenocytes, stem cells, hematopoietic stem cells, or thymocytes. In some aspects, the cells are primary cells. In some aspects, the cells are cultured cells. In some aspects, the cells are cultured cell lines. In some aspects, the PBMCs are peripheral blood lymphocytes (PBLs). In some aspects, the PBLs are natural killer (NK) cells, T cells, or B cells. In some aspects, the PBMCs are monocytes. In some aspects, the monocytes are macrophages or dendritic cells. In some aspects, the macrophages are microglia. In some aspects, the stem cells are adipose stem cells, embryonic stem cells, hematopoietic stem cells, induced pluripotent stem cells, mesenchymal stem cells, or neural stem cells. One or more of the cells disclosed above is excluded is some aspects.

In some aspects, the cells are primary cells isolated from a patient. In some aspects, the cells are freshly isolated. The isolated cells can be allogeneic cells and can be obtained from standard sources, for example, hospital services. Donors can be screened using histories and standard blood tests. In some aspects, the cells are transfected at a time period of less than or exactly 20, 19, 18, 17, 16, 15, 14, 13, 12, 11, 10, 9, 8, 7, 6, 5, 4, 3, 2, 1 days or any value from 20 to 1 days or any derivable range therein, or less than or exactly 24, 22, 20, 18, 16, 14, 12, 10, 8, 6, 4, 2, 1 hours or any value from 24 to 1 hours or any derivable range therein. In some aspects, the isolated cells have never been frozen. In some aspects, the isolated cells have never been passaged, or cultured, in vitro. In some aspects, the isolated cells have been passaged, or cultured, for less than or exactly 1, 2, 3, 4, 5, 6, 7, 8, 9, or 10 times, or any derivable range therein. The term “passaged” is intended to refer to the process of culturing and splitting cells in order to produce large number of cells from pre-existing ones. Passaging involves splitting the cells and transferring a small number into each new vessel for culturing. For adherent cultures, cells first need to be detached, commonly done with a mixture of trypsin-EDTA. A small number of detached cells can then be used to seed a new culture, while the rest is discarded. Also, the amount of cultured cells can easily be enlarged by distributing all cells to fresh flasks.

In certain aspects, the cell is one that is known in the art to be difficult to transfect. Such cells are known in the art and include, for example, primary cells, insect cells, SF9 cells, Jurkat cells, CHO cells, stem cells, slowly dividing cells, and non-dividing cells. In some aspects, the cell is a germ cell such as an egg cell or sperm cell. In some aspects, the cell is a fertilized embryo. In some aspects, the cell is a human fertilized embryo.

In some aspects, the cells maintain a high viability during and after the sequential electroporation process. Cell viability can be measured by methods known in the art. For example, cells can be counted before and after electroporation by a cell counter apparatus. In other aspects, apoptosis is measured. It is believed that introduction of large amounts of nucleic acids may induce apoptosis. It is contemplated that methods described herein will lead to less apoptosis than other methods in the art. In certain aspects, the amount of cells exhibiting apoptosis after sequential electroporation is less than 50, 45, 40, 35, 30, 25, 20, 15, 10, or 5%. Apoptosis refers to the specific process of programmed cell death and can be measured by methods known in the art. For example, apoptosis may be measured by Annexin V assays, activated caspase 3/7 detection assays, and Vybrant® Apoptosis Assay (Life Technologies).

Viability is routinely more than 50% or greater. Viability of sequentially electroporated cells can be at most or at least about 5%, 10%, 15%, 20%, 25%, 30%, 35%, 40%, 45%, 50%, 55%, 60%, 65%, 70%, 75%, 80%, 85%, 90% or 95% (or value from 5% to 95% or any range derivable therein), of the viability of the starting, unelectroporated population or an electroporated population transfected with a control construct. In some aspects, cell viability can be, can be at least, or can be at most 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% 12 to 96 hours after a second electrical pulse administered according to an electroporation method comprising subjecting a sample comprising one or more intact cells, cell particles, or lipid vesicles to a first electrical pulse having a first field strength and a first pulse duration sufficient to load the cells, cell particles, or lipid vesicles with an agent according to a first protocol; and subjecting the sample to a second electrical pulse having a second field strength and a second pulse duration sufficient to load the cells, cell particles, or lipid vesicles with an agent according to a second protocol. In some aspects, cell viability is at least 50% 12 to 96 hours after a second electrical pulse. In some aspects, cell viability is at least 60% 12 to 96 hours after a second electrical pulse. In some aspects, cell viability is at least 70% 12 to 96 hours after a second electrical pulse. In some aspects, cell viability is at least 80% 12 to 96 hours after a second electrical pulse. In some aspects, cell viability is at least 90% 12 to 96 hours after a second electrical pulse.

In some aspects, the electroporated cells are approximately 50% to 90% viable 12 to 96 hours after a second electrical pulse. In some aspects, the electroporated cells are approximately 50% to 90% viable 12 to 72 hours after a second electrical pulse. In some aspects, the electroporated cells are approximately 50% to 90% viable 12 to 48 hours after a second electrical pulse. In some aspects, the electroporated cells are approximately 50% to 90% viable 24 hours after a second electrical pulse. In some aspects, the electroporated cells are approximately 60% to 90% viable 12 to 96 hours after a second electrical pulse. In some aspects, the electroporated cells are approximately 60% to 90% viable 12 to 72 hours after a second electrical pulse. In some aspects, the electroporated cells are approximately 60% to 90% viable 12 to 48 hours after a second electrical pulse. In some aspects, the electroporated cells are approximately 60% to 90% viable 24 hours after a second electrical pulse.

In some aspects, cells may be subjected to limiting dilution methods to enable the expansion of clonal populations of cells. The methods of limiting dilution cloning are well known to those of skill in the art. Such methods have been described, for example, for hybridomas but can be applied to any cell. Such methods are described in “Cloning hybridoma cells by limiting dilution,” Journal of Tissue Culture Methods, 1985, Volume 9, Issue 3, pp 175-177, by Joan C. Rener, Bruce L. Brown, and Roland M. Nardone, which is incorporated by reference herein.

In some aspects, cells are cultured before electroporation or after electroporation. In some aspects, cells are allowed to recover in culture before electroporation or after electroporation. As used herein, “allowing a sample to recover,” “recovering a sample,” or “recover in culture” means culturing cells, including but not limited to cells of a sample, in any of the cell-culture vessels and cell culture media disclosed herein under conditions such as those disclosed herein that are appropriate and sufficient to facilitate restoration or return of the cells to an improved or desired state or condition. For example, recovery in culture may allow the cells to recover from the trauma of electroporation by, for instance, repairing cell walls, and to begin expressing or metabolizing an agent loaded into the cells upon electroporation of the cells.

In other aspects, cells are cultured during the selection phase after electroporation. In yet other aspects, cells are cultured during a maintenance and clonal selection and initial expansion phase. In still other aspects, cells are cultured during a screening phase. In other aspects, cells are cultured during a large scale production phase. Methods of culturing suspension and adherent cells are well-known to those skilled in the art.

In certain aspects, the density of cells during electroporation is a controlled variable. The cell density of cells during electroporation may vary or be varied according to, but not limited to, cell type, desired electroporation efficiency or desired viability of resultant electroporated cells. In certain aspects, the cell density is constant throughout electroporation. In other aspects, cell density is varied during the electroporation process. In certain aspects, cell density before electroporation may be in the range of 1×10⁴cells/mL to (y)×10⁴, where y can be 2, 3, 4, 5, 6, 7, 8, 9, or 10 (or any value from 2 to 10 or range derivable therein). In other aspects, the cell density before electroporation may be in the range of 1×10⁵cells/mL to (y)×10⁵, where y is 2, 3, 4, 5, 6, 7, 8, 9, or 10 (or any value from 2 to 10 or range derivable therein). In yet other aspects, the cell density before electroporation may be in the range of 1×10⁶ cells/mL to (y)×10⁶, where y can be 2, 3, 4, 5, 6, 7, 8, 9, or 10 (or any value from 2 to 10 or range derivable therein). In certain aspects, cell density before electroporation may be in the range of 1×10⁷cells/mL to (y)×10⁷, where y can be 2, 3, 4, 5, 6, 7, 8, 9, or 10 (or any value from 2 to 10 or range derivable therein). In yet other aspects, the cell density before electroporation may be in the range of 1×10⁷cells/mL to 1×10⁸ cells/mL, 1×10⁸ cells/mL to 1×10⁹ cells/mL, 1×10⁹ cells/mL to 1×10¹⁰ cells/mL, 1×10¹⁰ cells/mL to 1×10¹¹ cells/mL, 1×10¹¹ cells/mL to 1×10¹² cells/mL, or any value from 1×10⁷cells/mL to 1×10¹²cells/mL or range derivable therein. In certain aspects, the cell density before electroporation may be (y)×10⁶, where y can be any of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, or any value from 0.01 to 100 or range derivable therein. In certain aspects, the cell density before electroporation may be (y)×10¹⁰, where y can be any of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 (or any value from 0.01 to 1000 or range derivable therein).

In certain aspects the density of cells during electroporation is a controlled variable. The cell density of cells during electroporation may vary or be varied according to, but not limited to, cell type, desired electroporation efficiency or desired viability of resultant electroporated cells. In certain aspects, the cell density is constant throughout electroporation. In other aspects, cell density is varied during the electroporation process. In certain aspects, cell density during electroporation may be in the range of 1×10⁴cells/mL to (y)×10⁴, where y can be 2, 3, 4, 5, 6, 7, 8, 9, or 10 (or any value from 2 to 10 or range derivable therein). In other aspects, the cell density during electroporation may be in the range of 1×10⁵ cells/mL to (y)×10⁵, where y is 2, 3, 4, 5, 6, 7, 8, 9, or 10 (or any value from 2 to 10 or range derivable therein). In yet other aspects, the cell density during electroporation may be in the range of 1×10⁶cells/mL to (y)×10⁶, where y can be 2, 3, 4, 5, 6, 7, 8, 9, or 10 (or any value from 2 to 10 or range derivable therein). In certain aspects, cell density during electroporation may be in the range of 1×10⁷cells/mL to (y)×10⁷, where y can be 2, 3, 4, 5, 6, 7, 8, 9, or 10 (or any value from 2 to 10 or range derivable therein). In yet other aspects, the cell density during electroporation may be in the range of 1×10⁷cells/mL to 1×10⁸ cells/mL, 1×10⁸ cells/mL to 1×10⁹ cells/mL, 1×10⁹ cells/mL to 1×10¹⁰ cells/mL, 1×10¹⁰ cells/mL to 1×10¹¹ cells/mL, 1×10¹¹ cells/mL to 1×10¹² cells/mL, or any value from 1×10⁷cells/mL to 1×10¹²cells/mL or range derivable therein. In certain aspects, the cell density during electroporation may be (y)×10⁶, where y can be any of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, or any value from 0.01 to 100 or range derivable therein. In certain aspects, the cell density during electroporation may be (y)×10¹⁰, where y can be any of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 (or any value from 0.01 to 1000 or range derivable therein).

In certain aspects cell density after electroporation may be in the range of 1×10⁴cells/mL to (y)×10⁴, where y can be 2, 3, 4, 5, 6, 7, 8, 9, or 10 (or any value from 2 to 10 or range derivable therein). In other aspects, the cell density after electroporation may be in the range of 1×10⁵cells/mL to (y)×10⁵, where y is 2, 3, 4, 5, 6, 7, 8, 9, or 10 (or any value from 2 to 10 or range derivable therein). In yet other aspects, the cell density after electroporation may be in the range of 1×10⁶cells/mL to (y)×10⁶, where y can be 2, 3, 4, 5, 6, 7, 8, 9, or 10 (or any value from 2 to 10 or range derivable therein). In certain aspects, cell density after electroporation may be in the range of 1×10⁷cells/mL to (y)×10⁷, where y can be 2, 3, 4, 5, 6, 7, 8, 9, or 10 (or any value from 2 to 10 or range derivable therein). In yet other aspects, the cell density after electroporation may be in the range of 1×10⁷ cells/mL to 1×10⁸ cells/mL, 1×10⁸ cells/mL to 1×10⁹ cells/mL, 1×10⁹ cells/mL to 1×10¹⁰ cells/mL, 1×10¹⁰ cells/mL to 1×10¹¹ cells/mL, 1×10¹¹ cells/mL to 1×10¹² cells/mL, or any value from 1×10⁷ cells/mL to 1×10¹² cells/mL or range derivable therein. In certain aspects, the cell density after electroporation may be (y)×10⁶, where y can be any of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, or any value from 0.01 to 100 or range derivable therein. In certain aspects, the cell density after electroporation may be (y)×10¹⁰, where y can be any of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 (or any value from 0.01 to 1000 or range derivable therein).

In some instances, a certain number of cells can be electroporated in a certain amount of time. Given the flexibility, consistency and reproducibility of the described platform, up to or more than about (y)×10⁴, (y)×10⁵, (y)×10⁶, (y)×10⁷, (y)×10⁸, (y)×10⁹, (y)×10¹⁰, (y)×10¹¹, (y)×10¹², (y)×10¹³, (y)×10¹⁴, or (y)×10¹⁵ cells (or any value from (y)×10⁴ to (y)×10¹⁵ or range derivable therein) can be electroporated, where y can be any of 1, 2, 3, 4, 5, 6, 7, 8, or 9 (or value from 1 to 9 or range derivable therein), in less than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90 or 100 seconds (or any value from 0.01 seconds to 100 seconds or range derivable therein). In other instances, up to or more than about (y)×10⁴, (y)×10⁵, (y)×10⁶, (y)×10⁷, (y)×10⁸, (y)×10⁹, (y)×10¹⁰, (y)×10¹¹, (y)×10¹², (y)×10¹³, (y)×10¹⁴, or (y)×10¹⁵ cells (or any value from (y)×10⁴ cells to (y)×10¹⁵ cells or range derivable therein) can be electroporated, where y can be any of 1, 2, 3, 4, 5, 6, 7, 8, or 9 (or value from 1 to 9 or range derivable therein), in less than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100. 110, or 120 minutes (or any value from 0.01 minutes to 120 minutes or range derivable therein). In yet other aspects, up to or more than about (y)×10⁴, (y)×10⁵, (y)×10⁶, (y)×10⁷, (y)×10⁸, (y)×10⁹, (y)×10¹⁰, (y)×10¹¹, (y)×10¹², (y)×10¹³, (y)×10¹⁴, or (y)×10¹⁵ cells (or any value from (y)×10⁴ cells to (y)×10¹⁵ cells or range derivable therein) can be electroporated, where y can be any of 1, 2, 3, 4, 5, 6, 7, 8, or 9 (or value from 1 to 9 or range derivable therein), in less than 1, 2, 3, 4, 5, 6, 7, 8, 9, 10, 11, 12, 13, 14, 15, 16, 17, 18, 19, 20, 21, 22, 23, or 24 hours (or any value from 1 hours to 24 hours or range derivable therein).

The expression ‘(y)×10^{e}’ is understood to mean, a variable ‘y’ that can take on any numerical value, multiplied by 10 that is raised to an exponent value, e. For example, (y)×10⁴, where y is 2, is understood to mean 2×10⁴, which is equivalent to 2×10,000, equal to 20,000. (y)×10e4 can also be written as (y)*10e4 or (y)×10⁴ or (y)*10⁴.

Volumes of cells or media may vary depending on the amount of cells to be electroporated, the number of cells to be screened, the type of cells to be screened, the type of protein to be produced, amount of protein desired, cell viability, and certain cell characteristics related to desirable cell concentrations. Examples of volumes that can be used in methods and compositions include, but are not limited to, 0.01, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mL or L (or any value from 0.01 mL or L to 1000 mL or L or range derivable therein), and any range derivable therein. Containers that may hold such volumes are contemplated for use in aspects described herein. Such containers include, but are not limited to, cell culture dishes, petri dishes, flasks, biobags, biocontainers, bioreactors, or vats. Containers for large scale volumes are particularly contemplated, such as those capable of holding greater than 10 L or more. In certain aspects, volumes of 100 L or more are used.

In some aspects, cells are cultured in suspension, using commercially available cell culture vessels and cell culture media. Examples of commercially available culturing vessels that may be used in some aspects including ADME/TOX Plates (GIBCO™), Cell Chamber Slides and Coverslips, Cell Counting Equipment, Cell Culture Surfaces, HYPERFLASK® Cell Culture Vessels (CORNING®), Coated Cultureware, NALGENE® Cryoware, Culture Chamber, Culture Dishes, Glass Culture Flasks, Plastic Culture Flasks, 3D Culture Formats, Culture Multiwell Plates, Culture Plate Inserts, Glass Culture Tubes, Plastic Culture Tubes, Stackable Cell Culture Vessels, Hypoxic Culture Chamber, Petri dish and flask carriers, Quickfit culture vessels, Scale-Up Cell Culture using Roller Bottles, Spinner Flasks, 3D Cell Culture, or cell culture bags.

In other aspects, media may be formulated using components well-known to those skilled in the art. Formulations and methods of culturing cells are described in detail in the following references: Short Protocols in Cell Biology, J. Bonifacino, et al., ed., John Wiley & Sons, 2003, 826 pp; Live Cell Imaging: A Laboratory Manual, D. Spector & R. Goldman, ed., Cold Spring Harbor Laboratory Press, 2004, 450 pp.; Stem Cells Handbook, S. Sell, ed., Humana Press, 2003, 528 pp.; Animal Cell Culture: Essential Methods, John M. Davis, John Wiley & Sons, Mar. 16, 2011; Basic Cell Culture Protocols, Cheryl D. Helgason, Cindy Miller, Humana Press, 2005; Human Cell Culture Protocols, Series: Methods in Molecular Biology, Vol. 806, Mitry, Ragai R.; Hughes, Robin D. (Eds.), 3rd ed. 2012, XIV, 435 p. 89, Humana Press; Cancer Cell Culture: Method and Protocols, Cheryl D. Helgason, Cindy Miller, Humana Press, 2005; Human Cell Culture Protocols, Series: Methods in Molecular Biology, Vol. 806, Mitry, Ragai R.; Hughes, Robin D. (Eds.), 3rd ed. 2012, XIV, 435 p. 89, Humana Press; Cancer Cell Culture: Method and Protocols, Simon P. Langdon, Springer, 2004; Molecular Cell Biology. 4th edition., Lodish H, Berk A, Zipursky S L, et al., New York: W. H. Freeman; 2000, Section 6.2 Growth of Animal Cells in Culture, all of which are incorporated herein by reference.

In some aspects, during the screening and expansion phase and/or during the large scale production phase (also referred to as fed-batch & comparison), expanded electroporated cells that result from selection or screening may comprise an agent of interest.

B. Target Manufacture and Collection

Compositions described herein can be used in therapeutic applications. One example of the therapeutic use of the compositions described herein is formulating a therapeutic agent of interest in appropriate buffer at a required concentration and processing the formulation using systems such as the electroporation systems described herein. If the therapeutic agent of interest and electroporation target are sterile-filtered into a container with an appropriate port(s), the therapeutic agent of interest can be run through a closed, sterile system in a routine laboratory environment. The process can be completed within 2 to 3 hours. Performance variables of the system are generated in real time and can assist quality control operations.

Typically, manufacturing of the agent-loaded target can be done at a central facility or at the point(s)-of care. If there is to be a central facility (or several regional ones), the stability of the agent-loaded target is an important factor. Stability for at least several days could support a custom-order operation. In a point-of-care system, formulated therapeutic agents of interest would be supplied to the sites where therapy is required. Targets or delivery vehicles can be obtained at those sites and the final manufacturing step carried out at processing facilities by technical personnel using detailed standard operating procedures. This would be analogous to final preparation of transfusion products on site. In this case, final product stability is not critical.

C. Therapeutic Applications

The disclosure further encompasses methods for delivering therapeutic agents of interest using an electroporated entity or target, (e.g., cells, cell particles, lipid vesicles, liposomes, tissues, or derivatives thereof) as a delivery vehicle. The present disclosure also includes a method of treating a patient in need of a therapeutic agent of interest comprising administering to the patient an effective amount of cells, cell particles, lipid vesicles, liposomes, or tissues containing the therapeutic agent of interest.

The active agent formulations produced using the methods described herein typically have a sustained effect and lower toxicity, allowing less frequent administration and an enhanced therapeutic index. Therapeutic agents are generated by first preparing cells, cell particles, lipid vesicles, liposomes, tissues, or derivatives thereof loaded with at least one therapeutic agent of interest, obtained according to the methods described herein.

In certain aspects of the present disclosure, agents of interest can be loaded, or introduced, into a delivery vehicle (i.e., an electroporation target, for example, cells, cell particles, lipid vesicles, liposomes, tissues, or derivatives thereof). Examples of suitable agents of interest include, but are not limited to, drugs; stabilizing agents, tracers, fluorescent tags and other imaging substances such as radiolabels; cryoprotectants; nucleic acids; polypeptides; small molecules; carbohydrates; and bioactive materials. Bioactive materials particularly suited to incorporation into electroporation targets include, but are not limited to, therapeutic and prophylactic agents. Examples of bioactive materials include, but are not limited to, proteins and peptides (synthetic, natural, and mimetics), oligonucleotides (anti-sense, ribozymes, etc.), nucleic acids (e.g., double sense linear DNA, inhibitory RNA, siRNA, miRNA, shRNA, expression vectors, etc.), ribonucleoproteins, vectors, small molecules, carbohydrates, cytokines, hemotherapeutic agents, anti-cancer drugs, anti-inflammatory drugs, anti-fungal drugs, anti-viral drugs, anti-microbial drugs, thrombomodulating agents, immunomodulating agents, and the like. It is to be understood that other agents of interest can also be introduced into the delivery vehicle or other cells for delivery to damaged tissue. These agents of interest include, but are not limited to, smooth muscle inhibitors, anti-infective agents (e.g., antibiotics, antifungal agents, antibacterial agents, antiviral agents), chemotherapeutic/antineoplastic agents, and the like.

The agents of interest can be introduced into the delivery vehicle by a variety of methods, with the most preferable method being according to the apparatus and/or methods of the present disclosure. In some aspects, 2, 3, 4, 5, 6, 7, 8, 9, 10, or more agents of interest are serially introduced into the delivery vehicle. In some aspects, the 2, 3, 4, 5, 6, 7, 8, 9, 10, or more agents to be serially introduced into the delivery vehicle may be the same agent, different agents, or a combination thereof. For example, in some aspects, the 2, 3, 4, 5, 6, 7, 8, 9, 10, or more agents to be serially introduced into the delivery vehicle may be the same agent. In some aspects, 2, 3, 4, 5, 6, 7, 8, 9, 10, or more of the agents to be serially introduced into the delivery vehicle may be different agents. In some aspects, the 2, 3, 4, 5, 6, 7, 8, 9, 10, or more agents to be serially introduced into the delivery vehicle may be a combination of the same and different agents (e.g., the second, third, and fourth agent may all be the same agent, while the fifth-tenth agents may be a different agent or a combination of different agents).

In some aspects, the claimed methods of transfecting cells by electroporation, such as flow electroporation, achieve loading, or transfection, efficiencies of an agent of interest of at least, at most, or about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90%, or any range or value derivable therein. The claimed methods of transfecting cells by electroporation, such as flow electroporation, are capable of achieving loading, or transfection, efficiencies of an agent of interest of greater than 40%, greater than 50%, greater than 60%, greater than 70%, greater than 80% or greater than 90% (or any range or value derivable therein). In some aspects, a loading efficiency of an agent of interest is at least 50%, at least 60%, at least 70%, at least 80%, or at least 90%. Transfection efficiency can be measured either by the percentage of the cells that express the product of the gene or the secretion level of the product expressed by the gene.

The dosage of any compositions of the present disclosure will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present disclosure may be readily determined by techniques known to those of skill in the art or as taught herein.

In some aspects, the dosage of the subject compounds can be, can be at most, or can be at least 0.001, 0.010, 0.020, 0.030, 0.040, 0.050, 0.060, 0.070, 0.080, 0.090, 0.100, 0.110, 0.120, 0.130, 0.140, 0.150, 0.160, 0.170, 0.180, 0.190, 0.200, 0.210, 0.220, 0.230, 0.240, 0.250, 0.260, 0.270, 0.280, 0.290, 0.300, 0.310, 0.320, 0.330, 0.340, 0.350, 0.360, 0.370, 0.380, 0.390, 0.400, 0.410, 0.420, 0.430, 0.440, 0.450, 0.460, 0.470, 0.480, 0.490, 0.500, 0.510, 0.520, 0.530, 0.540, 0.550, 0.560, 0.570, 0.580, 0.590, 0.600, 0.610, 0.620, 0.630, 0.640, 0.650, 0.660, 0.670, 0.680, 0.690, 0.700, 0.710, 0.720, 0.730, 0.740, 0.750, 0.760, 0.770, 0.780, 0.790, 0.800, 0.810, 0.820, 0.830, 0.840, 0.850, 0.860, 0.870, 0.880, 0.890, 0.900, 0.910, 0.920, 0.930, 0.940, 0.950, 0.960, 0.970, 0.980, 0.990, 1.000, 1.100, 1.200, 1.300, 1.400, 1.500, 1.600, 1.700, 1.800, 1.900, 2.000, 2.100, 2.200, 2.300, 2.400, 2.500, 2.600, 2.700, 2.800, 2.900, 3.000, 3.100, 3.200, 3.300, 3.400, 3.500, 3.600, 3.700, 3.800, 3.900, 4.000, 4.100, 4.200, 4.300, 4.400, 4.500, 4.600, 4.700, 4.800, 4.900, 5.000, 5.100, 5.200, 5.300, 5.400, 5.500, 5.600, 5.700, 5.800, 5.900, 6.000, 6.100, 6.200, 6.300, 6.400, 6.500, 6.600, 6.700, 6.800, 6.900, 7.000, 7.100, 7.200, 7.300, 7.400, 7.500, 7.600, 7.700, 7.800, 7.900, 8.000, 8.100, 8.200, 8.300, 8.400, 8.500, 8.600, 8.700, 8.800, 8.900, 9.000, 9.100, 9.200, 9.300, 9.400, 9.500, 9.600, 9.700, 9.800, 9.900, or 10.000 pg/ng/mg/g per kg body weight, or any range or value derivable therein. In certain aspects, the dosage of the subject compounds will generally be in the range of about 0.001, 0.01, 1, 5, 10 pg/ng/mg to about 0.1, 1, 5, 10 pg/ng/mg/g per kg body weight, including all values and ranges therebetween.

1. Anti-Infective Agents

In one aspect, the agent of interest is an anti-infective. Anti-infectives are agents that act against infections, such as bacterial, mycobacterial, fungal, viral, or protozoal infections. Anti-infectives covered by the disclosure include, but are not limited to, aminoglycosides (e.g., streptomycin, gentamicin, tobramycin, amikacin, netilmicin, kanamycin, and the like), tetracyclines (e.g., chlortetracycline, oxytetracycline, methacycline, doxycycline, minocycline and the like), sulfonamides (e.g., sulfanilamide, sulfadiazine, sulfamethaoxazole, sulfisoxazole, sulfacetamide, and the like), paraaminobenzoic acid, diaminopyrimidines (e.g., trimethoprim, often used in conjunction with sulfamethoxazole, pyrazinamide, and the like), quinolones (e.g., nalidixic acid, cinoxacin, ciprofloxacin and norfloxacin, and the like), penicillins (e.g., penicillin G, penicillin V, ampicillin, amoxicillin, bacampicillin, carbenicillin, carbenicillin indanyl, ticarcillin, azlocillin, mezlocillin, piperacillin, and the like), penicillinase resistant penicillin (e.g., methicillin, oxacillin, cloxacillin, dicloxacillin, nafcillin, and the like), first generation cephalosporins (e.g., cefadroxil, cephalexin, cephradine, cephalothin, cephapirin, cefazolin, and the like), second generation cephalosporins (e.g., cefaclor, cefamandole, cefonicid, cefoxitin, cefotetan, cefuroxime, cefuroxime axetil, cefmetazole, cefprozil, loracarbef, ceforanide, and the like), third generation cephalosporins (e.g., cefepime, cefoperazone, cefotaxime, ceftizoxime, ceftriaxone, ceftazidime, cefixime, cefpodoxime, ceftibuten, and the like), other beta-lactams (e.g., imipenem, meropenem, aztreonam, clavulanic acid, sulbactam, tazobactam, and the like), betalactamase inhibitors (e.g., clavulanic acid), chlorampheriicol, macrolides (e.g., erythromycin, azithromycin, clarithromycin, and the like), lincomycin, clindamycin, spectinomycin, polymyxin B, polymixins (e.g., polymyxin A, B, C, D, E1 (colistin A), or E2, colistin B or C, and the like), colistin, vancomycin, bacitracin, isoniazid, rifampin, ethambutol, ethionamide, aminosalicylic acid, cycloserine, capreomycin, sulfones (e.g., dapsone, sulfoxone sodium, and the like), clofazimine, thalidomide, and any other antibacterial agent that can be lipid encapsulated.

In certain aspects, anti-microbials include anti-mycobacterials, including, but not limited to, isoniazid, rifampin, streptomycin, rifabutin, ethambutol, pyrazinamide, ethionamide, aminosalicylic, and cycloserine.

Anti-infectives can include antifungal agents, including polyene antifungals (e.g., amphotericin B, nystatin, natamycin, and the like), flucytosine, imidazoles (e.g., n-ticonazole, clotrimazole, econazole, ketoconazole, and the like), triazoles (e.g., itraconazole, fluconazole, and the like), griseofulvin, terconazole, butoconazole ciclopirax, ciclopirox olamine, haloprogin, tolnaftate, naftifine, terbinafine, and any other antifungal that can be lipid encapsulated or complexed. Combinations of drugs can be used.

In certain aspects, anti-infectives include anti-virals, including, but not limited to, anti-herpes agents such as acyclovir, famciclovir, foscamet, ganciclovir, acyclovir, idoxuridine, sorivudine, trifluridine, valacyclovir and vidarabine; anti-retroviral agents such as ritonavir, didanosine, stavudine, zalcitabine, tenovovir and zidovudine; and other antiviral agents such as, but not limited to, amantadine, interferon-alpha, ribavirin, and rimantadine.

Also included as suitable anti-infectives used in the formulations of the present disclosure are pharmaceutically acceptable addition salts and complexes of drugs. In cases wherein the compounds may have one or more chiral centers, unless specified, the present disclosure comprises each unique racemic compound, as well as each unique non-racemic compound.

2. Anti-Neoplastic Agents

In one aspect, an active agent is an antineoplastic drug. Currently, there are approximately twenty recognized classes of approved antineoplastic drugs. The classifications are generalizations based on either a common structure shared by particular drugs, or are based on a common mechanism of action by the drugs. A partial listing of some of the commonly known antineoplastic agents by classification is as follows:

Structure-based classes include Fluoropyrimidines-5-FU, Fluorodeoxyuridine, Ftorafur, 5′-deoxyfluorouridine, UFT, S-1 Capecitabine; Pyrimidine Nucleosides—Deoxycytidine, Cytosine Arabinoside, 5-Azacytosine, Gemcitabine, 5-Azacytosine-Arabinoside; Purines—6-Mercaptopurine, Thioguanine, Azathioprine, Allopurinol, Cladribine, Fludarabine, Pentostatin, 2-Chloro Adenosine; Platinum Analogues—Cisplatin, Carboplatin, Oxaliplatin, Tetraplatin, Platinum-DACH, Ormaplatin, CI-973, JM-216; Anthracyclines/Anthracenediones—Doxorubicin, Daunorubicin, Epirubicin, Idarubicin, Mitoxantrone; Epipodophyllotoxins—Etoposide, Teniposide; C amptothecins—Irinotec an, Topotecan, 9-Amino Camptothecin, 10,11-Methylenedioxy Camptothecin, 9-Nitro Camptothecin, TAS 103, 7-(4-methyl-piperazino-methylene)-10,11-ethylenedioxy-20(S)-camptothecin, 7-(2-N-isopropylamino)ethyl)-20(S)-camptothecin; Hormones and Hormonal Analogues—Diethylstilbestrol, Tamoxifen, Toremefine, Tolmudex, Thymitaq, Flutamide, Bicalutamide, Finasteride, Estradiol, Trioxifene, Droloxifene, Medroxyprogesterone Acetate, Megesterol Acetate, Aminoglutethimide, Testolactone and others; Enzymes, Proteins and Antibodies—Asparaginase, Interleukins, Interferons, Leuprolide, Pegaspargase, and others; Vinca Alkaloids—Vincristine, Vinblastine, Vinorelbine, Vindesine; Taxanes—Paclitaxel, and Docetaxel.

Mechanism-based classes include Antihormonals—Anastrozole; Antifolates—Methotrexate, Aminopterin, Trimetrexate, Trimethoprim, Pyritrexim, Pyrimethamine, Edatrexate, MDAM; Antimicrotubule Agents—Taxanes and Vinca Alkaloids; Alkylating Agents (Classical and Non-Classical)-Nitrogen Mustards (Mechlorethamine, Chlorambucil, Melphalan, Uracil Mustard), Oxazaphosphorines (Ifosfamide, Cyclophosphamide, Perfosfamide, Trophosphamide), Alkylsulfonates (Busulfan), Nitrosoureas (Carmustine, Lomustine, Streptozocin), Thiotepa, Dacarbazine and others; Antimetabolites—Purines, pyrimidines and nucleosides, listed above; Antibiotics—Anthracyclines/Anthracenediones, Bleomycin, Dactinomycin, Mitomycin, Plicamycin, Pentostatin, Streptozocin; Topoisomerase Inhibitors—Camptothecins (Topo I), Epipodophyllotoxins, m-AMS A, Ellipticines (Topo II); Antivirals—AZT, Zalcitabine, Gemcitabine, Didanosine, and others; Miscellaneous Cytotoxic Agents—siRNA, miRNA, Hydroxyurea, Mitotane, Fusion Toxins, PZA, Bryostatin, Retinoids, Butyric Acid and derivatives, Pentosan, Fumagillin, and others.

3. Anti-Angiogenic Agents

Antiangiogenic agents can be incorporated into the electroporation targets. Antiangiogenic drugs include, but are not limited to, AGM-1470 (TNP-470) or antagonists to one of its receptors, MetAP-2; growth factor antagonists, or antibodies to growth factors (including VEGF or bFGF); growth factor receptor antagonists or antibodies to growth factor receptors; inhibitors of metalloproteinases including TIMP, batimastat (BB-94), and marimastat; tyrosine kinase inhibitors including genistein and SU5416; integrin antagonists including antagonists alphaVbeta3/5 or antibodies to integrins; retinoids including retinoic acid or the synthetic retinoid fenretinide; steroids 11α-epihydrocortisol, corteloxone, tetrahydrocortisone and 17α-hydroxyprogesterone; protein kinase inhibitors including staurosporine and MDL 27032; vitamin D derivatives including 22-oxa-1 alpha, and 25-dihydroxyvitamin D3; arachidonic acid inhibitors including indomethacin and sulindac; tetracycline derivatives including minocycline; thalidomide and thalidomide analogs and derivatives; 2-methoxyestradiol; tumor necrosis factor-alpha; interferon-gamma-inducible protein 10 (IP-10); interleukin 1 and interleukin 12; interferon alpha, beta or gamma; angiostatin protein or plasminogen fragments; endostatin protein or collagen 18 fragments; proliferin-related protein; group B streptococcus toxin; CM101; CAI; troponin I; squalamine; nitric oxide synthase inhibitors including L-NAME; thrombospondin; wortmannin; amiloride; spironolactone; ursodeoxycholic acid; bufalin; suramin; tecogalan sodium; linoleic acid; captopril; irsogladine; FR-118487; triterpene acids; castanospermine; leukemia inhibitory factor; lavendustin A; platelet factor-4; herbimycin A; diaminoantraquinone; taxol; aurintricarboxylic acid; DS-4152; pentosan polysulphite; radicicol; fragments of human prolactin; erbstatin; eponemycin; shark cartilage; protamine; Louisianin A, C and D; PAF antagonist WEB 2086; auranofin; ascorbic ethers; and sulfated polysaccharide D 4152.

4. Biomolecular Agents

Genes to be targeted with nucleic acid agents using the methods of the disclosure include, without limitation, those whose expression is correlated with an undesired phenotypic trait. Thus, genes relating to cancer and viruses, for example, can be targeted. Cancer-related genes include oncogenes (e.g., K-ras, c-myc, bcr/abl, c-myb, c-fms, c-fos and cerb-B), growth factor genes (e.g., genes encoding epidermal growth factor and its receptor, fibroblast growth factor-binding protein), matrix metalloproteinase genes (e.g., the gene encoding MMP-9), adhesion-molecule genes (e.g., the gene encoding VLA-6 integrin), tumor suppressor genes (e.g., bcl-2 and bcl-X1), angiogenesis genes, and metastatic genes. Viral genes include human papilloma virus genes (related, for example, to cervical cancer), hepatitis B and C genes, and cytomegalovirus (CMV) genes (related, for example, to retinitis). Numerous other genes relating to these diseases or others might also be targeted. In certain aspects nucleic acids can target mRNA encoding c-myc, VEGF, CD4, CCRS, gag, MDM2, Apex, Ku70, or ErbB2.

Methods of gene regulation include administering siRNA, miRNA, shRNA, antisense oligonucleotides, and other inhibitory nucleic acids and/or administration of vectors or nucleic acids encoding a therapeutic polynucleotide, protein, ribonucleoprotein, or peptide. In another aspect, the disclosure provides a method of preparing and/or administering to the subject a dosage of a therapeutic inhibitory oligonucleotide or nucleic acid (antisense oligonucleotide, ribozyme, siRNA, shRNA, miRNA, dsRNA) molecule, wherein the administered nucleic acid inhibits a biological process such as transcription or translation. The present disclosure provides methods of administering one or more therapeutic nucleic acid molecules to a subject, using a nucleic acid delivery vehicle prepared using the methods described, to bring about a therapeutic benefit to a subject. As used herein, a “therapeutic nucleic acid molecule” or “therapeutic nucleic acid” is any nucleic acid (e.g., DNA, RNA, non-naturally occurring nucleic acids and their analogues such as peptide nucleic acids, and their chemical conjugates) that, as a nucleic acid or as an expressed nucleic acid or polypeptide, confers a therapeutic benefit to a subject. The subject can be mammalian, for example, a mouse, or a human being.

Methods of gene regulation also include gene editing of cells to remove 1, 2, 3, 4, 5, 6, 7, 8, 9, 10, or more endogenous genes in the cells. Methods of gene editing include, but are not limited to, RNA-guided endonucleases (RGENs) (e.g., ribonucleoproteins), restriction enzymes, zinc finger nucleases (ZFNs), and transcription activator-like effector nucleases (TALENs). In particular aspects, one or more endogenous genes of the cells are modified, such as disrupted in expression where the expression is reduced in part or in full. In specific aspects, one or more genes are knocked down or knocked out using processes of the disclosure. Disruption of gene expression or gene knockout or knockdown may be accomplished by electroporating cells to introduce one or more RGENs, restriction enzymes, ZFNs, or TALENs, according to the electroporation apparatus and/or methods of the present disclosure. In some aspects, the cells are sequentially electroporated to permit serial editing of the cells as one or more RGENs, restriction enzymes, ZFNs, or TALENs are sequentially introduced to sequentially disrupt, knock out, or knock down 2, 3, 4, 5, 6, 7, 8, 9, 10, or more genes. The genes that are edited in the cells may be of any kind, but in specific aspects the genes are genes whose gene products is correlated with an undesired phenotypic trait, as described herein.

a. Nucleic Acids

Aspects concern electroporating cells, cell particles, lipid vesicles, liposomes, tissues, or derivatives thereof with a composition comprising a therapeutic nucleic acid. In certain aspects, the nucleic acid molecule can be in the form of an oligonucleotide.

The term “oligo” or “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the aspect being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, shRNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this disclosure that is a polynucleotide encompasses both the double-stranded form and each of two complementary single-stranded forms known or predicted to make up the double-stranded form.

The DNA oligonucleotide may be from at least, at most, or about 10, 20, 25, 30, 35, 40, 50, 60, 70, 80, 90, 100, 150, 200, 250, 300, 350, 400, 450, 500, 550, or 600 nucleotides to at least, at most, or about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3250, 3500, 3750, 4000, 4250, 4500, 4750, or 5000 nucleotides in length, or any value from 10 nucleotides to 5000 nucleotides or derivable range thereof. In certain aspects, the oligonucleotide is more than 10 nucleotides, or more than 11, 12, 13, 14, 15, 16, 17, 18, 19, 20, 21, 22, 23, 24, 25, 30, or 40 nucleotides. In specific aspects, the oligonucleotide is from about 30 to about 300 nucleotides, from about 20 to about 200 nucleotides, from about 15 to about 150 nucleotides, from about 10 to about 100 nucleotides, or from about 40 to about 100 nucleotides. In certain aspects, the oligonucleotide, regardless of the length of a coding sequence, may be combined with other nucleic acid sequences, such as promoters, polyadenylation signals, restriction enzyme sites, multiple cloning sites, other coding segments, and the like, such that their overall length may vary considerably.

The concentration of the oligonucleotide during the electroporation procedure may be the final concentration of the oligonucleotide in the electroporation chamber and/or sample container. The oligonucleotide concentration may be from at least, at most, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 50, 75, 100, 150, 200, 250, or 300 to about 350, 400, 500, 1000, 1500, 2000, 3000, 4000, or 5000 μg/mL, or any value from 0.01 μg/mL to 5000 μg/mL or range derivable therein. In certain aspects, the oligonucleotide concentration is at least 1 μg/mL. In further aspects, the concentration of the oligonucleotide is at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 m/mL, or any value from 1 μg/mL to 300 m/mL or range derivable therein.

In the context of this disclosure, the term “unmodified oligonucleotide” refers generally to an oligomer or polymer of ribonucleic acid (RNA) or deoxyribonucleic acid (DNA). In some aspects, a nucleic acid molecule is an unmodified oligonucleotide. This term includes oligonucleotides composed of naturally occurring nucleobases, sugars, and covalent internucleoside linkages. The term “oligonucleotide analog” refers to oligonucleotides that have one or more non-naturally occurring portions that function in a similar manner to oligonucleotides. Such non-naturally occurring oligonucleotides are often selected over naturally occurring forms because of desirable properties such as, for example, enhanced cellular uptake, enhanced affinity for other oligonucleotides or nucleic acid targets, and increased stability in the presence of nucleases. The term “oligonucleotide” can be used to refer to unmodified oligonucleotides or oligonucleotide analogs.

Specific examples of nucleic acid molecules include nucleic acid molecules containing modified, i.e., non-naturally occurring internucleoside linkages. Such non-naturally internucleoside linkages are often selected over naturally occurring forms because of desirable properties such as, for example, enhanced cellular uptake, enhanced affinity for other oligonucleotides or nucleic acid targets, and increased stability in the presence of nucleases. In a specific aspect, the modification comprises a methyl group.

Nucleic acid molecules can have one or more modified internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Modifications to nucleic acid molecules can include modifications wherein one or both terminal nucleotides is modified.

One suitable phosphorus-containing modified internucleoside linkage is the phosphorothioate internucleoside linkage. A number of other modified oligonucleotide backbones (internucleoside linkages) are known in the art and may be useful in the context of this aspect. Representative U.S. patents that teach the preparation of phosphorus-containing internucleoside linkages include, but are not limited to, U.S. Pat. Nos. 3,687,808; 4,469,863; 4,476,301; 5,023,243, 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697; 5,625,050; 5,489,677; and 5,602,240, each of which is incorporated herein by reference.

Modified oligonucleoside backbones (internucleoside linkages) that do not include a phosphorus atom therein have internucleoside linkages that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having amide backbones; and others, including those having mixed N, O, S and CH2 component parts. Representative U.S. patents that teach the preparation of the above non-phosphorous-containing oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269; and 5,677,439, each of which is incorporated herein by reference.

Oligomeric compounds can also include oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring with for example a morpholino ring, is also referred to in the art as being a sugar surrogate. The heterocyclic base moiety or a modified heterocyclic base moiety is maintained for hybridization with an appropriate target nucleic acid. Oligonucleotide mimetics can include oligomeric compounds such as peptide nucleic acids (PNA) and cyclohexenyl nucleic acids (known as CeNA, see Wang et al., J. Am. Chem. Soc., 2000, 122, 8595-8602). Representative U.S. patents that teach the preparation of oligonucleotide mimetics include, but are not limited to, U.S. Pat. Nos. 5,539,082; 5,714,331; and 5,719,262, each of which is incorporated herein by reference. Another class of oligonucleotide mimetic is referred to as phosphonomonoester nucleic acid and incorporates a phosphorus group in the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology. Another oligonucleotide mimetic has been reported wherein the furanosyl ring has been replaced by a cyclobutyl moiety.

Nucleic acid molecules can also contain one or more modified or substituted sugar moieties. The base moieties are maintained for hybridization with an appropriate nucleic acid target compound. Sugar modifications can impart nuclease stability, binding affinity or some other beneficial biological property to the oligomeric compounds.

Representative modified sugars include carbocyclic or acyclic sugars, sugars having substituent groups at one or more of their 2′, 3′ or 4′ positions, sugars having substituents in place of one or more hydrogen atoms of the sugar, and sugars having a linkage between any two other atoms in the sugar. A large number of sugar modifications are known in the art, sugars modified at the 2′ position and those which have a bridge between any 2 atoms of the sugar (such that the sugar is bicyclic) are particularly useful in this aspect. Examples of sugar modifications useful in this aspect include, but are not limited to compounds comprising a sugar substituent group selected from: OH; F; O—, S—, or N-alkyl; or O-alkyl-O-alkyl, wherein the alkyl, alkenyl and alkynyl may be substituted or unsubstituted C1 to C10 alkyl or C2 to C10 alkenyl and alkynyl. Particularly suitable are: 2-methoxyethoxy (also known as 2′-O-methoxyethyl, 2′-MOE, or 2′-OCH2CH2OCH3), 2′-0-methyl (2′-O—CH3), 2′-fluoro (2′-F), or bicyclic sugar modified nucleosides having a bridging group connecting the 4′ carbon atom to the 2′ carbon atom wherein example bridge groups include —CH2—O—, —(CH2)2—O— or —CH2-N(R3)—O wherein R3 is H or C1-C12 alkyl.

One modification that imparts increased nuclease resistance and a very high binding affinity to nucleotides is the 2′-MOE side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-MOE substituent also have been shown to be antisense inhibitors of gene expression with promising features for in vivo use (Martin, P., Hely. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

2′-Sugar substituent groups may be in the arabino (up) position or ribo (down) position. One 2′-arabino modification is 2′-F. Similar modifications can also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is incorporated herein by reference in its entirety.

Representative sugar substituents groups are disclosed in U.S. Pat. No. 6,172,209 entitled “Capped 2′-Oxyethoxy Oligonucleotides,” hereby incorporated by reference in its entirety. Representative cyclic sugar substituent groups are disclosed in U.S. Pat. No. 6,271,358 entitled “RNA Targeted 2′-Oligomeric compounds that are Conformationally Preorganized,” hereby incorporated by reference in its entirety. Representative guanidino substituent groups are disclosed in U.S. Pat. No. 6,593,466 entitled “Functionalized Oligomers,” hereby incorporated by reference in its entirety. Representative acetamido substituent groups are disclosed in U.S. Pat. No. 6,147,200 which is hereby incorporated by reference in its entirety.

Nucleic acid molecules can also contain one or more nucleobase (often referred to in the art simply as “base”) modifications or substitutions which are structurally distinguishable from, yet functionally interchangeable with, naturally occurring or synthetic unmodified nucleobases. Such nucleobase modifications can impart nuclease stability, binding affinity or some other beneficial biological property to the oligomeric compounds. As used herein, “unmodified” or “natural” nucleobases include the purine bases adenine (A) and guanine (G), and the pyrimidine bases thymine (T), cytosine (C) and uracil (U). Modified nucleobases also referred to herein as heterocyclic base moieties include other synthetic and natural nucleobases, many examples of which such as 5-methylcytosine (5-me-C), 5-hydroxymethyl cytosine, 7-deazaguanine and 7-deazaadenine among others.

Heterocyclic base moieties can also include those in which the purine or pyrimidine base is replaced with other heterocycles, for example 7-deaza-adenine, 7-deazaguanosine, 2-aminopyridine and 2-pyridone. Some nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds. These include 5-substituted pyrimidines, 6-azapyrimidines and N-2, N-6 and O-6 substituted purines, including 2 aminopropyladenine, 5-propynyluracil and 5-propynylcytosine.

Additional modifications to nucleic acid molecules are disclosed in U.S. Patent Publication 2009/0221685, which is hereby incorporated by reference. Also disclosed herein are additional suitable conjugates to the nucleic acid molecules.

b. Proteins

Aspects concern electroporating cells, cell particles, lipid vesicles, liposomes, tissues, or derivatives thereof with a composition comprising a therapeutic protein or peptide.

As used herein, a “protein” or “peptide” or “polypeptide” refers to a molecule comprising at least two amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects, protein or polypeptide size (wild-type or modified) may comprise, but is not limited to, at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, or any value from 1 amino acid to 2500 amino acids or range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

Nucleotide as well as protein, polypeptide, and peptide sequences for various genes have been previously disclosed, and may be found in the recognized computerized databases. Two commonly used databases are the National Center for Biotechnology Information's GENEBANK® and GENPEPT® databases (on the World Wide Web at ncbi.nlm.nih.gov) and The Universal Protein Resource (UNIPROT®; on the World Wide Web at uniprot.org). The coding regions for these genes may be electroporated using the techniques disclosed herein or as would be known to those of ordinary skill in the art.

The concentration of the protein or polypeptide during the electroporation procedure may be the final concentration of the protein in the electroporation chamber and/or sample container. The concentration of a polypeptide during an electroporation procedure may be from at least, at most, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 50, 75, 100, 150, 200, 250, 300 to at least, at most, or about 350, 400, 500, 1000, 1500, 2000, 3000, 4000, or 5000 μg/mL, or any value from 0.01 μg/mL to 5000 μg/mL or range derivable therein. In certain aspects, the concentration of the polypeptide is at least 1 μg/mL. In further aspects, the concentration of the polypeptide is at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 μg/mL, or any value from 1 μg/mL to 300 μg/mL or range derivable therein.

Proteins of the disclosure may also comprise alternate amino acid subunits of the protein as compared to the wild-type protein to create an equivalent, or even improved, second-generation variant polypeptides or peptides. Since it is the interactive capacity and nature of a protein that defines its functional activity, certain amino acid substitutions can be made in a protein sequence and in its corresponding DNA coding sequence, and nevertheless produce a protein with similar or desirable properties.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to the wild-type protein sequence. A variant can include 2, 3, 4, 5, 6, 7, 8, 9, 10, 11, 12, 13, 14, 15, 16, 17, 18, 19, 20, or more substitute amino acids, for example.

It also will be understood that amino acid and nucleic acid sequences may include additional residues, such as additional N- or C-terminal amino acids, or 5′ or 3′ nucleic acid sequences, respectively, and yet still be essentially identical to the wild-type sequence, so long as the sequence maintains biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins, which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative” (also “nonconservative”). In some aspects, a non-conservative substitution affects a function or activity of the polypeptide. In some aspects, a non-conservative substitution does not affect a function or activity of the polypeptide. Non-conservative changes typically involve substituting an amino acid residue with one that is chemically dissimilar, such as a polar or charged amino acid for a nonpolar or uncharged amino acid, and vice-versa. Non-conservative substitutions may involve the exchange of a member of one of the amino acid classes for a member from another class.

c. Ribonucleoproteins

Aspects concern electroporating cells with a composition comprising one or more DNA-binding nucleic acids, such as alteration via an RNA-guided endonuclease (RGEN) (e.g., a ribonucleoprotein) for gene editing of the cells. In certain aspects, the ribonucleoprotein comprises clustered regularly interspaced short palindromic repeats (CRISPR) and CRISPR-associated (Cas) proteins.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 13, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other aspects, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other aspects, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. The tracr sequence, which may comprise or consist of all or a portion of a wild-type tracr sequence (e.g., at most, at least, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell (e.g., by electroporation) such that expression of the elements of the CRISPR system direct formation of the CRISPR complex at one or more target sites. Components can also be delivered to cells as proteins and/or RNA and/or ribonucleoproteins. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some aspects, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SWISSPROT® database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some aspects, a Cas9 nickase may be used in combination with guide sequence(s), e.g., two guide sequences, which target respectively sense and antisense strands of the DNA target. This combination allows both strands to be nicked and used to induce NHEJ or HDR.

In some aspects, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some aspects, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies), ELAND (ILLUMINA®, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

d. Vectors

Therapeutic polynucleotides, proteins, ribonucleoproteins, or peptides may be encoded by a nucleic acid molecule in the composition. In certain aspects, the nucleic acid molecule can be in the form of a nucleic acid vector.

The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art will be well equipped to construct a vector through standard recombinant techniques (for example Sambrook et al., 2001; Ausubel et al., 1996, both incorporated herein by reference).

The term “expression vector” refers to a vector containing a nucleic acid sequence coding for at least part of a gene product capable of being transcribed or stably integrated into a host cell's genome and subsequently be transcribed. In some cases, nucleic acid molecules are then translated into a protein, polypeptide, or peptide. To express the polynucleotides, proteins, ribonucleoproteins, or peptides, DNA encoding the polynucleotides, proteins, ribonucleoproteins, or peptides are inserted into expression vectors such that the gene area is operatively linked to transcriptional and translational “control sequences.” Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

The particular promoter that is employed to control the expression of a peptide or protein encoding polynucleotide is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

A specific initiation signal also may be required for efficient translation of coding sequences. These signals include the ATG initiation codon or adjacent sequences. Exogenous translational control signals, including the ATG initiation codon, may need to be provided. One of ordinary skill in the art will readily be capable of determining this and providing the necessary signals.

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

The vectors or constructs will generally comprise at least one termination signal. A “termination signal” or “terminator” is comprised of the DNA sequences involved in specific termination of an RNA transcript by an RNA polymerase. Thus, in certain aspects, a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other aspects involving eukaryotes, it is preferred that the terminator comprises a signal for the cleavage of the RNA, and it is more preferred that the terminator signal promotes polyadenylation of the message. In gene expression, particularly eukaryotic gene expression, one will typically include a polyadenylation signal to effect proper polyadenylation of the transcript.

In order to propagate a vector in a host cell, the vector may also contain one or more origins of replication sites (often termed “ori”), which is a specific nucleic acid sequence at which replication is initiated. Alternatively, an autonomously replicating sequence (ARS) can be employed if the host cell is yeast.

Some vectors may employ control sequences that allow the vector to be replicated and/or expressed in both prokaryotic and eukaryotic cells. One of skill in the art will further understand the conditions under which to incubate all of the above described host cells to maintain them and to permit replication of a vector. Also understood and known are techniques and conditions that will allow large-scale production of vectors, as well as production of the nucleic acids encoded by vectors and their cognate polypeptides, proteins, or peptides.

The concentration of the vector during the electroporation procedure may be the final concentration of the vector in the electroporation chamber and/or sample container. The vector concentration can be, can be at least, or can be at most 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 50, 75, 100, 150, 200, 250, 300 to about 350, 400, 500, 1000, 1500, 2000, 3000, 4000, 5000 μg/mL, or any value from 0.01 μg/mL to 5000 μg/mL or range derivable therein. In certain aspects, the concentration of the vector is at least 10 μg/mL. In further aspects, the concentration of the vector is at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 μg/mL, or any value from 1 μg/mL to 300 μg/mL or range derivable therein.

It is contemplated that expression vectors that express a marker may be useful in the disclosure. In other aspects, the marker is encoded on an mRNA and not in an expression vector.

In certain specific aspects, the composition transfected into the delivery vehicle by electroporation is non-viral (i.e., does not contain any viral components). It is contemplated that non-viral methods can reduce toxicity and/or improve the safety of the method.

e. Markers

In certain aspects, cells, cell particles, lipid vesicles, liposomes, tissues, or derivatives thereof that have been transfected with a composition of the present disclosure may be identified in vitro or in vivo by including a marker in the composition. Such markers would confer an identifiable change to the cell, permitting easy identification of cells that have been transfected with the composition.

Generally, a selectable marker is one that confers a property that allows for selection. A positive selectable marker is one in which the presence of the marker allows for its selection, while a negative selectable marker is one in which its presence prevents its selection. In certain aspects, after electroporation, delivery vehicles that have internalized the electroporated compositions are selected for by negative selection. In other aspects, after electroporation, cells that have internalized the electroporated constructs are selected for by positive selection.

An example of a positive selectable marker is a drug resistance marker or an antibiotic resistance gene/marker. Usually, the inclusion of a drug selection marker aids in the cloning and identification of transformants; for example, genes that confer resistance to neomycin, puromycin, hygromycin, DHFR, GPT, zeocin, G418, phleomycin, blasticidin, and histidinol are useful selectable markers.

In some aspects, selection involves exposing the cells to concentrations of a selection agent that will compromise the viability of a cell that does not express a selection resistance gene or take up a selection resistance gene during electroporation. In some aspects, selection involves exposing the cells to a conditionally lethal concentration of the selection agent. In certain aspects, the selection agent or compound is an antibiotic. In other aspects, the selection agent is G418 (also known as geneticin and G418 sulfate), puromycin, zeocin, hygromycin, phleomycin or blasticidin, either alone or in combination. In certain aspects, the concentration of selection agent can be, can be at least, or can be at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μg/L, mg/L, or g/L, or any range or value derivable therein. In certain aspects, the concentration of selection agent is in the range of 0.1 μg/L to 0.5 μg/L, 0.5 μg/L to 1 μg/L, 1 μg/L to 2 μg/L, 2 μg/L to 5 μg/L, 5 μg/L to 10 μg/L, 10 μg/L to 100 μg/L, 100 μg/L to 500 μg/L, 0.1 mg/L to 0.5 mg/L, 0.5 mg/L to 1 mg/L, 1 mg/L to 2 mg/L, 2 mg/L to 5 mg/L, 5 mg/L to 10 mg/L, 10 mg/L to 100 mg/L, 100 mg/L to 500 mg/L, 0.1 g/L to 0.5 g/L, 0.5 g/L to 1 g/L, 1 g/L to 2 g/L, 2 g/L to 5 g/L, 5 g/L to 10 g/L, 10 g/L to 100 g/L, or 100 g/L to 500 g/L, or any value from 0.1 μg/L to 500 g/L or range derivable therein. In certain aspects, the concentration of selection agent is (y)g/L, where ‘y’ can be any value including but not limited to 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, or any value from 0.01 to 100 or range derivable therein. In some aspects, the selection agent is present in the culture media at a conditionally lethal concentration of at least, at most, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 g/L, or any value from 0.1 to g/L to 10 g/L or range derivable therein.

In addition to markers conferring a phenotype that allows for the discrimination of transformants based on the implementation of conditions, other types of markers including screenable markers, such as GFP, are also contemplated. In certain aspects, the marker is a fluorescent marker, an enzymatic marker, a luminescent marker, a photoactivatable marker, a photoconvertible marker, or a colorimetric marker. Fluorescent markers include, for example, GFP and variants such as YFP, RFP etc., and other fluorescent proteins such as DsRed, mPlum, mCherry, YPet, Emerald, CyPet, T-Sapphire, and Venus. Photoactivatable markers include, for example, KFP, PA-mRFP, and Dronpa. Photoconvertible markers include, for example, mEosFP, KikGR, and PS-CFP2. Luminescent proteins include, for example, Neptune, FP595, and phialidin. Alternatively, screenable enzymes such as herpes simplex virus thymidine kinase (tk) or chloramphenicol acetyltransferase (CAT) may be utilized. One of skill in the art will also know how to employ immunologic markers, possibly in conjunction with FACS analysis. Further examples of selectable and screenable markers are well known to one of skill in the art.

The marker used may be encoded on an RNA or DNA. In some aspects, the marker is encoded on RNA.

### EXAMPLES

The following examples are included to demonstrate preferred aspects of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific aspects that are disclosed and still obtain a like or similar result without departing from the spirit and scope of the disclosure.

### Example 1—Repeated Electroporation of Expanded T Cells with mRNA

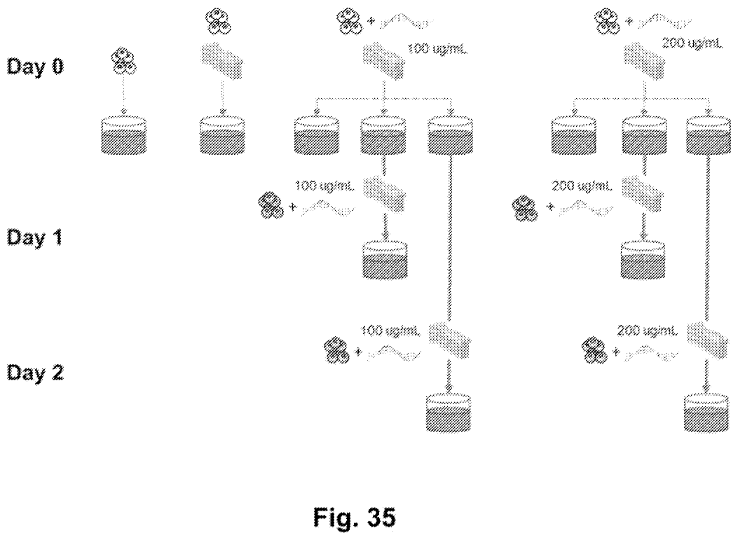

FIG. 35 illustrates an experimental design for sequential electroporation of expanded lymphocytes with two different GFP mRNA concentrations (100 μg/mL and 200 μg/mL). On Day 0, after being expanded for 3 days with CD3/CD28 beads, 5×10⁷ T cells/mL were suspended in electroporation (EP) buffer. Electroporation was performed +/− green fluorescent protein (GFP) mRNA, and cells were plated. On Day 1 or Day 2, GFP-expressing cells were resuspended in EP buffer and electroporated a second time with GFP, and cells were plated. Cell viability was measured via Nucleocounter on Days 1-5, and GFP expression was assayed via flow cytometry on Days 3 and 4.

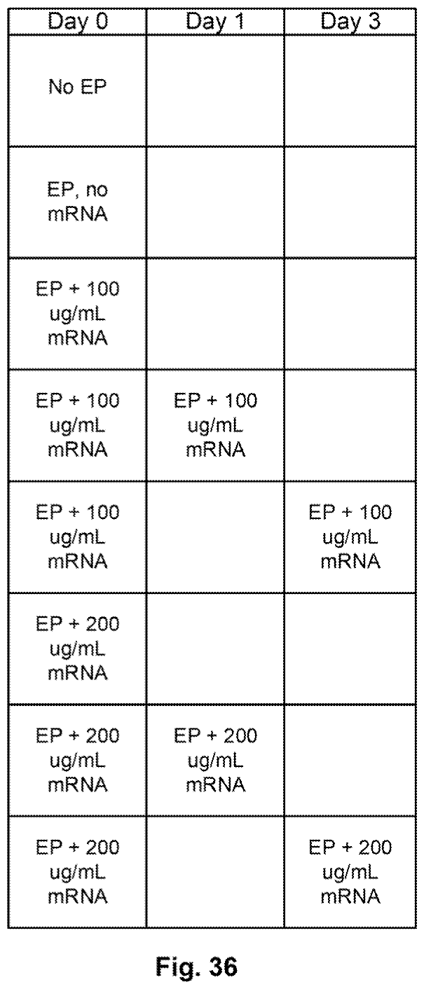

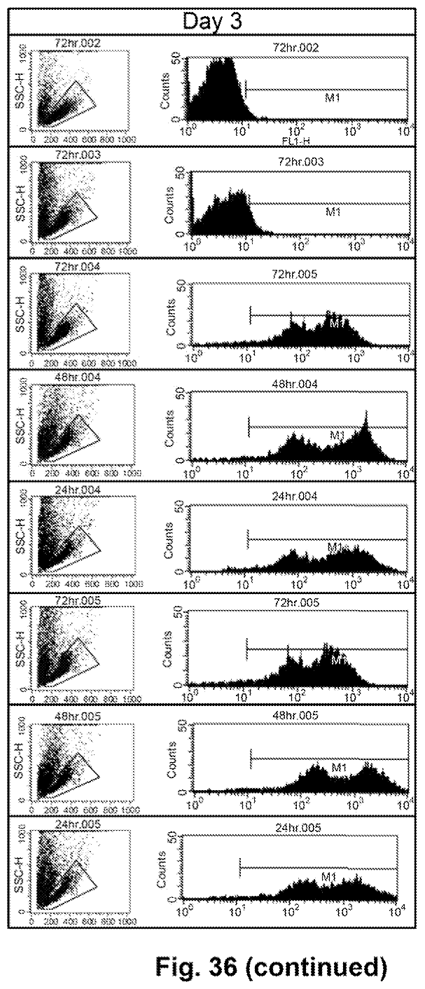

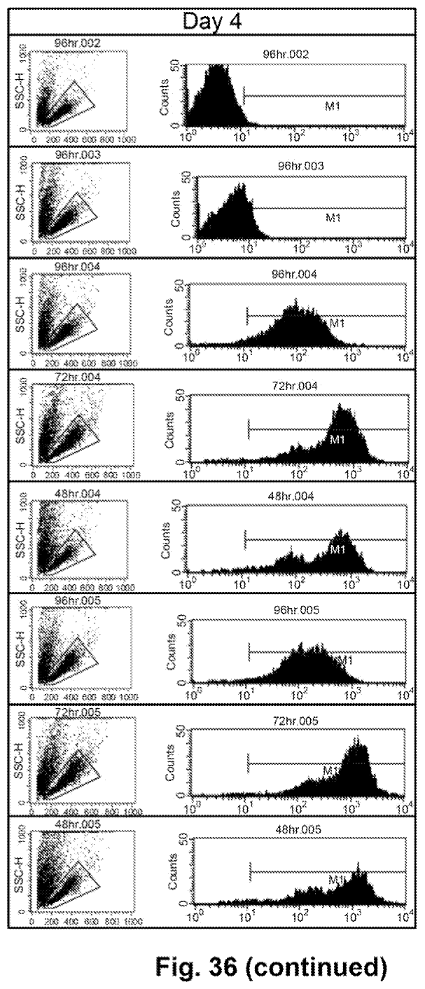

FIG. 36 shows flow cytometry data from Days 3 and 4 after sequential electroporation of expanded lymphocytes cells with GFP mRNA. All mRNA transfected cells were >95% GFP+.

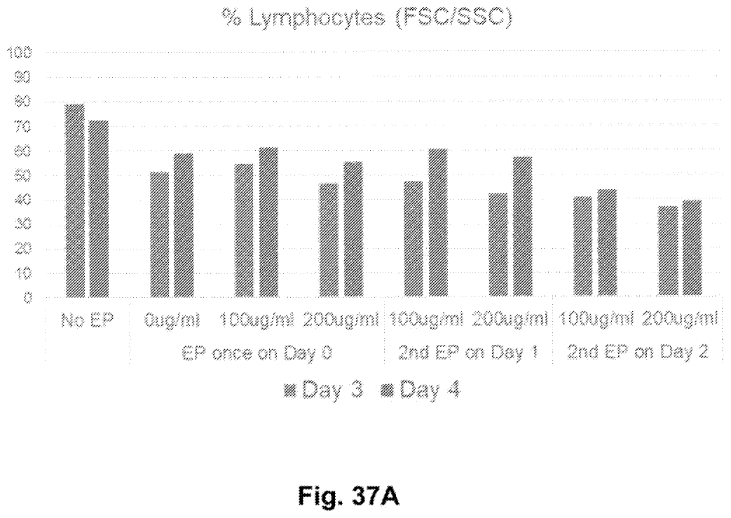

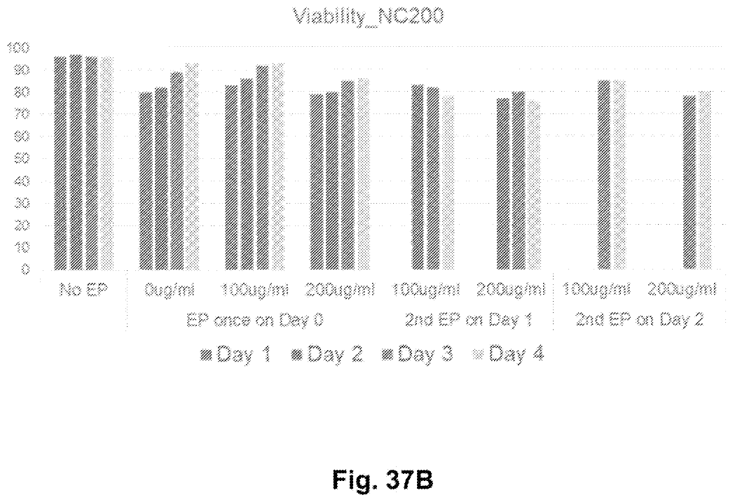

FIGS. 37A-37B show lymphocyte gating and cell viability of lymphocytes subjected to sequential electroporation. Sequential electroporation with or without mRNA on consecutive days produced minimal impact on cell fitness as assessed by percentage of lymphocytes and dye exclusion. Increasing the gap between EP pulses to 48 hours (sequential electroporation of Day 0 and Day 2) resulted in slightly lower cell fitness compared to single EP or sequential EP on Day 0 and Day 1.

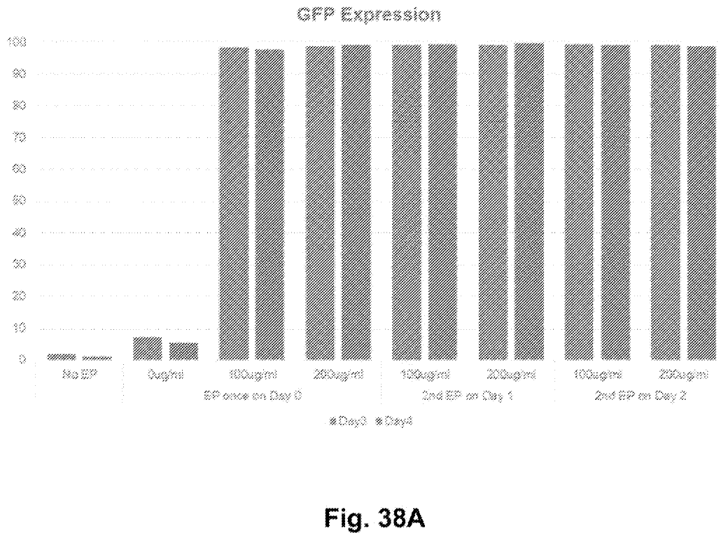

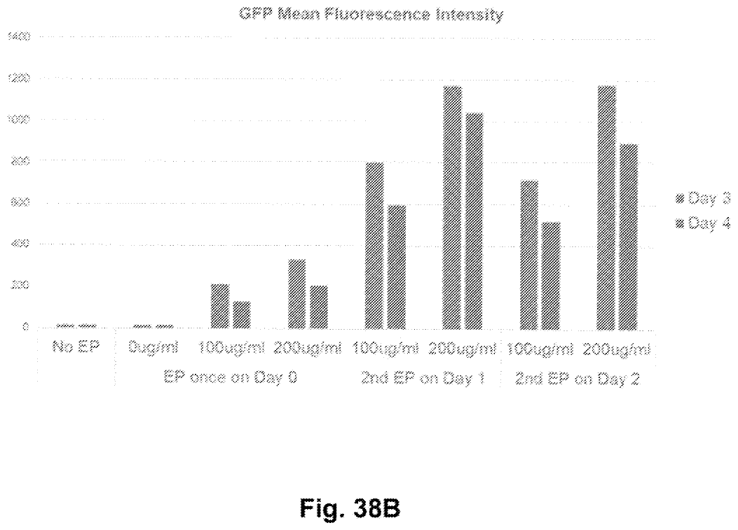

FIG. 38A shows GFP expression by sequentially electroporated lymphocytes and FIG. 38B shows GFP mean fluorescence intensity (MFI) for sequentially electroporated lymphocytes. Sequential EP of mRNA significantly increased the level and duration of transgene expression compared to a single EP.

### Example 2—Repeated Electroporation of Expanded T cells with mRNA, Effect of Electroporation Energy

For the experiments described herein, the term “energy” refers to the heat produced during an electrical pulse (or combined pulses) applied to a sample, and it is proportional to both the field strength and the pulse duration (or combined pulse duration) applied to the sample during the electrical pulse (or combined pulses). Thus, to apply a “high energy” pulse to a sample, the proportions of variables including field strength and pulse duration (or combined pulse duration) are modified such that a greater amount of heat is produced during the electrical pulse (or combined pulses) compared to when a “medium energy” or a “low energy” electrical pulse (or combined pulses) is applied to the sample, provided the buffer composition, the processing assembly, and the sample volume are held constant. Conversely, to apply a “low energy” pulse to a sample, the proportions of variables including field strength and pulse duration (or combined pulse duration) are modified such that a lesser amount of heat is produced during the electrical pulse (or combined pulses) compared to when a “high energy” or a “medium energy” electrical pulse (or combined pulses) is applied to the sample, provided the buffer composition, the processing assembly, and the sample volume are held constant.

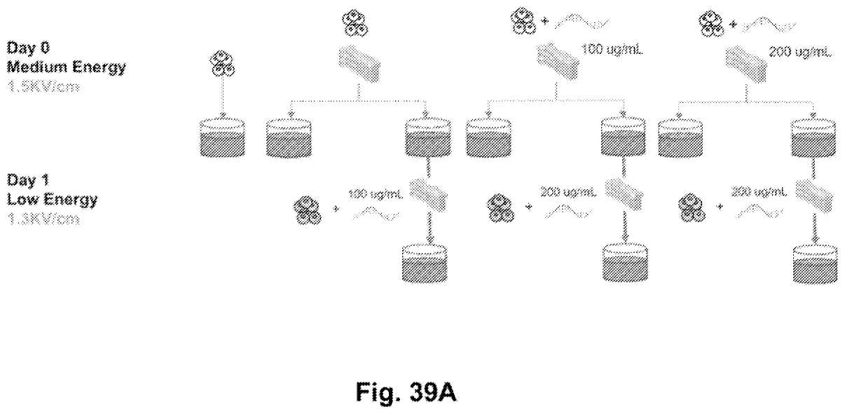

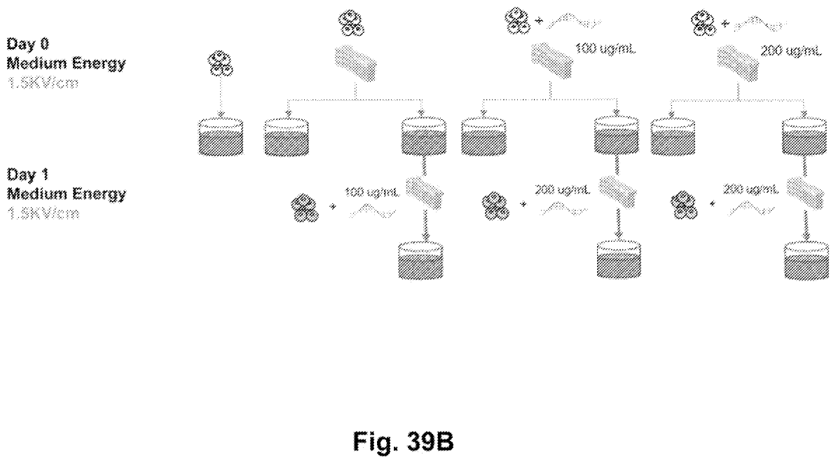

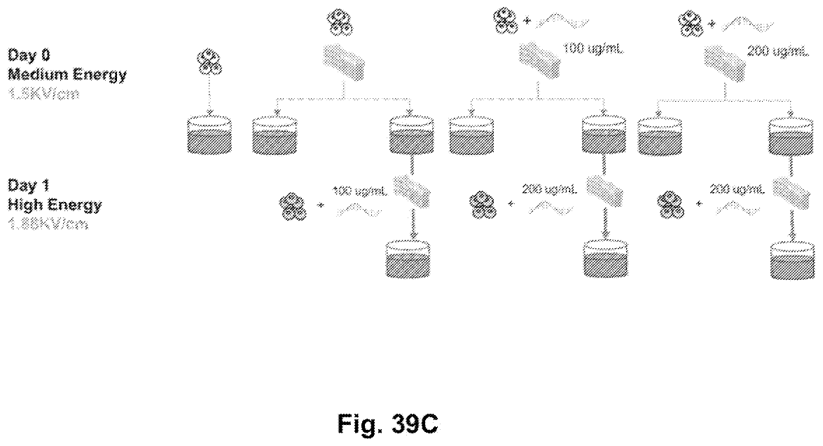

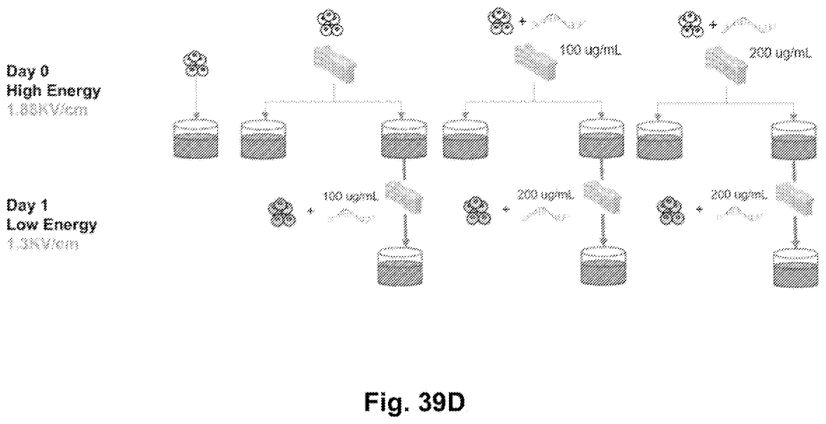

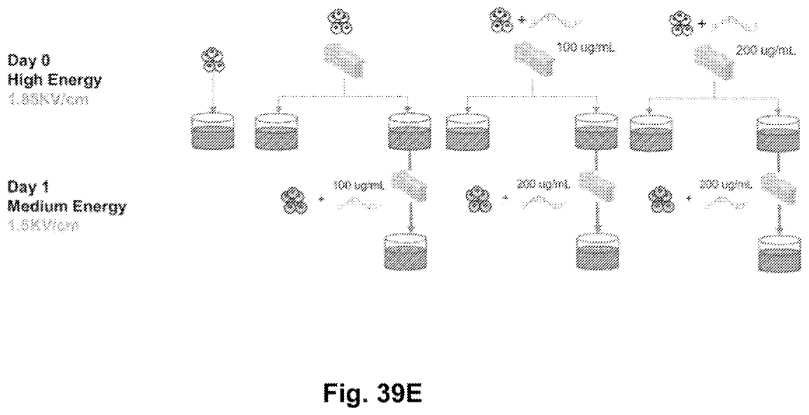

FIGS. 39A-39E illustrate an experimental design for sequential electroporation of expanded lymphocytes at different EP energies with two different GFP mRNA concentrations (100 μg/mL and 200 μg/mL). In FIG. 39A, lymphocytes were subjected to a first, medium energy electrical pulse on Day 0, followed by a second, low energy electrical pulse on Day 1. The first, medium energy electrical pulse on Day 0 used an initial field strength of 1.5 kV/cm, and the second, low energy electrical pulse on Day 1 used an initial field strength of 1.3 kV/cm. In FIG. 39B, lymphocytes were subjected to a first, medium energy electrical pulse on Day 0, followed by a second, high energy electrical pulse on Day 1. The first, medium energy electrical pulse on Day 0 used an initial field strength of 1.5 kV/cm, and the second, high energy electrical pulse on Day 1 used an initial field strength of 1.88 kV/cm. In FIG. 39C, lymphocytes were subjected to a first, medium energy electrical pulse on Day 0, followed by a second, medium energy electrical pulse on Day 1. The first, medium energy electrical pulse on Day 0 used an initial field strength of 1.5 kV/cm, and the second, medium energy electrical pulse on Day 1 used an initial field strength of 1.5 kV/cm. In FIG. 39D, lymphocytes were subjected to a first, high energy electrical pulse on Day 0, followed by a second, low energy electrical pulse on Day 1. The first, high energy electrical pulse on Day 0 used an initial field strength of 1.88 kV/cm, and the second, low energy electrical pulse on Day 1 used an initial field strength of 1.3 kV/cm. In FIG. 39E, lymphocytes were subjected to a first, high energy electrical pulse on Day 0, followed by a second, medium energy electrical pulse on Day 1. The first, high energy electrical pulse on Day 0 used an initial field strength of 1.88 kV/cm, and the second, medium energy electrical pulse on Day 1 used an initial field strength of 1.5 kV/cm.

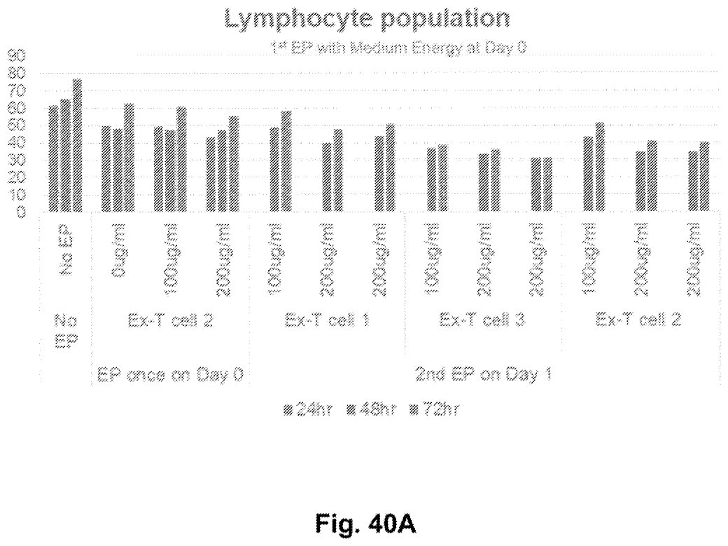

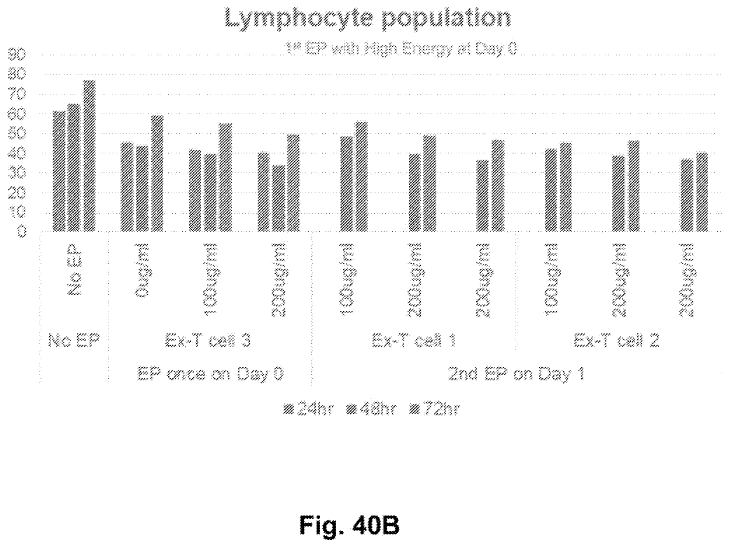

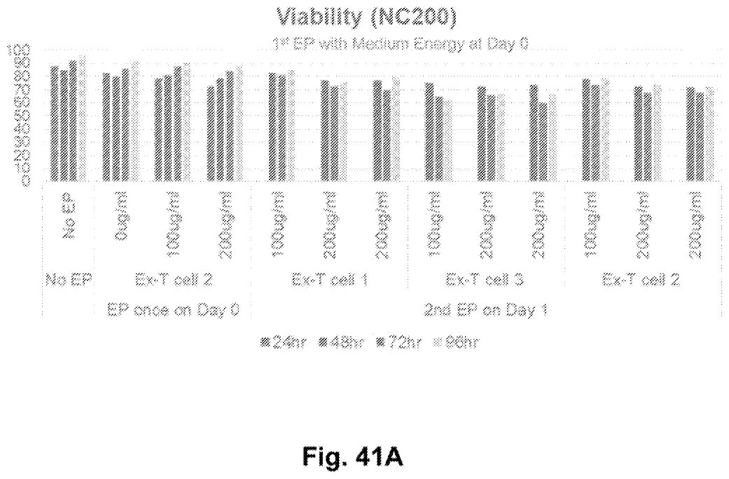

FIGS. 40A-40B show populations of lymphocytes expressing GFP mRNA at three different time points (24 hr, 48 hr, and 72 hr) after sequential electroporation of expanded lymphocytes at different EP energies with two different GFP mRNA concentrations (100 μg/mL and 200 μg/mL).

Lymphocyte populations of cells subjected to a first, medium energy electrical pulse are shown in FIG. 40A (Ex-T cell 2). Lymphocytes were subjected to a first, medium energy electrical pulse, and a second, low energy electrical pulse (FIG. 40A, Ex-T cell 1) as described for and illustrated by FIG. 39A; a first, medium energy electrical pulse, and a second, high energy electrical pulse (FIG. 40A, Ex-T cell 3) as described for and illustrated by FIG. 39B; and a first, medium energy electrical pulse, and a second, medium energy electrical pulse (FIG. 40A, Ex-T cell 2) as described for and illustrated by FIG. 39C.

Lymphocyte populations of cells subjected to a first, high energy electrical pulse are shown in FIG. 40B (Ex-T cell 3). Lymphocytes were subjected to a first, high energy electrical pulse, and a second, low energy electrical pulse (FIG. 40B, Ex-T cell 1) as described for and illustrated by FIG. 39D; and a first, high energy electrical pulse, and a second, medium energy electrical pulse (FIG. 40B, Ex-T cell 2) as described for and illustrated by FIG. 39E.

Comparing the lymphocyte population data after cells were subjected to either a first, medium energy electrical pulse or a first, high energy electrical pulse and a second electrical pulse of low, medium, or high energy, lymphocyte recovery was comparable after sequential electroporation of expanded lymphocytes at different EP energies for all five energy combinations described for and illustrated by FIGS. 39A-39E.

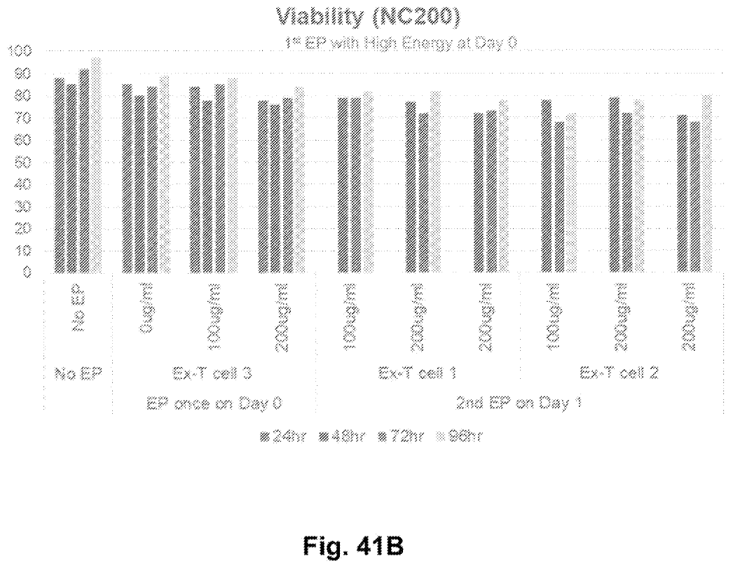

FIGS. 41A-41B show that lymphocyte viability was comparable after sequential electroporation of expanded lymphocytes at different EP energies for all five EP energy combinations described for and illustrated by FIGS. 39A-39E.

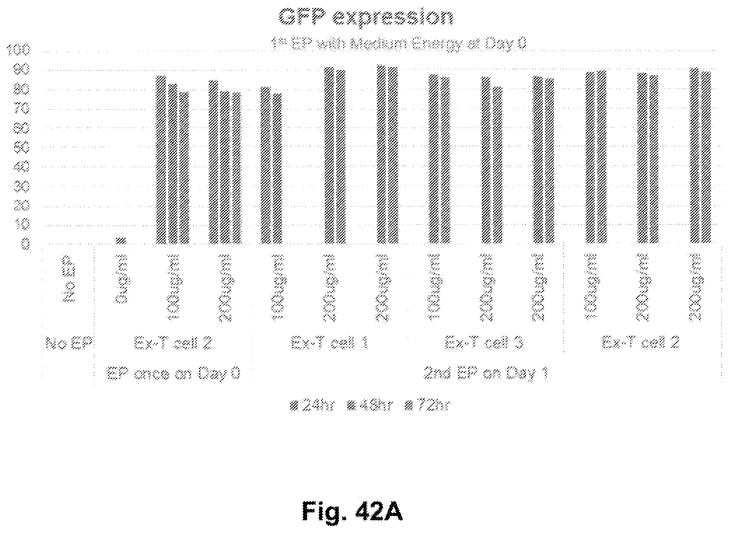

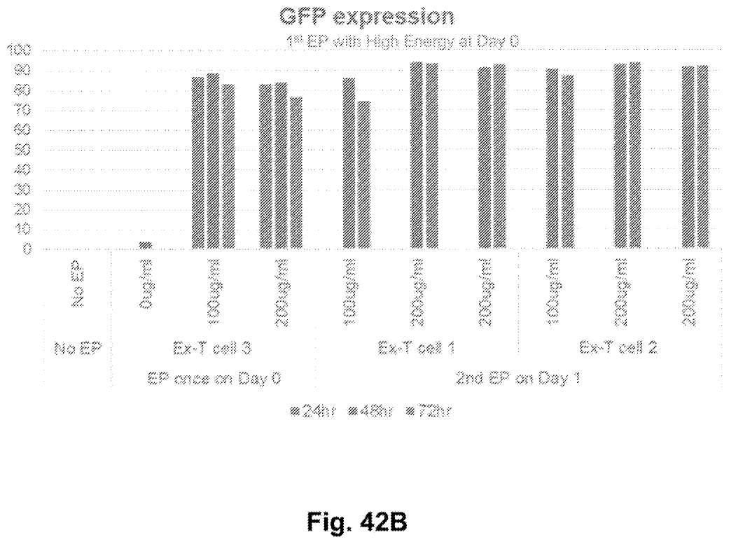

FIGS. 42A-42B show GFP expression by lymphocytes at three different time points (24 hr, 48 hr, and 72 hr) after sequential electroporation of expanded lymphocytes at different EP energies with two different GFP mRNA concentrations (100 μg/mL and 200 μg/mL).

GFP expression by lymphocytes subjected to a first, medium energy electrical pulse are shown in FIG. 42A (Ex-T cell 2). FIG. 42A provides GFP expression by lymphocytes subjected to a first, medium energy electrical pulse, and a second, low energy electrical pulse (Ex-T cell 1) as described for and illustrated by FIG. 39A. FIG. 42A provides GFP expression by lymphocytes subjected to a first, medium energy electrical pulse, and a second, high energy electrical pulse (Ex-T cell 3) as described for and illustrated by FIG. 39B. Finally, FIG. 42A provides GFP expression by lymphocytes subjected to a first, medium energy electrical pulse, and a second, medium energy electrical pulse (Ex-T cell 2) as described for and illustrated by FIG. 39C.

GFP expression by lymphocytes subjected to a first, high energy electrical pulse are shown in FIG. 42B (Ex-T cell 3). FIG. 42B provides GFP expression by lymphocytes subjected to a first, high energy electrical pulse, and a second, low energy electrical pulse (Ex-T cell 1) as described for and illustrated by FIG. 39D. FIG. 42B also provides GFP expression by lymphocytes subjected to a first, high energy electrical pulse, and a second, medium energy electrical pulse (Ex-T cell 2) as described for and illustrated by FIG. 39E.

Comparing GFP expression by lymphocytes after the cells were subjected to either a first, medium energy electrical pulse or a first, high energy electrical pulse and a second electrical pulse of low, medium, or high energy, GFP expression was comparable after sequential electroporation of expanded lymphocytes at different EP energies for all five EP energy combinations described for and illustrated by FIGS. 39A-39E.

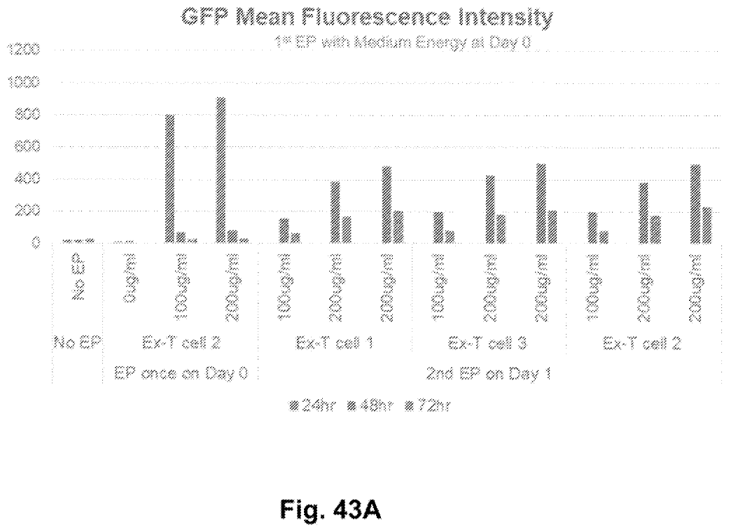

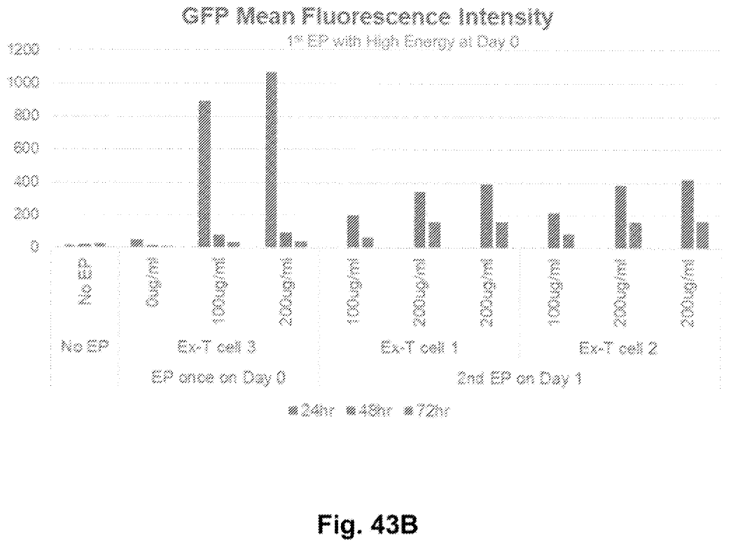

FIGS. 43A-43B show GFP mean fluorescence intensity (MFI) for lymphocytes at three different time points (24 hr, 48 hr, and 72 hr) after sequential electroporation of expanded lymphocytes at different EP energies with two different GFP mRNA concentrations (100 μg/mL and 200 μg/mL).

GFP MFI for lymphocytes subjected to a first, medium energy electrical pulse are shown in FIG. 43A (Ex-T cell 2). FIG. 43A provides GFP MFI by lymphocytes subjected to a first, medium energy electrical pulse, and a second, low energy electrical pulse (Ex-T cell 1) as described for and illustrated by FIG. 39A. FIG. 43A also provides GFP MFI by lymphocytes subjected to a first, medium energy electrical pulse, and a second, high energy electrical pulse (Ex-T cell 3) as described for and illustrated by FIG. 39B. Finally, FIG. 43A provides GFP MFI by lymphocytes subjected to a first, medium energy electrical pulse, and a second, medium energy electrical pulse (Ex-T cell 2) as described for and illustrated by FIG. 39C.

GFP MFI for lymphocytes subjected to a first, high energy electrical pulse are shown in FIG. 43B (Ex-T cell 3). FIG. 43B provides GFP MFI by lymphocytes subjected to a first, high energy electrical pulse, and a second, low energy electrical pulse (Ex-T cell 1) as described for and illustrated by FIG. 39D. FIG. 43B also provides GFP MFI by lymphocytes subjected to a first, high energy electrical pulse, and a second, medium energy electrical pulse (Ex-T cell 2) as described for and illustrated by FIG. 39E. Higher MFI was observed with lymphocytes subjected to a first, high energy electrical pulse.

These data demonstrate that repeated electroporation of cells with mRNA on sequential days leads to significantly higher transgene expression versus a single electroporation. Activated T cells can be sequentially electroporated using the described energy electrical pulse permutations because there are no major differences between lymphocyte population, cell viability, and GFP expression for the various energy electrical pulse permutations.

### Example 3—Repeated Electroporation and Serial Gene Editing of Activated T Cells

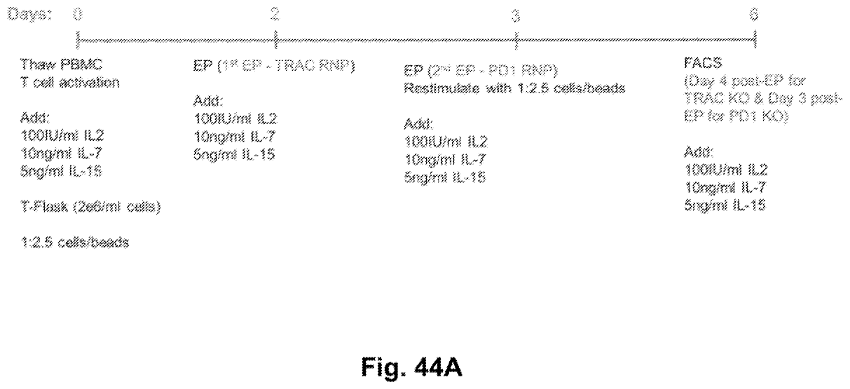

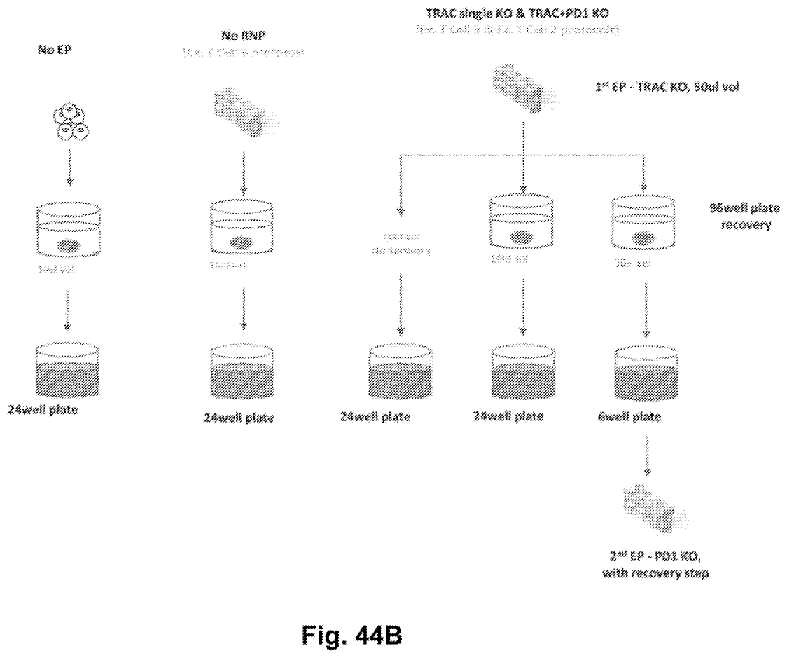

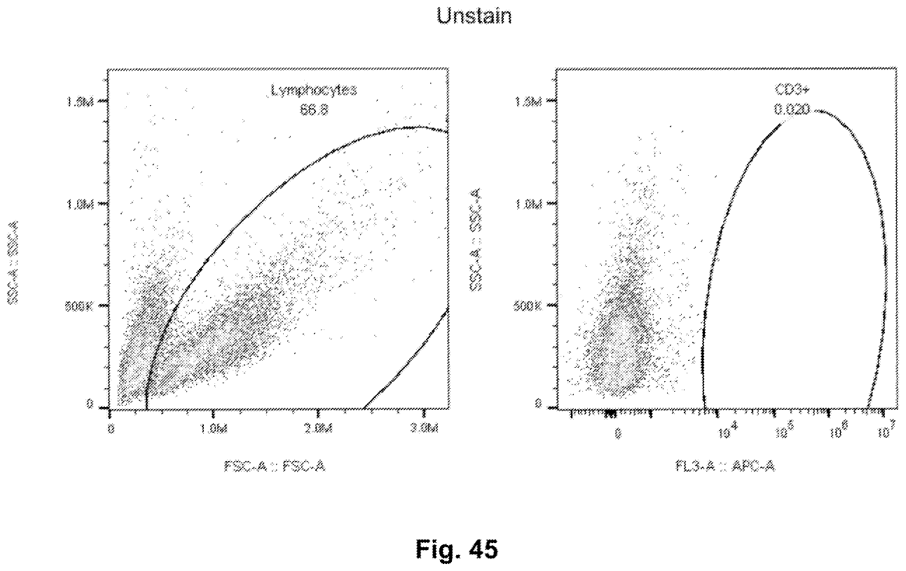

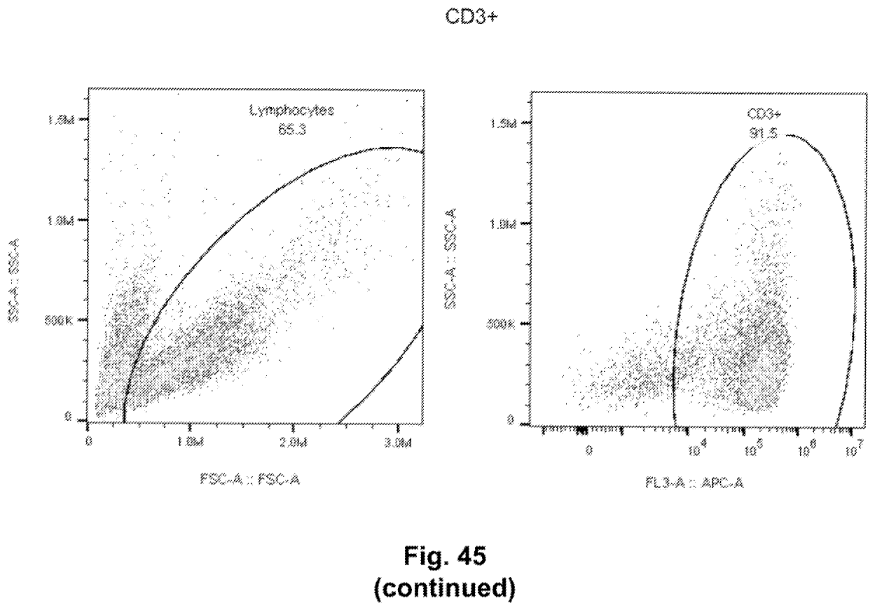

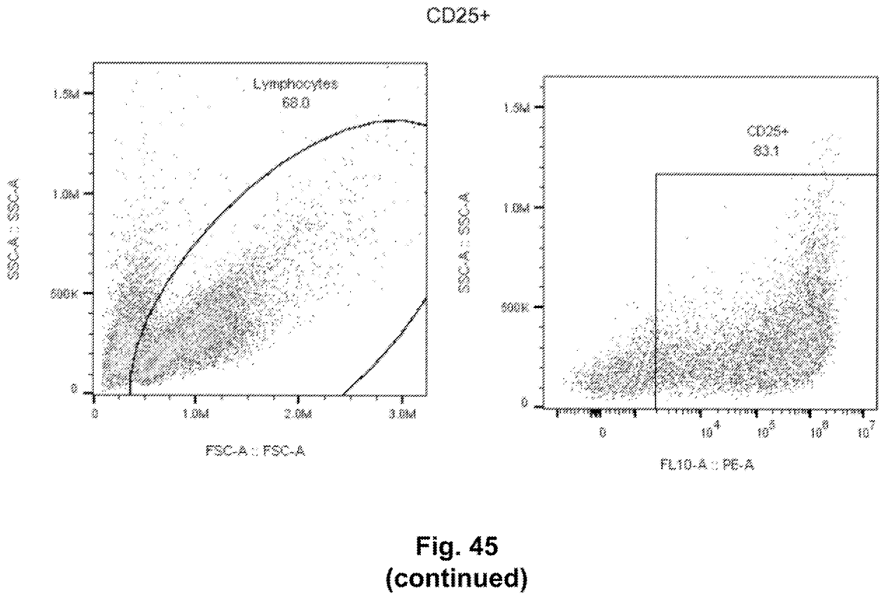

FIG. 44 illustrates an experimental design for sequential electroporation of activated T cells with two different ribonucleoprotein (RNP) constructs to knock out TRAC and PD1. As shown in FIGS. 44A and 44B, PBMC were thawed, and T cells were activated on Day 0 by culturing 2×10⁶/mL cells for two days with 100 IU/mL IL2, 10 ng/mL IL-7, and 5 ng/mL IL-15, as well as anti-CD3/CD28—conjugated magnetic beads at a ratio of 1:2.5 cells/beads. FIG. 45 shows activation of T-cells as measured by Fluorescence-activated cell sorting (FACS) for CD3⁺- and CD25⁺-stained T cells after incubation with cytokines and CD3/CD28 beads for 2 days.

After activation for two days, on Day 2, 1×10⁸ T cells/mL were washed, suspended in electroporation buffer for 50 μL total reaction volume (5×10⁶ cells), and electroporated with 2 μM TRAC ribonucleoprotein (RNP) from a 30.5 μM stock of TRAC RNP comprising 1:2 Cas9:sgRNA prepared by mixing 61 μM wildtype Cas9 (GENSCRIPT®) with 122 μM TRAC sgRNA (SYNTHEGO®). The first, high energy electroporation on Day 2 used an electrical pulse with an initial field strength of 1.7 kV/cm (T cell 3 protocol). Post-electroporation for TRAC knockout, T cells were recovered for 20 minutes at 37° C., 5% CO₂. Then, 2×10⁶ electroporated T cells/mL were cultured for 24 hours, after which 100 IU/mL IL2, 10 ng/mL IL-7, and 5 ng/mL IL-15 was added. Also tested was a no-rest condition in which post-electroporation for TRAC knockout, cells are not rested for 20 minutes at 37° C., 5% CO₂ and are instead immediately transferred for culture for 24 hours.

On Day 3, 4×10⁷ T cells/mL were washed, suspended in electroporation buffer for 50 μL total reaction volume (2×10⁶ cells), and electroporated with 2 μM PD1 ribonucleoprotein (RNP) from a 30.5 μM stock of PD1 RNP comprising 1:2 Cas9:sgRNA prepared by mixing 61 μM wildtype Cas9 (GENSCRIPT®) with 122 μM PD1 sgRNA (SYNTHEGO®). The second, medium energy electroporation on Day 3 used an electrical pulse with an initial field strength of 1.5 kV/cm (T cell 2 protocol). Post-electroporation for PD1 knockout, T cells were recovered for 20 minutes at 37° C., 5% CO₂. Then, 2×10⁶ electroporated T cells/mL were cultured for 24 hours, after which 100 IU/mL IL2, 10 ng/mL IL-7, and 5 ng/mL IL-15 was added, and cells were restimulated with CD3/CD28 beads at a ratio of 1:2.5 cells/beads.

Experimental controls included T cells that were activated but not subjected to electroporation and activated T cells that were subjected to a first, high energy electroporation with no RNP or other agent on Day 2 using an electrical pulse with an initial field strength of 1.7 kV/cm (T cell 3 protocol).

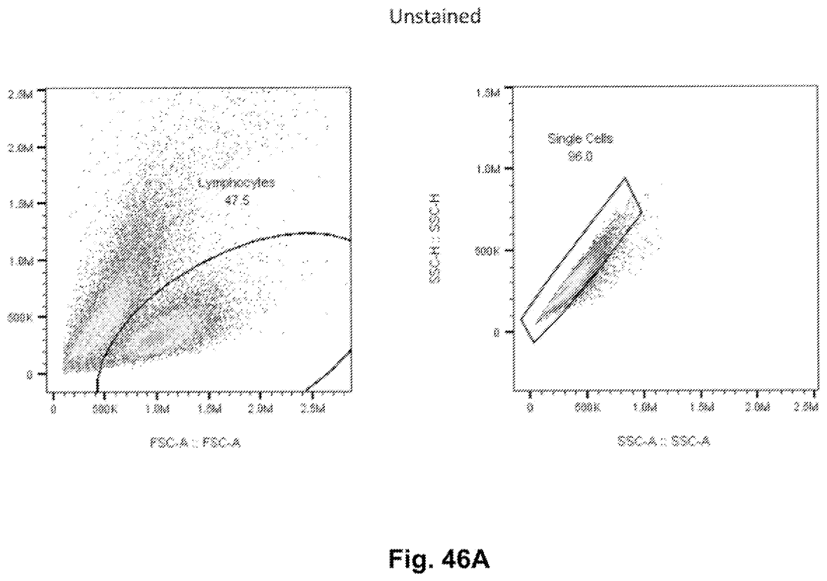

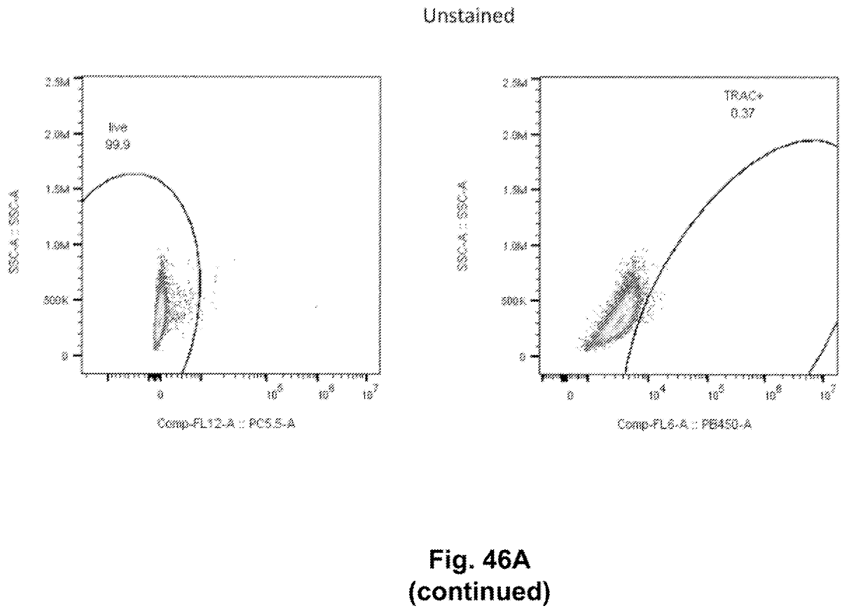

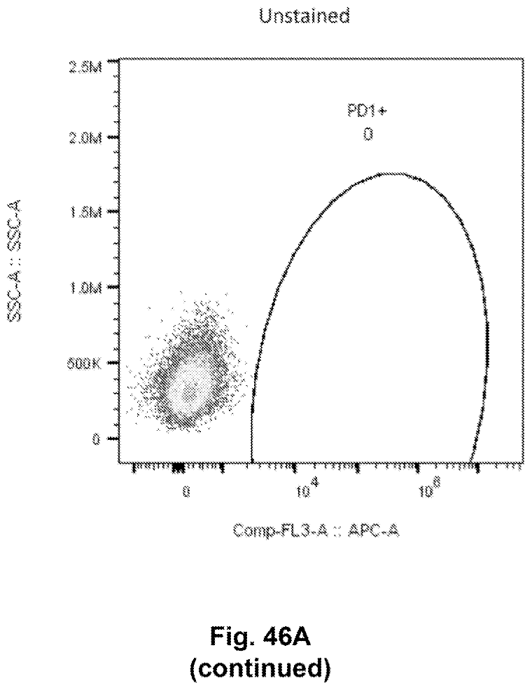

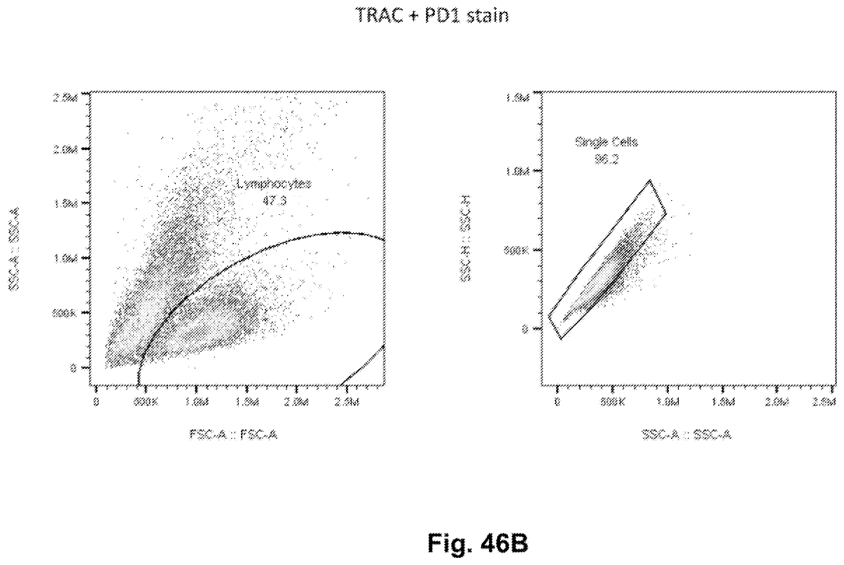

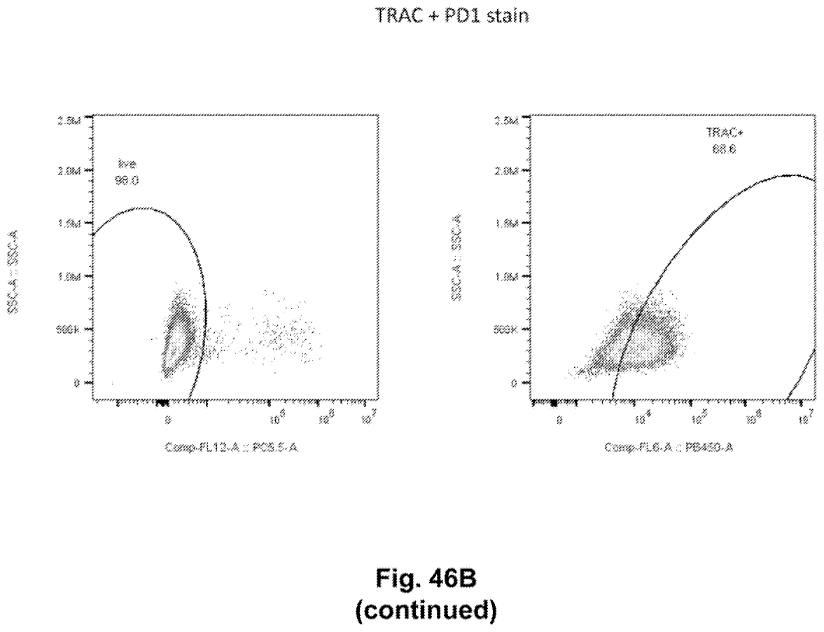

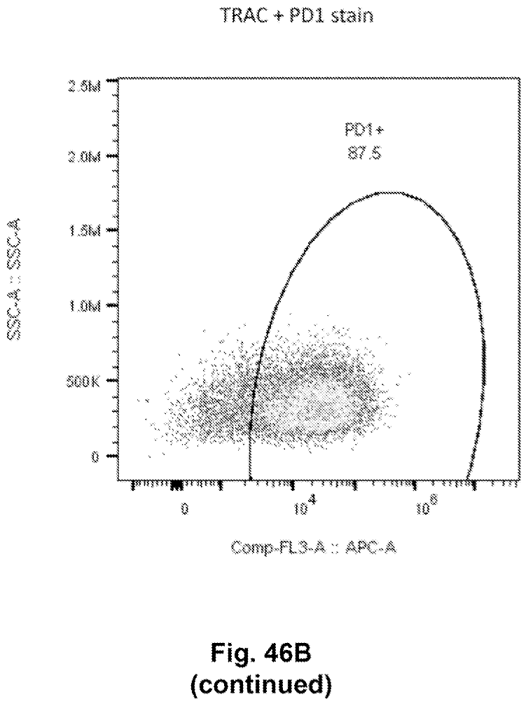

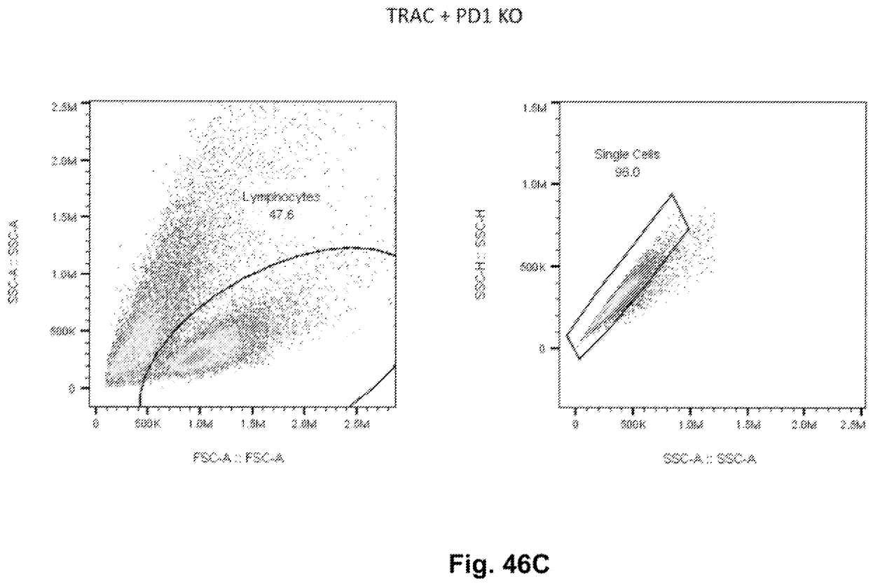

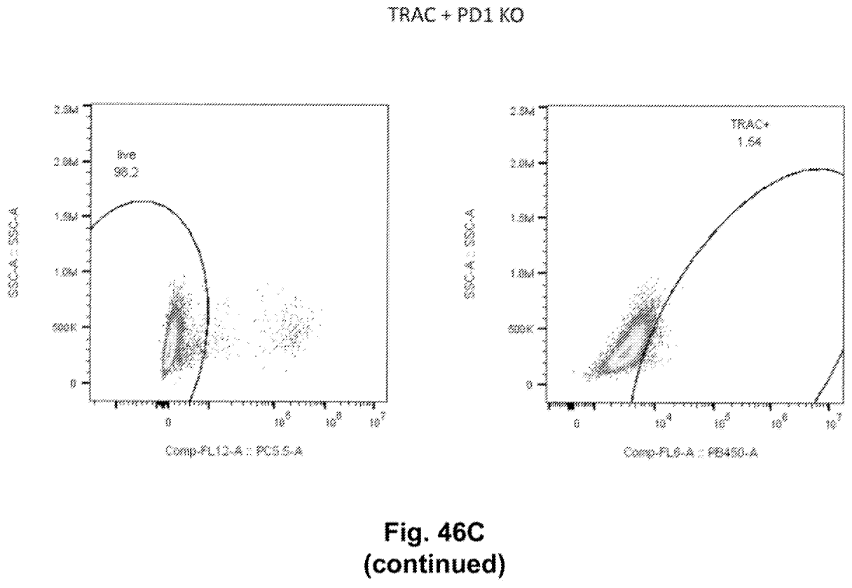

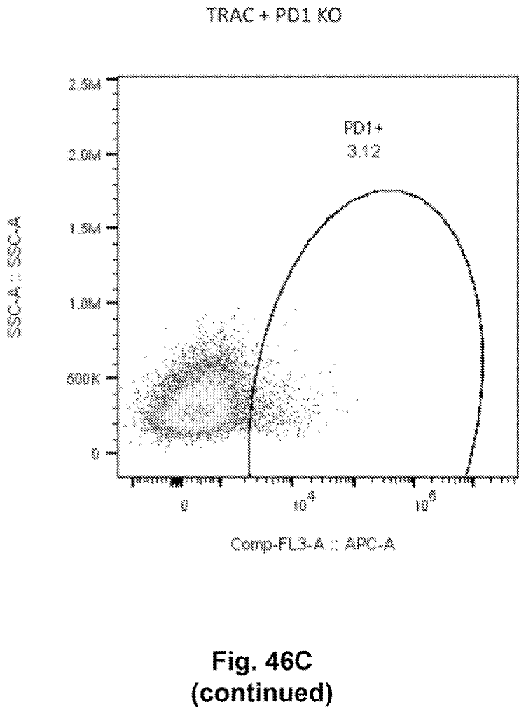

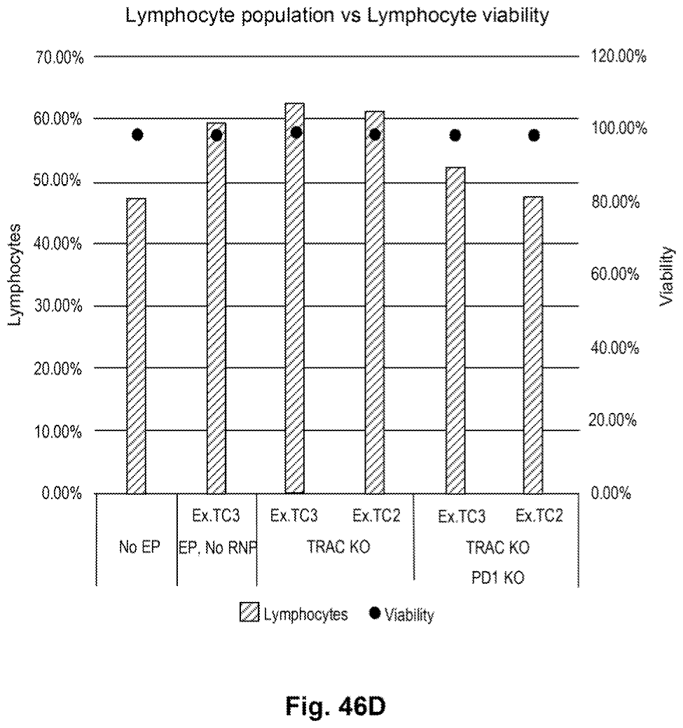

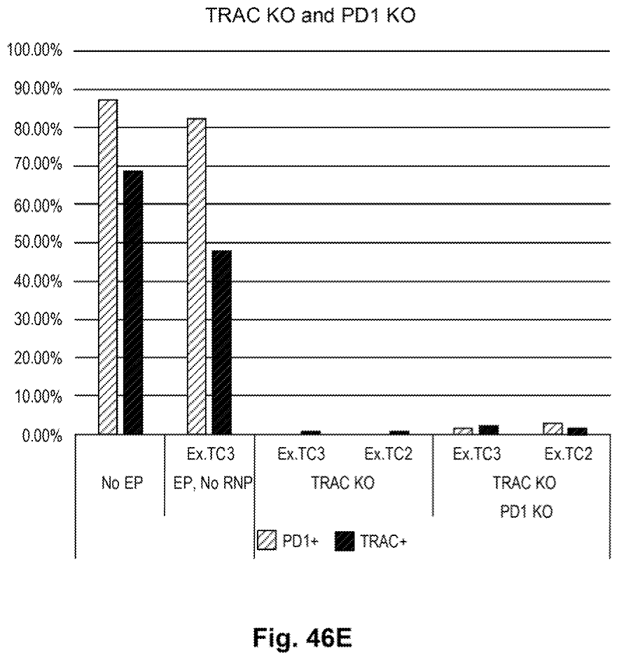

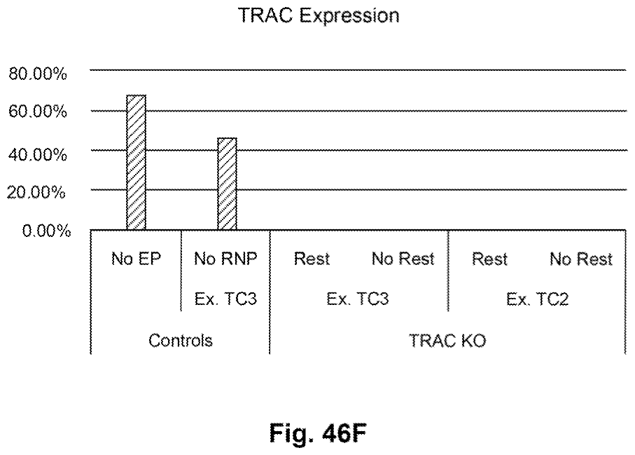

On Day 6, four days post-electroporation for TRAC knockout and three days post-electroporation for PD1 knockout, 100 IU/mL IL2, 10 ng/mL IL-7, and 5 ng/mL IL-15 was added, and FACS was performed to assess TRAC and PD1 knockout efficiency over 30000 collected events. FIG. 46A shows representative gating for unstained T cells. FIG. 46B shows representative gating for TRAC+ and PD1+ T cells for experiments in which T cells were not electroporated with RNP for TRAC and PD1 knockout. FIG. 46C shows representative gating for TRAC+ and PD1+ T cells for experiments in which T cells were electroporated with RNP for TRAC and PD1 knockout. The FACS data obtained on Day 6 for each of the electroporation conditions described in FIG. 44 were quantified to show T cell population versus T cell viability in FIG. 46D and TRAC and PD1 knockout efficiency in FIG. 46E. As shown in FIG. 46D, T cell viability is minimally affected by sequential electroporation to serially edit cells, as cell viability after both the first and second electroporation events was similar to the control in which no electroporation was performed. As shown in FIG. 46E, sequential electroporation of two different RNPs can generate high knockout efficiency for the TRAC and PD1 locus, with both TRAC and PD1 expression decreased to less than 5% after RNP electroporation. Additionally, as shown in FIG. 46F, allowing the TRAC RNP-electroporated T cells to rest for 20 minutes at 37° C., 5% CO₂ after electroporation before culturing for 24 hours did not increase TRAC knockout efficiency compared to TRAC RNP-electroporated T cells that were not rested for 20 minutes at 37° C., 5% CO₂ after electroporation but were instead immediately transferred for culture for 24 hours.

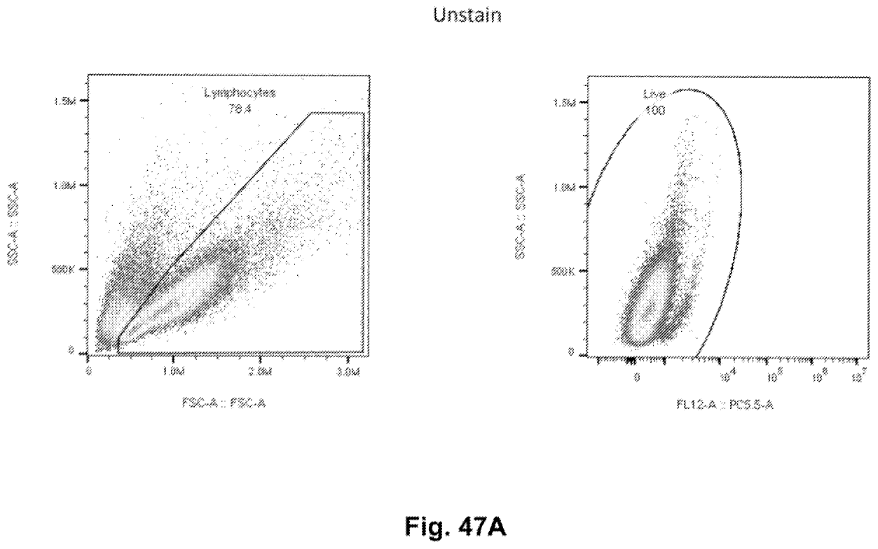

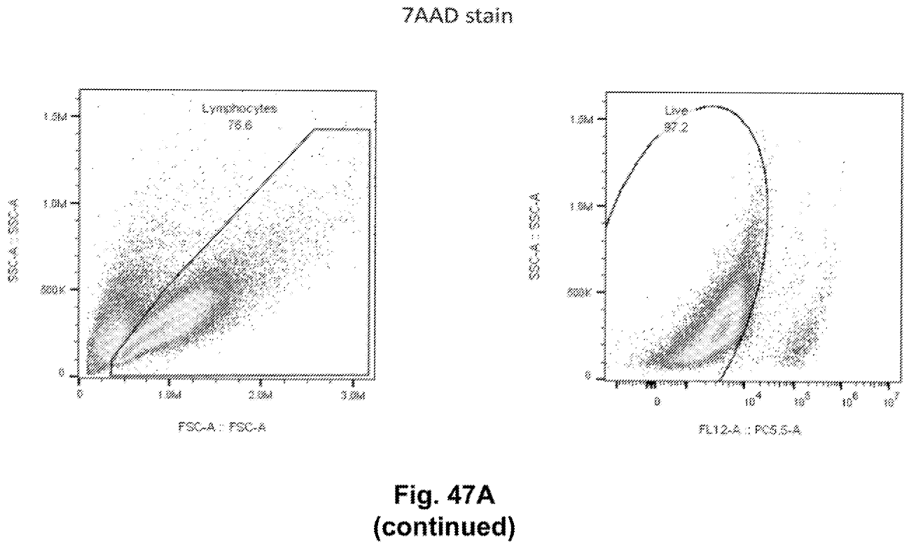

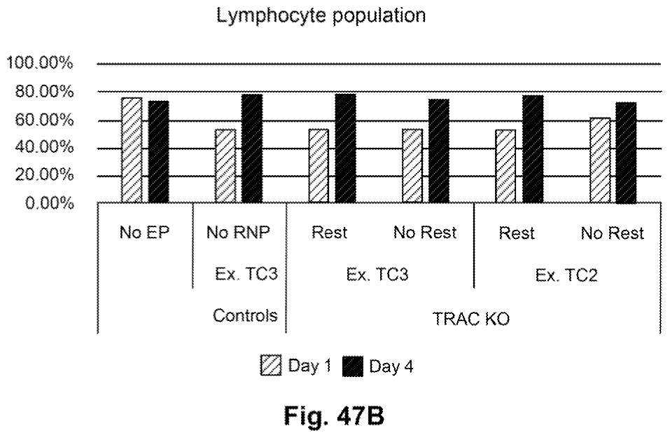

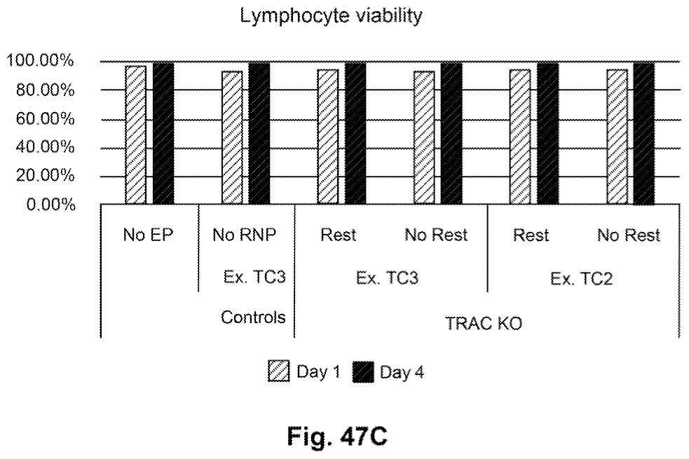

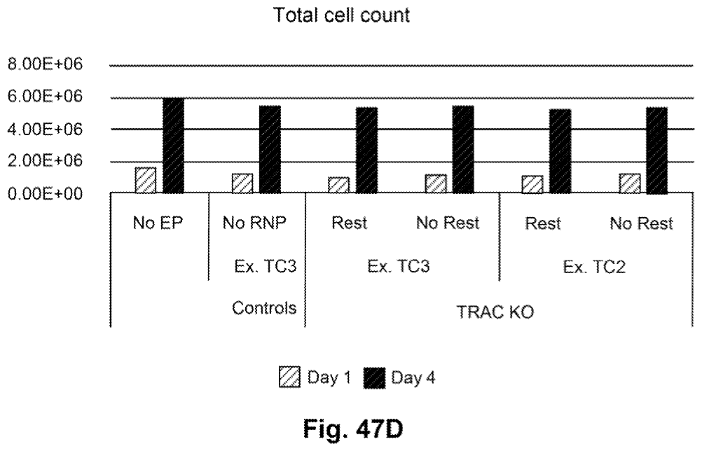

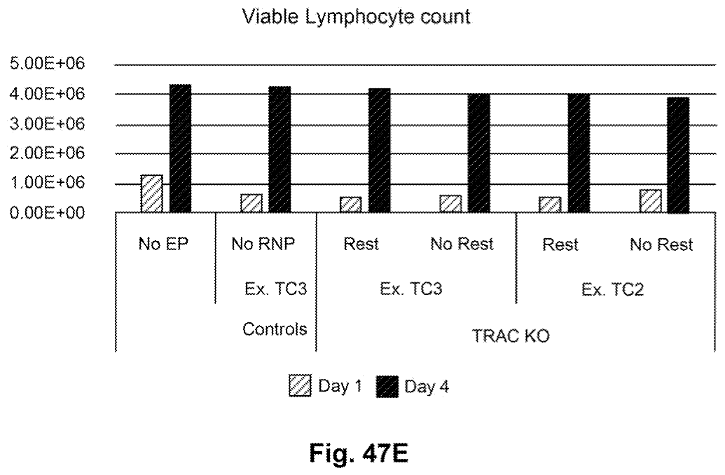

FACS was also performed on Day 6 to measure total cell and lymphocyte counts from 30 μL of cultured cells to assess the effects of a cell rest period for 20 minutes at 37° C., 5% CO₂ on cell viability after electroporation with an RNP construct to knock out TRAC. Gating for unstained and live/dead 7-aminoactinomycin D (7-AAD) stained T cells is shown in FIG. 47A. As shown in FIGS. 47B-47E, the lymphocyte population (FIG. 47B), lymphocyte viability (FIG. 47C), total cell count (FIG. 47D), and total viable lymphocyte count (FIG. 47E) were also minimally affected, where the no rest condition had ˜5% less live lymphocyte count as compared to T cells rested for 20 minutes at 37° C., 5% CO₂ after electroporation.

* * *

The above specification and examples provide a complete description of the structure and use of illustrative aspects. Although certain aspects have been described above with a certain degree of particularity, or with reference to one or more individual aspects, those skilled in the art could make numerous alterations to the disclosed aspects without departing from the scope of this invention. As such, the various illustrative aspects of the methods and systems are not intended to be limited to the particular forms disclosed. Rather, they include all modifications and alternatives falling within the scope of the claims, and aspects other than the one shown may include some or all of the features of the depicted aspect. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one aspect or may relate to several aspects.

The claims are not intended to include, and should not be interpreted to include, means plus- or step-plus-function limitations, unless such a limitation is explicitly recited in a given claim using the phrase(s) “means for” or “step for,” respectively.

What is claimed:

1. An electroporation method comprising:
subjecting a sample comprising one or more intact cells, cell particles, or lipid vesicles to a first electrical pulse having a first field strength and a first pulse duration sufficient to load the cells, cell particles, or lipid vesicles with a first agent according to a first protocol; and
subjecting the sample to a second electrical pulse having a second field strength and a second pulse duration sufficient to load the cells, cell particles, or lipid vesicles with a second agent according to a second protocol;
wherein the first field strength and/or the first pulse duration are different from the second field strength and/or second pulse duration.

2. An electroporation method comprising:
subjecting a sample comprising one or more intact cells, cell particles, or lipid vesicles to a first electrical pulse having a first field strength and a first pulse duration sufficient to load the cells, cell particles, or lipid vesicles with a first agent according to a first protocol;
allowing the sample to recover for at least about 24 hours; and
subjecting the sample to a second electrical pulse having a second field strength and a second pulse duration sufficient to load the cells, cell particles, or lipid vesicles with a second agent according to a second protocol.

3. An electroporation method comprising:
subjecting a sample comprising one or more intact cells, cell particles, or lipid vesicles to a first electrical pulse having a first field strength and a first pulse duration sufficient to load the cells, cell particles, or lipid vesicles with a first agent according to a first protocol;
allowing the sample to recover for at least about 24 hours; and
subjecting the sample to a second electrical pulse having a second field strength and a second pulse duration sufficient to load the cells, cell particles, or lipid vesicles with a second agent according to a second protocol;
wherein the first field strength and/or the first pulse duration are different from the second field strength and/or second pulse duration.

4. A method of serially editing cell genes comprising:
subjecting a sample comprising one or more intact cells to a first electrical pulse having a first field strength and a first pulse duration sufficient to load the cells with a first agent according to a first protocol; and
subjecting the sample to a second electrical pulse having a second field strength and a second pulse duration sufficient to load the cells with a second agent according to a second protocol;
wherein the first field strength and/or the first pulse duration are different from the second field strength and/or second pulse duration.

5. A method of serially editing cell genes comprising:
subjecting a sample comprising one or more intact cells to a first electrical pulse having a first field strength and a first pulse duration sufficient to load the cells with a first agent according to a first protocol;
allowing the sample to recover for at least about 24 hours; and
subjecting the sample to a second electrical pulse having a second field strength and a second pulse duration sufficient to load the cells with a second agent according to a second protocol.

6. A method of serially editing cell genes comprising:
subjecting a sample comprising one or more intact cells to a first electrical pulse having a first field strength and a first pulse duration sufficient to load the cells with a first agent according to a first protocol;
allowing the sample to recover for at least about 24 hours; and
subjecting the sample to a second electrical pulse having a second field strength and a second pulse duration sufficient to load the cells with a second agent according to a second protocol;
wherein the first field strength and/or the first pulse duration are different from the second field strength and/or second pulse duration.

7. The method of claim 1, wherein the first and second agent are the same agent.

8. The method of claim 1, wherein the first and second agent are different agents.

9. The method of claim 1, wherein the first and second agents are a nucleic acid, polypeptide, protein, or small molecule.

10. The method of claim 9, wherein the nucleic acid is RNA, and wherein the RNA is mRNA, miRNA, shRNA, siRNA, or an antisense oligonucleotide, or the nucleic acid is DNA, and wherein the DNA is an antisense oligonucleotide, a vector, or a double sense linear DNA.

11. The method of claim 9, wherein the protein is a ribonucleoprotein and comprises a Cas9 protein and a guide RNA.

12. An electroporation method comprising:
(a) subjecting a cell sample comprising one or more intact cells to a first electrical pulse having a first field strength and a first pulse duration sufficient to load cells with a first agent comprising RNA according to a first protocol;
(b) allowing the cell sample to recover for at least about 24 hours; and
(c) subjecting the cell sample to a second electrical pulse having a second field strength and a second pulse duration sufficient to load cells with a second agent comprising RNA according to a second protocol;
wherein the first field strength and/or the first pulse duration are different from the second field strength and/or second pulse duration.

13. An electroporation method comprising:
(a) subjecting a cell sample comprising one or more intact cells to a first electrical pulse having a first field strength and a first pulse duration sufficient to load cells with a first agent comprising DNA according to a first protocol;
(b) allowing the cell sample to recover for at least about 24 hours; and
(c) subjecting the cell sample to a second electrical pulse having a second field strength and a second pulse duration sufficient to load cells with a second agent comprising DNA according to a second protocol;
wherein the first field strength and/or the first pulse duration are different from the second field strength and/or second pulse duration.

14. An electroporation method comprising:
(a) subjecting a cell sample comprising one or more intact cells to a first electrical pulse having a first field strength and a first pulse duration sufficient to load cells with a first agent comprising one or more proteins according to a first protocol;
(b) allowing the cell sample to recover for at least about 24 hours; and
(c) subjecting the cell sample to a second electrical pulse having a second field strength and a second pulse duration sufficient to load cells with a second agent comprising one or more proteins according to a second protocol;
wherein the first field strength and/or the first pulse duration are different from the second field strength and/or second pulse duration.

15. A method of serially editing cells comprising:
(a) subjecting a cell sample comprising one or more intact cells to a first electrical pulse having a first field strength and a first pulse duration sufficient to load cells with a first agent comprising a ribonucleoprotein according to a first protocol;
(b) allowing the cell sample to recover for at least about 24 hours; and
(c) subjecting the cell sample to a second electrical pulse having a second field strength and a second pulse duration sufficient to load cells with a second agent comprising a ribonucleoprotein according to a second protocol;
wherein the first field strength and/or the first pulse duration are different from the second field strength and/or second pulse duration.

16. The method of claim 12, wherein the first and second agent are the same agent.

17. The method of claim 12, wherein the first and second agent are different agents.

18. An electroporation system having a non-transitory computer readable medium comprising instructions that, when executed by a processor, cause the processor to:
select a first protocol associated with a first electrical pulse having a first field strength and a first pulse duration;
subject a sample comprising one or more intact cells, cell particles, or lipid vesicles to the first electrical pulse defined by the first protocol sufficient to load the cells, cell particles, or lipid vesicles with a first agent according to the first protocol;
select a second protocol associated with a second electrical pulse having a second field strength and a second pulse duration; and
subject the sample to the second electrical pulse defined by the second protocol sufficient to load the cells, cell particles, or lipid vesicles with a second agent according to the second protocol;
wherein the first field strength and/or the first pulse duration are different from the second field strength and/or second pulse duration.

19. The electroporation system of claim 18, wherein the first field strength equals the second field strength, and wherein the first pulse duration is longer than the second pulse duration.

20. The electroporation system of claim 18, wherein the first field strength equals the second field strength, and wherein the first pulse duration is shorter than the second pulse duration.
